(19) United States
(12) Patent Application Publication
(10) Pub. No.: US 2017/0298060 A1
(43) Pub. Date: Oct. 19, 2017

(54) PYRROLOPYRIDINE RETINOIC ACID RECEPTOR-RELATED ORPHAN RECEPTOR MODULATORS AND USES THEREOF

(71) Applicant: INNOV17 LLC, Brooklyn, NY (US)

(72) Inventor: Jefferson Tilley, Bend, OR (US)

(21) Appl. No.: 15/490,003

(22) Filed: Apr. 18, 2017

Related U.S. Application Data
(60) Provisional application No. 62/323,962, filed on Apr. 18, 2016.

(51) Int. Cl. C07D 471/04; C07D 519/00

(52) U.S. Cl. CPC C07D 471/04; C07D 519/00

(57) ABSTRACT

Provided herein are compounds of the formula (I):

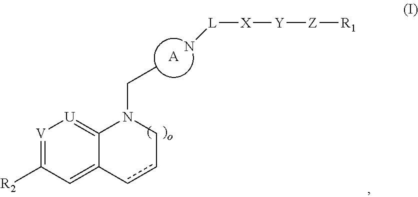

as well as pharmaceutically acceptable salts thereof, wherein the substituents are as those disclosed in the specification. These compounds, and the pharmaceutical compositions containing them, are useful for the treatment of Retinoic Acid Receptor-Related Orphan Receptor regulated diseases and disorders.

### FIELD OF THE INVENTION

The invention relates to Retinoic Acid Receptor-Related Orphan Receptor (ROR) regulated diseases and disorders. More particularly, the invention relates to ROR modulators; compositions comprising a therapeutically effective amount of a ROR modulator; and methods for treating or preventing ROR regulated diseases and disorders. All documents cited to or relied upon below are expressly incorporated herein by reference in their entirety.

### BACKGROUND OF THE INVENTION

There are high unmet medical needs in the few established therapies for several autoimmune, inflammatory, metabolic and oncologic diseases. Despite the diverse clinical manifestations of these diseases, Retinoic Acid Receptor-Related Orphan Receptors (RORs) regulate and contribute to the pathogenesis of these diseases through modulation of immune responses and lipid/glucose homeostasis. The critical regulatory role of RORs has been well-characterized and as therapeutic drug targets been validated in several animal and clinical models of some of these diseases. RORs are transcription factors which belong to the nuclear hormone receptor superfamily (Jetten (2009) Nucl. Recept. Signal., 7:e003; Jetten et al. (2013) Front Endocrinol. (Lausanne), 4:1; Jetten & Joo (2006) Adv. Dev. Biol., 16:313-355). The ROR subfamily consists of three major isoforms: RORα (NR1F1), RORβ (NR1F2), and RORγ (NR1F3), encoded by the RORA, RORB and RORC genes, respectively. RORs are multidomain proteins that contain four principal domains typical of nuclear receptors: a highly variable N-terminal A/B domain, a highly conserved DNA-binding domain (DBD), a ligand binding domain (LBD) that contains the ligand-dependent activation function-2 (AF-2), and a hinge domain between the DBD and LBD. Each ROR gene through alternative splicing and promoter usage generates several ROR isoforms that differ only in their amino-terminus. In humans, there are four RORα isoforms (RORα1-4), one RORβ31 isoform, and two RORγ isoforms (RORγ1 and RORγ2 [RORγt]) that are expressed in a highly tissue-specific manner. RORα and RORγ play an important role in the regulation of lipid/glucose homeostasis, cellular metabolism, immune function and circadian rhythms, and have been implicated in the pathogenesis of several autoimmune, inflammatory, metabolic and oncologic diseases (Burris et al. (2012) Chem. Biol., 19:51-59; Burris et al. (2013) Pharmacol. Rev., 65:710-778; Chang et al. (2016) ACS Chem. Biol., DOI: 10.1021/acschembio.5b00899; Huh & Littman (2012) Eur. J. Immunol., 42:2232-2237; Jetten (2009) Nucl. Recept. Signal., 7:e003; Jetten et al. (2013) Front Endocrinol. (Lausanne), 4:1). Synthetic ligands have been described that interact with the RORα and RORγ LBD functioning as a switch that induces a ROR LBD conformational change. Such change promotes the recruitment and displacement of regulatory coactivator and corepressor proteins and upon ROR DBD binding to the ROR responsive element of the target genes lead to the induction or inhibition of ROR-regulated gene transcriptional activity. Therefore, small molecule drugs that bind to the nuclear receptor LBDs such as ROR could elicit a variety of pharmacological responses, including activation (agonists), inactivation (antagonists or non-agonists), and for receptors that are constitutively active, ligands can downregulate the constitutive response (inverse agonists).

RORγt is the master regulator of human T Helper 17 (T_{H}17) cell differentiation, function and cytokine production (Ivanov et al. (2006) Cell, 126:1121-1133). The critical role of T_{H}17 cells in the pathogenesis of autoimmune and inflammatory diseases has been established and is conferred by the production of its signature proinflammatory cytokines IL-17A, IL-17F, IL-17AF, IL-21, IL-22 (Ghoreschi et al. (2010) Nature, 467:967-971; Lee et al. (2012) Nat. Immunol., 13:991-999; Miossec et al. (2009) N. Engl. J. Med., 361:888-898; Miossec & Kolls (2012) Nat. Rev. Drug Discov., 11:763-776; Zepp et al. (2011) Trends Immunol., 32:232-239). Although several transcription factors regulate T_{H}17, γ/δ T and innate lymphoid cells as important sources of T_{H}17 cytokines, these cells are distinguished by its specific regulation of RORγt for cytokine transcriptional output and effector functions and to a lesser extent by RORα in humans (Cua & Tato (2010) Nat. Rev. Immunol., 10:479-489; Huh & Littman (2012) Eur. J. Immunol., 42:2232-2237; Ivanov et al. (2006) Cell, 126:1121-1133; Spits & Di Santo (2011) Nat. Immunol., 12:21-27; Sutton et al. (2012) Eur. J. Immunol., 42:2221-2231). Also, in several autoimmune disease models, there is a relative imbalance of increased T_{H}17 cells over low numbers of immunosuppressive CD4⁺CD25⁺Foxp3⁺ regulatory T cells [T_{Reg}] (Edwards et al. (2011) J. Neurol., 258:1518-1527; Littman & Rudensky (2010) Cell, 140:845-858). Inhibiting RORγt could have a broader anti-inflammatory effect on the combined inhibition of all T_{H}17 cytokine production and inflammatory cellular function, and in the induction and expansion of suppressive T_{Reg} cells, important in autoimmune and inflammatory disease resolution, and may also have therapeutic potential in metabolic diseases such as diet-induced insulin resistance known to be regulated by RORγ. Since both RORγ1 and RORγt [RORγ1] protein isoforms, contain identical LBDs, small molecule RORγ modulators that inhibit RORγt activity will also inhibit RORγ. Inversely, T_{H}17 cells play a beneficial role in anti-tumor immunity by recruiting natural killer cells and tumor-killing cytotoxic CD8+ T cells into the tumor microenvironment. Compounds enhancing RORγt activity and thereby promoting T_{H}17 cells and T_{H}17 cytokine production could be of therapeutic benefit in several cancer models (Chang et al. (2016) ACS Chem. Biol., DOI: 10.1021/acschembio.5b00899). RORα similarly plays an important regulatory role in the pathogenesis of autoimmune and inflammatory disorders, and also in metabolic and oncologic diseases. RORα critically regulates lipid and glucose homeostasis and cellular metabolism that contribute to the development of metabolic diseases. Whereas, RORα is downregulated in several types of cancer, RORα expression is associated with tumor suppression (Cook et al. (2015) Nucl Receptor Res, DOI:10.11131/2015/101185). As ligand-dependent transcription factors, it is desirable to prepare compounds that modulate RORα and/or RORγ activity which can be used in the treatment of RORα- and/or RORγ-regulated autoimmune, inflammatory, metabolic and oncologic diseases.

### SUMMARY OF THE INVENTION

The present invention is directed to compounds of the formula (I):

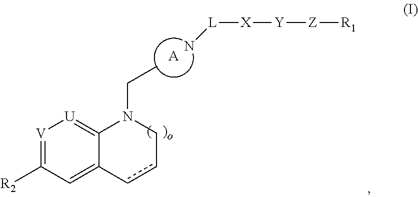

wherein:
A is a mono- or bicyclic 5- to 8-membered heterocyclic ring having one ring carbon replaced by N as shown, said ring optionally mono- or bi-substituted on one or more ring carbons independently with a C₁-C₆ alkyl group, or halogen;

### L is —C(O)— or —S(O)₂;

X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
One of U and V is N and the other is CH;
R₁ is —C₁-C₆ alkyl, optionally mono- or bi-substituted with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
   R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O); [symbol]
   is a single or double bond;
   o is 0 or 1;
   n is 0 or 1;
   p is 0, 1 or 2; and
   q is 0 or 1,
   or a pharmaceutically acceptable salt thereof.

The present invention is also directed to pharmaceutically acceptable salts of the compounds of formula (I), pharmaceutical compositions and to methods of treating diseases and disorders. The compounds and compositions disclosed herein are ROR modulators and useful for the treatment of ROR-mediated diseases and disorders.

### DETAILED DESCRIPTION

The invention is based in part on the discovery of ROR modulators, which interact with RORα and/or RORγ and thereby inhibit or induce RORα and/or RORγ activity, and RORα- and/or RORγ-regulated target gene and protein expression. The invention is also based on compositions comprising an effective amount of a ROR modulator; and methods for treating or preventing disorders regulated by RORα and/or RORγ, comprising the administration of a therapeutically effective amount of a ROR modulator.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

The following definitions are used in connection with the ROR modulators:

“ROR” refers to RORα and/or RORγ isoforms.

“RORα” refers to all isoforms encoded by the RORA gene.

“RORγ” refers to all isoforms encoded by the RORC gene which include RORγ1 and RORγt [RORγ2].

“RORα modulator” refers to a chemical compound that modulates, either directly or indirectly, the activity of ROR. RORα modulators include antagonists/non-agonists, inverse agonists and agonists of RORα.

“RORγ modulator” refers to a chemical compound that modulates, either directly or indirectly, the activity of RORγ. RORγ modulators include antagonists/non-agonists, inverse agonists and agonists of RORγ.

The term “ROR modulator” includes any and all possible isomers, stereoisomers, enantiomers, diastereomers, tautomers, pharmaceutically acceptable salts, hydrates, solvates, and prodrugs of the ROR modulators described herein.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

Unless otherwise specifically defined, the term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. The substituents can themselves be optionally substituted.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl.

“C₁-C₄ alkyl” refers to a straight or branched chain saturated hydrocarbon containing 1-4 carbon atoms. Examples of a C₁-C₄ alkyl group include, but are not limited to, methyl, ethyl, propyl, butyl, isopropyl, isobutyl, sec-butyl and tert-butyl.

“C₁-C₅ alkyl” refers to a straight or branched chain saturated hydrocarbon containing 1-5 carbon atoms. Examples of a C₁-C₅ alkyl group include, but are not limited to, methyl, ethyl, propyl, butyl, pentyl, isopropyl, isobutyl, sec-butyl and tert-butyl, isopentyl and neopentyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl.

The term “cycloalkyl” refers to a cyclic hydrocarbon containing 3-6 carbon atoms. Examples of a cycloalkyl group include, but are not limited to, cyclopropyl, cyclobutyl, cyclopentyl and cyclohexyl.

The term “heterocycle” as used herein refers to a cyclic hydrocarbon containing 3-12 atoms wherein at least one of the atoms is an O, N, or S wherein a monocyclic heterocycle may contain up to two double bonds. Examples of heterocycles include, but are not limited to, aziridine, oxirane, thiirane, azetidine, oxetane, morpholine, thiomorpholine, thietane, pyrrolidine, tetrahydrofuran, tetrahydrothiophene, piperidine, tetrahydropyran, thiane, imidazolidine, oxazolidine, thiazolidine, dioxolane, dithiolane, piperazine, oxazine, dithiane, and dioxane.

The term “heteroaryl” as used herein refers to an aromatic mono- or polycyclic radical of 5 to 12 atoms having at least one aromatic ring containing one, two, or three ring heteroatoms selected from N, O, and S, with the remaining ring atoms being C. Examples of heteroaryls include, but are not limited to, furan, thiophene, pyrrole, pyrroline, oxazole, thiazole, imidazole, pyrazole, isoxazole, isothiazole, triazole, thiadiazole, pyrane, pyridine, pyridazine, pyrimidine, pyrazine and triazene.

It is understood that any of the substitutable hydrogens on a cycloalkyl, heterocycle and heteroaryl can be substituted independently with one or more substituents, for example 1, 2 or 3 substituents. Examples of substituents include, but are not limited to, halogen, C₁-C₃ alkyl, hydroxyl, alkoxy, oxo and cyano groups.

A “patient” is a mammal, e.g., a human, mouse, rat, guinea pig, dog, cat, horse, cow, pig, or non-human primate, such as a monkey, chimpanzee, baboon or rhesus monkey, and the terms “patient” and “subject” are used interchangeably herein.

The invention also includes pharmaceutical compositions comprising a therapeutically effective amount of a ROR modulator and a pharmaceutically acceptable carrier. The invention includes a ROR modulator provided as a pharmaceutically acceptable prodrug, hydrate, salt, such as a pharmaceutically acceptable salt, enantiomers, stereoisomers, or mixtures thereof.

Representative “pharmaceutically acceptable salts” include, e.g., water-soluble and water-insoluble salts, such as the acetate, amsonate (4,4-diaminostilbene-2, 2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

A “therapeutically effective amount” when used in connection with a ROR modulator is an amount effective for treating or preventing a ROR-regulated disease or disorder.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The term “treating”, with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a ROR modulator.

The term “optionally substituted,” as used in this disclosure, means a suitable substituent can replace a hydrogen bound to a carbon, nitrogen, or oxygen. When a substituent is oxo (i.e., ═O) then 2 hydrogens on the atom are replaced by a single O. Suitable substituents are selected from the following which include, but are not limited to, hydroxyl, halogen, perfluorinated C₁-C₆ alkyl, amine, —C₁-C₁₂ alkyl, —C₂-C₁₂ alkene, —C₂-C₁₂ alkyne, —(C₁-C₃ alkyl)-(cycloalkyl), aryl, alkyl-aryl, —C(O)H, —C(O)OH, —C(O)alkyl, —C(O)—O-alkyl, —C(O)NH(alkyl), benzyl, —C(O)NH₂, —C(O)N(alkyl)₂, —NHC(O)H, —NHC(O)alkyl, —SO₂(alkyl), —SO₂NH₂, —SO₂NH(alkyl), —SO₂N(alkyl)₂, S, CN, and SCN. It will be understood by those skilled in the art, with respect to any group containing one or more substituents, that such groups are not intended to introduce any substitution or substitution patterns that are sterically impractical, synthetically non-feasible and/or inherently unstable. Furthermore, combinations of substituents and/or variables within any of the Formulae represented herein are permissible only if such combinations result in stable compounds or useful synthetic intermediates wherein stable implies a reasonable pharmacologically relevant half-life at physiological conditions.

The following abbreviations are used herein and have the indicated definitions: ACTB is β-actin, AF-2 is activation function-2, AIBN is azobisisobutyronitrile, Boc and BOC are tert-butoxycarbonyl, Boc₂O is di-tert-butyl dicarbonate, BOP is (benzotriazol-1-yloxy)tris(dimethylamino)phosphonium hexafluorophosphate, BSA is bovine serum albumin, CD is cluster of differentiation, CDI is 1,1′-carbonyldiimidazole, DBD is DNA-binding domain, DCC is N,N′-dicyclohexylcarbodiimide, DIEA and DIPEA is N,N-diisopropylethylamine, DMAP is 4-dimethylaminopyridine, DMEM is Dulbecco's Modified Eagle Medium, DMF is N,N-dimethylformamide, DMSO is dimethyl sulfoxide, DOSS is sodium dioctyl sulfosuccinate, EC₅₀ is half maximal effective concentration, EDC and EDCI are 1-ethyl-3-(3-dimethylaminopropyl)carbodiimide hydrochloride, ELISA is enzyme-linked immunosorbent assay, EtOAc is ethyl acetate, FBS is fetal bovine serum, FOXP3 is forkhead box P3, G-CSF is granulocyte colony-stimulating factor, h is hour, HATU is 2-(7-aza-1H-benzotriazole-1-yl)-1,1,3,3-tetramethyluronium hexafluorophosphate, HIV is human immunodeficiency virus, HOBt is 1-hydroxybenzotriazole, HPMC is hydroxypropyl methylcellulose, HPRT1 is hypoxanthine phosphoribosyltransferase 1, IC₅₀ is half maximal inhibitory concentration, IFN-γ is interferon gamma, IL is interleukin, IL-23R is interleukin 23 receptor, LAH is lithium aluminum hydride, LBD is ligand binding domain, MIQE is minimum information for publication of quantitative real-time PCR experiments, MTBE is methyl tert-butyl ether, NBS is N-bromosuccinnide, NMP is N-methyl-2-pyrrolidone, oxone is potassium peroxymonosulfate, PBMCs is peripheral blood mononuclear cells, PCR is polymerase chain reaction, Pd/C is palladium on carbon, PGK1 is phosphoglycerate kinase, PPIA is peptidylprolyl isomerase A, REST is Relative Expression Software Tool, RORα is retinoic acid receptor-related orphan receptor alpha, RORγ is retinoic acid receptor-related orphan receptor gamma, TBAB is tetrabutylammonium bromide, TBP is terminal binding protein, TFA is trifluoroacetic acid, TFRC is transferrin receptor, TGF-β1 is transforming growth factor beta 1, T_{H}17 is T helper 17 cell, TGPS is tocopherol propylene glycol succinate, THF is tetrohydrofuran, TLC is thin layer chromatography, TR-FRET is time-resolved fluorescence resonance energy transfer and M is micromolar.

In another embodiment, provided is a method of treating a Retinoic Acid Receptor-Related Orphan Receptor mediated disease or disorder, comprising the step of administering a therapeutically effective amount of a compound according to formula (I) to a patient in need thereof, wherein said disease or disorder is rheumatoid arthritis, psoriasis, psoriatic arthritis, polymyalgia rheumatica, multiple sclerosis, lupus, uveitis, inflammatory bowel disease, ankylosing spondylitis, vasculitis, atherosclerosis, macular degeneration, diabetes, obesity, cancer, asthma or chronic obstructive pulmonary disease.

In another aspect, methods of inhibiting, preventing or treating a disease, or symptoms of a disease, regulated by RORα and/or RORγ, is provided, which comprises administering to a subject in need thereof, a therapeutically-effective amount of a ROR modulator. In some embodiments, the disease regulated by RORα and/or RORγ is selected from Autoimmune, Inflammatory, Metabolic and Oncologic Diseases, including but not limited to angina pectoris, myocardial infarction, atherosclerosis, cystic fibrosis, gastritis, autoimmune myositis, giant cell arteritis, Wegener's granulomatosis, asthma, chronic obstructive pulmonary disease, rheumatoid arthritis, juvenile rheumatoid arthritis, allergen-induced lung inflammation, allergy, psoriasis, psoriatic arthritis, colitis, inflammatory bowel disease, Crohn's disease, ulcerative colitis, Sjogren's syndrome, dry eye, optic neuritis, neuromyelitis optica, myasthenia gravis, Guillain-Barre syndrome, Graves disease, multiple sclerosis, autoimmune uveitis, ankylosing spondylitis, organ transplant rejection, polymyalgia rheumatic, systemic lupus erythematosus, cutaneous lupus, lupus nephritis, glomerulonephritis, diabetes mellitus type 1, pulmonary inflammation, macular degeneration, obesity, non-alcoholic fatty liver disease, steatohepatitis, insulin resistance, diabetes mellitus type 2, glucose intolerance, and metabolic syndrome; and Oncologic Diseases, including but not limited to multiple myeloma, bone disease associated with multiple myeloma, gastric cancer and colon cancer.

Also described are methods of modulating RORα and/or RORγ activity as an agonist, inverse agonist or antagonist/non-agonist in a subject, which comprises administering to a subject in need thereof a pharmaceutically effective amount of a ROR modulator.

Also described are methods of inducing or inhibiting RORα- and/or RORγ-regulated target gene expression and protein production in a subject which comprises administering to a subject in need thereof a pharmaceutically effective amount of a ROR modulator.

Also described are methods of regulating corepressor and/or coactivator protein interaction with RORα and/or RORγ LBD in a subject that comprises administering to a subject in need thereof a pharmaceutically effective amount of a ROR modulator.

Also described are methods of reducing the amount of RORα- and/or RORγ-regulated production of T_{H}17 cytokines IL-17A, IL-17F, IL-17AF, IL-21, and/or IL-22 in a subject which comprises administering to a subject in need thereof a pharmaceutically effective amount of a ROR modulator.

Also described are methods of inducing or inhibiting, either directly or indirectly, RORα- and/or RORγ-regulated cell proliferation or activation in a subject which comprises administering to a subject in need thereof a pharmaceutically effective amount of a ROR modulator.

The ROR modulators can each be administered in amounts that are sufficient to treat or prevent but are not limited to Autoimmune, Inflammatory, Metabolic and Oncologic Diseases, or prevent the development thereof in subjects.

The invention also includes pharmaceutical compositions useful for treating or preventing a ROR regulated disease, or for inhibiting a ROR regulated disease, or more than one of these activities. The compositions can be suitable for internal use and comprise an effective amount of a ROR modulator and a pharmaceutically acceptable carrier. The ROR modulators are especially useful in that they demonstrate very low systemic toxicity or no systemic toxicity.

Administration of the ROR modulators can be accomplished via any mode of administration for therapeutic agents. These modes include systemic or local administration such as oral, nasal, parenteral (intravenous), intramuscular, intrathecal, intra-vitreal, transdermal, subcutaneous, vaginal, buccal, rectal, topical administration modes or as a drug-eluting stent.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, intrathecal, intra-vitreal injection, subcutaneous or intramuscular form, all using forms well known to those skilled in the pharmaceutical arts.

Illustrative pharmaceutical compositions are tablets and gelatin capsules comprising a ROR modulator and a pharmaceutically acceptable carrier, such as: a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the ROR modulator is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the ROR modulators.

The ROR modulators can be also formulated as a suppository that can be prepared from fatty emulsions or suspensions; using polyalkylene glycols such as propylene glycol, as the carrier.

In further embodiments, the pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, delayed release formulations, extended release formulations, pulsatile release formulations, multiparticulate formulations, and mixed immediate and controlled release formulations

The ROR modulators can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564, the contents of which are herein incorporated by reference in their entirety.

ROR modulators can also be delivered by the use of monoclonal antibodies as individual carriers to which the ROR modulators are coupled. The ROR modulators can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the ROR modulators can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, ROR modulators are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Compositions can be prepared according to conventional mixing, granulating or coating methods, respectively, and the present pharmaceutical compositions can contain from about 0.1% to about 80%, from about 5% to about 60%, or from about 1% to about 20% of the ROR modulator by weight or volume.

The dosage regimen utilizing the ROR modulator is selected in accordance with a variety of factors including type, species, age, weight, sex, race, diet, and medical condition of the patient; the severity of the condition to be treated; the route of administration; the renal or hepatic function of the patient; and the particular ROR modulator employed. A physician or veterinarian of ordinary skill in the art can readily determine and prescribe the effective amount of the drug required to prevent, counter or arrest the progress of the condition.

Effective dosage amounts of the present invention, when used for the indicated effects, range from about 0.1 mg to about 5000 mg of the active ingredient per unit dose which could be administered. In one embodiment, the compositions are in the form of a tablet that can be scored. Appropriate dosages of the ROR modulators can be determined as set forth in Goodman, L. S.; Gilman, A. The Pharmacological Basis of Therapeutics, 5th ed.; MacMillan: New York, 1975, pp. 201-226, the contents of which are hereby incorporated by reference.

ROR modulators can be administered in a single daily dose, or the total daily dosage can be administered in divided doses of two, three or four times daily. Furthermore, ROR modulators can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of the ROR modulator ranges from about 0.1% to about 15%, w/w or w/v.

The ROR modulators can also each be administered in amounts that are sufficient to treat or prevent ROR-associated diseases. These diseases include, but are not limited to, Autoimmune, Inflammatory, Metabolic and Oncologic diseases, either individually or in combination with one or more agents and or methods for treating and preventing these ROR-regulated diseases.

### GENERAL SCHEMES

### Methods for Making the RORα, RORγ and RORα/RORγ Modulators

Compounds of the present invention can be prepared beginning with commercially available starting materials and utilizing general synthetic techniques and procedures known to those skilled in the art. Chemicals may be purchased from companies such as for example SigmaAldrich, VWR and Lancaster. Chromatography supplies and equipment may be purchased from such companies as for example AnaLogix, Inc, Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; and Waters Corporation, Milford, Mass. Biotage, ISCO and Analogix columns are pre-packed silica gel columns used in standard chromatography.

Examples of synthetic pathways useful for making ROR modulators of the present invention are set forth in the Examples below and generalized in Schemes 1-4 below.

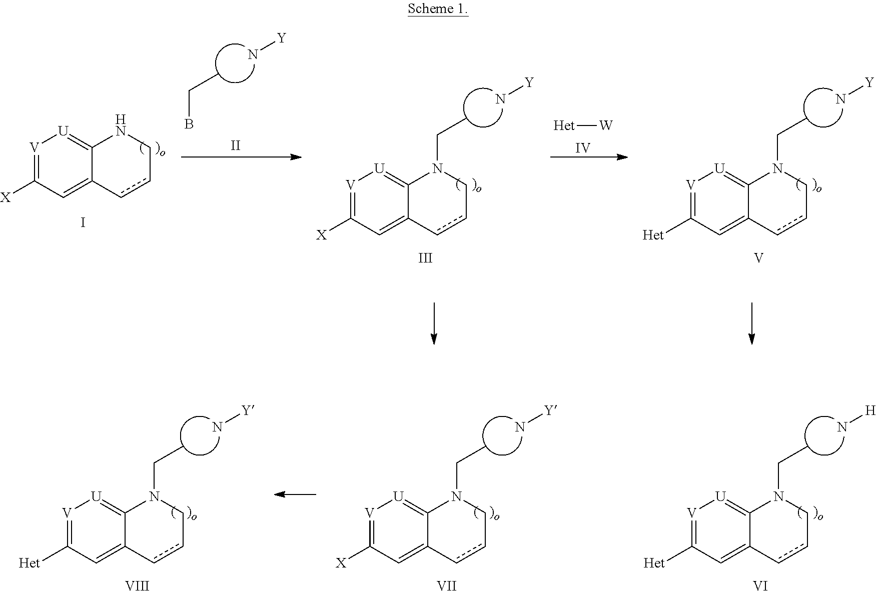

The starting materials I shown in scheme 1 wherein one of U and V is N and the other is CH, o is 1 or 0, [symbol] is a single bond when o is 1 or single or a double bond when o is 0, and X is a group capable of participating in a transition metal catalyzed cross coupling reaction, such as a chloride, bromide, iodide or triflate, are commercially available or readily prepared from commercially available compounds. For example, 5-bromo-pyrrolo[2,3-c]pyridine is available using the method described in WO 2012103806, 5-bromo-pyrrolo[2,3-b]pyridine and 6-Bromo-1,2,3,4-tetrahydro-1,7-naphthyridine are commercially available and 6-bromo-1,2,3,4-tetrahydro-1,8-naphthyridine can be prepared using the method described in WO2001027103. Alkylation of I on nitrogen with a compound II in which B is a leaving group such as a bromide, chloride or tosylate and Y is either a protecting group, an acyl or sulfonyl group of the invention or an acyl or sulfonyl group which can be transformed into an acyl group or sulfonyl group, of the invention to give a compound III can be accomplished by standard methods, such as treatment of a solution of compound I in suitable inert solvent such as DMF with base such as NaH followed by compound II. The reaction may be carried out at room temperature, or at a mildly elevated temperature.

The resulting III may then be coupled to the heteroaromatic derivatives Het-W (IV) in which Het is an optionally substituted 5-7-membered heteroaromatic compound, which may incorporate a protecting group as appropriate, and W is a functional group such as a boronic acid or a halogen atom, capable of participating in a transition metal catalyzed cross-coupling reaction such as a Suzuki reaction. Skilled organic chemists will understand how to select the particular choice of X, W and transition metal catalyst for a given desired transformation and incorporate the appropriate protection/deprotection methods, where needed. In some cases, it may be desirable to convert X to a metal derivative prior to coupling. For example, see Stadlwieser, J. F., et al, Helvetica Chimica ACTA 2006, 89, 936-946. This is typically done using a bisborane such as bis(pinacolato)diboron in the presence of a suitable catalyst such as PdCl₂(dppf) DCM to give a boronic acid derivative prior to the coupling reaction with Het-W. See for example: N. Kudo et al., Angew. Chem. Int. Ed., 2006, 45, 1282-1284 and Dvorak, C. A.; et al., Journal of Organic Chemistry 2005, 70, 4188-4190; Barder, T. E., et al. J Am. Chem. Soc. 2005, 127, 4685-4696, Isley, N. W. et al, Journal of the American Chemical Society, 2013, 135, 17707-17710. In some cases, other metalling reagents leading for example to organostannane or organozinc intermediates may be preferable for a particular desired coupling reaction. For a recent review on the implementation of organo zinc mediated coupling reactions, see Sidduri, A., et al., Synthesis 2014, 46, 430-444.

Carrying out the coupling reaction will then lead to the target compounds V, which depending on the selection of Y, may be compounds of the invention or intermediates that can be converted to compounds of the invention. For example, in cases where Y is an acyl or sulfonyl group of the invention or a projected variant of such, removal of any protecting groups will lead directly to compounds of the invention. In cases where Y is a protecting group, for example a benzyl, carboxybenzyl or Boc group, removal using the appropriate conditions, well known to medicinal chemists, would lead to VI, which can be transformed to a compound of the invention via acylation or sulfonylation, followed by any needed functional group or protecting group manipulation.

In some cases, it may be desirable to manipulate the group Y in structure III to give VII in which Y′ is an acyl or sulfonyl group of the invention or may be simply transformed into an acyl group of the invention by routine transformations, prior to coupling the heterocyclic ring give VIII.

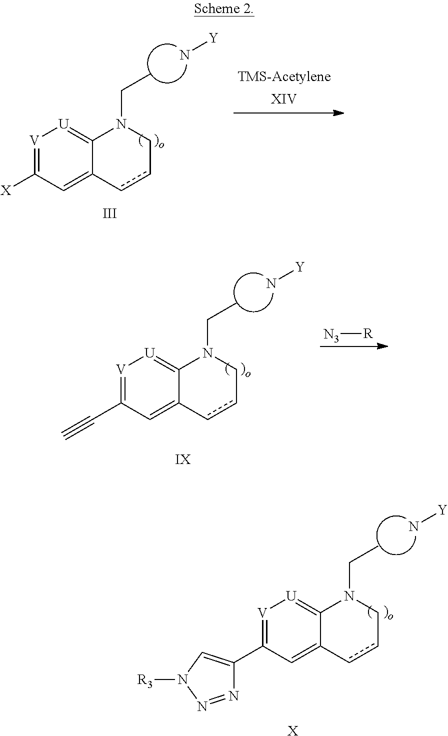

Alternatively, heterocycles Het in the above structures may be constructed directly attached to the pyrrolo[2,3-b]pyridine, pyrrolo[2,3-c]pyridine, 1,2,3,4-tetrahydro-1,7-naphthyridine or 1,2,3,4-tetrahydro-1,8-naphthyridine rings. Such transformations are well known in heterocyclic chemistry and skilled medicinal chemists will understand how to vary the order of the steps to suit the particular choice of target structure. For example, as shown in Scheme 2, 1,2,3-triazoles may be ready constructed by first converting a compound of structure III to an acetylene for example by treatment with TMS-acetylene in the presence of a suitable transition metal catalyst. Typically the TMS group is lost during workup and when it is still present, it can be removed under standard condition to give a compound of structure IX. Treatment of IX with a substituted azide derivative in the presence of a suitable catalyst, for example, a copper catalyst then gives the corresponding trazole of formula X which is either a compound of the invention or readily converted to a compound of the invention following suitable functional group transformations. Triazole formation using this method is widely used in organic chemistry and is typically referred to as “click chemistry”. One variant is described in, Tornøe, C. W., et al, J. Org Chem, 2002, 67, 3057-3064. The application of click chemistry to the synthesis of certain electron deficient triazoles is described in Chattopadhysy, B., Organic Letters 2010, 12, 2166-2169. Depending on the choice of R₃, further functionalization of this substituent can be carried out after triazole formation using standard methods.

Alternative sequences are also envisioned, in which X of III is a nitrile or can be converted to a nitrile. Subsequent reactions leading to 1,2,4-triazoles, oxadiazoles and tetrazoles can be carried followed established literature precedent.

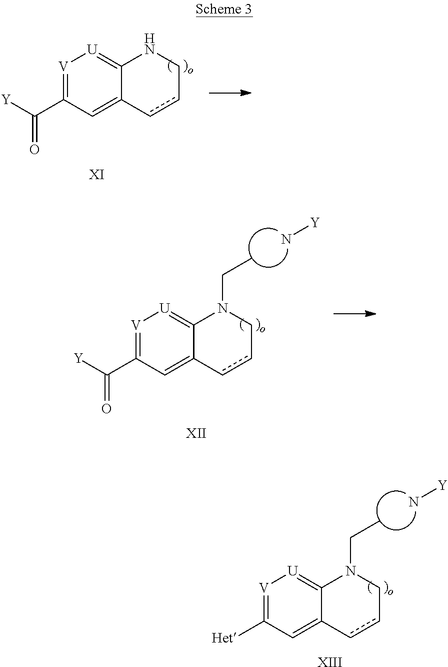

In some cases, it may be desirable to construct the heteroaromatic species from a carbonyl derivative such as XI, Y═H, OH, NHR₄, or OR₅, wherein R₄ is H, lower alkyl or OR₆, wherein R₆ is H or lower alkyl and R₅ is lower alkyl or another substituent suitable for the displacement chemistry associated with the intended heterocycle construction. Such intermediates can be alkylated as above with the appropriate reagents of structure II to give compounds of structure XII as described in Scheme 1, followed by elaboration of the carbonyl derivative to the desired heterocyclic derivatives, XIII using the chemistry appropriate to the target heterocycle. In general, the sequence of steps necessary to carry out these transformations is well established in the chemistry literature. The sequence of the steps may be altered to suit the particular selection of target, available starting materials and experimental convenience. 1,2,4-Oxadiazoles and 1,2,4-triazoles are among the types of heterocycles available through this chemistry. The order of the steps may be varied to suit the particular target and efficiency of the various steps involved.

The intermediate compounds II, are either commercially available or can be prepared in a few steps using standard techniques well known to practicing medicinal chemists. Where desired, one or more halogen atoms, particularly fluorine atoms, may be substituted for hydrogen in II from the corresponding hydroxy- or oxo-derivatives using standard fluorination reagents such as DAST or deoxo-Fluor. These reagents may be employed to convert carbonyl or gem di-thio groups or hydroxyl groups to gem-difluoro moieties. For a review of the use of these reagents, see Singh, et al., Synthesis 2002, 17, 2561. There are many other approaches to the introduction of fluorine atoms into organic hetereocycles which may be more suitable for particular cases. Some of these are summarized in Liang, et al., Angewandte Chemie International Ed. 2013, 52, 8214-8264. For a particularly relevant example, See WO2014197345. Where employed, the choice of protecting group will depend on the remaining steps anticipated during the rest of the synthesis of the particular target compound. Typically, benzyl-, carboxybenzyl- or Boc groups are used. A particularly useful guide to to selection of nitrogen protecting groups is Greene's Protective Groups in Organic Synthesis by Peter G. M. Wuts and T. W. Greene, 4^{th} ed., Wiley, 2007.

Compounds II bearing alkyl groups are also available through purchase or a series of simple synthetic steps. For example, Boc-protected 2-methyl-4-hydroxymethyl piperidine is commercially available, for example from Affinity Research Chemicals of Richmond, Del. or via synthesis using the method described in WO03103669. 4-Hydroxymethyl-2,2,6,6-tetramethylpiperidine can be prepared using the method described in WO2012068589 and 2,6-dimethyl-4-hydroxymethylpiperidine can be prepared as described in U.S. Pat. No. 7,985,755. These various intermediates can be protected and functionalized through a series of routine steps for use in the procedures outlined in the above schemes. 5-Methyl- and 5,5-dimthylpyrrolidine derivatives can be prepared from the corresponding pyrroldinones as shown in Scheme 4 and the examples reported herein.

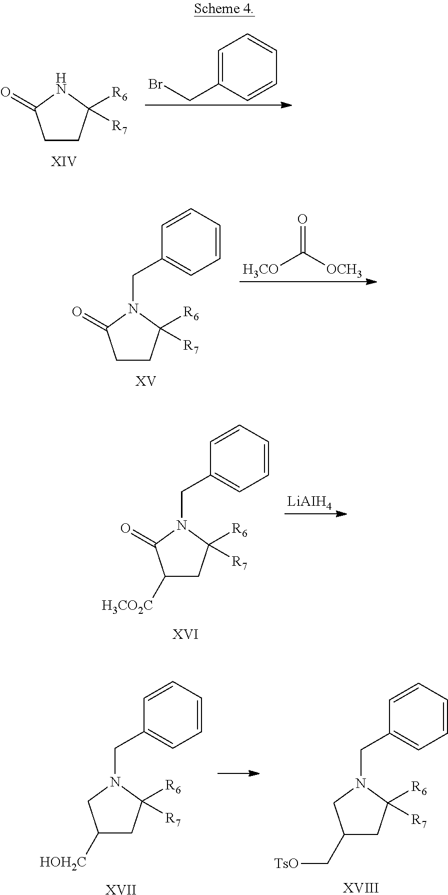

Thus, shown in Scheme 4, a compound of structure XIV in which one of R₆ is lower alkyl and R₇ is H or lower alkyl can be alkylated on nitrogen, for example with benzylbromide in the presence of a suitable base, for example NaH in DMF at 0° C. to give a compound of structure XV. Treatment of XV with a dialkylcarbonate, such as dimethyl carbonate in the presence of a strong base, for example lithium diisopropylamide at a temperature between −78° C. and room temperature in a suitable inert solvent such as THF leads to the corresponding alkyl ester of structure XVI. Reduction of XVI with a strong reducing agent such as lithium aluminum hydride at a temperature of 0° C. to room temperature in a suitable solvent such as THF leads to an alcohol of structure XVII in which the hydroxyl moiety can be converted into a leaving group, for example by treatment with tosyl chloride in the present of a suitable base, for example triethylamine in dichloromethane to give a compound such as XVIII, which is suitable for use in the alkylation reaction described in Scheme 1. The alcohol XVII could also be converted into other leaving groups such as a halogen if use of a tosyl group is not desired.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

The structures of the examples were converted into a name using ChemDraw Ultra by PerkinElmer Informatics.

### Preparative HPLC:

Preparative purification by HPLC was carried out on a Waters 2707 Auto Purification system equipped with a 2998 PDA detector and using a X-Bridge C18, 250×30 mm ID, 5 column; mobile phase A: 0.01M aqueous ammonium acetate, mobile phase B: acetonitrile. The gradient program was set based on the selectivity and polarity of the compounds. Detection was set at 210 nm.

### NMR:

Proton NMR were recorded on an Agilent 400MRDD2 instrument with linux software.

### HPLC:

Analytical purity was determined on a Waters Acquity UPLC system with a 2998 PDA detector using an Acquity BEH C18, 100×2.1 mm, and 1.7μ column. Method 1 employed a mobile phase A of 0.025% aqueous TFA; mobile phase B of 0.025% TFA in acetonitrile and Method 2 employed a mobile phase A of 0.025% aqueous formic acid; mobile phase B of 0.025% formic acid in acetonitrile. Run times were 6 min with the gradients determined by compound polarity; the detection range was 210 to 400 nm.

### LCMS:

LC-MS was determined on Waters Acquity UPLC system with PDA detector, Using an Acquity; BEH; C18, 50×2.1 mm; 1.7μ Column. Method employed was a mobile phase A: 0.05% aqueous formic acid; mobile phase B: 0.05% formic acid in acetonitrile. The gradient program varied based on compound polarity over a 5 minutes run time and a detection range of 200 nm to 400 nm was employed. The MS detector used was a Waters Single Quadra pole Mass Detector, model SQD-2 with Z-spray technique equipped with an ESI source employing both ‘Positive’ and ‘Negative’ scan modes.

### Intermediate 1

### Synthesis of 1-(tetrahydro-2H-pyran-2-yl)-4-(4,4,5,5-tetramethyl-1,3,2-dioxaborolan-2-yl)-1H-pyrazole

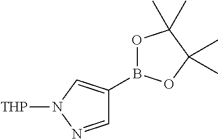

Step 1. A mixture of 4-bromo-1H-pyrazole (150 g, 1.02 mol, 1.0 eq), 3,4-dihydro-2H-pyran (128 g, 1.50 mol, 1.5 eq) and trifluoroacetic acid (7.8 mL, 0.10 mol, 0.1 eq) was stirred at 80° C. for 16 hr. Progress of the reaction was monitored by TLC (10% ethyl acetate-hexane R_{f}=0.4). After completion of reaction, the reaction mixture was diluted with ethyl acetate and washed with saturated aqueous sodium bicarbonate and brine. The organic layer was dried over anhydrous sodium sulfate, filtered and the solvents evaporated under reduced pressure to obtain 4-bromo-1-(tetrahydro-2H-pyran-2-yl)-1H-pyrazole (180 g, 76%) as a brown oil. LCMS purity: 81.4%; (ES⁺): m/z 231.2 (M+H⁺); tr=1.88 min.

Step 2. Bis(pinacolato)diboron (247 g, 0.974 mol, 1.5 eq) was added to a solution of 4-bromo-1-(tetrahydro-2H-pyran-2-yl)-1H-pyrazole (150 g, 0.65 mol, 1.0 eq) in 1,4-dioxane (1500 ml) at room temperature. Potassium acetate (127 g, 1.30 mol, 2 eq) was then added and the reaction flask was purged with argon for 20 min. PdCl₂(dppf) DCM (26.0 g, 31.8 mmol, 0.05 eq) was added and the mixture was purged with argon for further 10 min followed by stirring at 80° C. for 12 hr. After completion of the reaction (monitored by TLC, 10% ethyl acetate-hexane R_{f}=0.3), the mixture was cooled to room temperature and filtered through a bed of diatomaceous earth. The bed of diatomaceous earth was washed with ethyl acetate and the combined organic layers were evaporated under reduced pressure to give 1-(tetrahydro-2H-pyran-2-yl)-4-(4,4,5,5-tetramethyl-1,3,2-dioxaborolan-2-yl)-1H-pyrazole (280 g crude) as a brown oil. LCMS purity: 57.8%; (ES⁺): m/z 279.18 (M+H⁺); tr=1.95 min. The compound was used as such without further purification.

### Intermediate 2

### Synthesis of tert-butyl 4-(tosyloxymethyl) piperidine-1-carboxylate

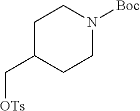 [SMILES: Cc1ccc(S(=O)(=O)OCC2CCN(C(=O)OC(C)(C)C)CC2)cc1]

To a solution of tert-butyl 4-(hydroxymethyl)piperidine-1-carboxylate (13.0 g, 60.4 mmol, 1.0 eq) and triethylamine (25.3 mL, 181 mmol, 3.0 eq) in dichloromethane (130 mL) was added TsCl (17.2 g, 90.0 mmol, 1.5 eq) slowly at 0° C. The mixture was allowed to stir at room temperature for 16 hr. After completion of the reaction (monitored by thin layer chromatography, 30% ethyl acetate in hexanes R_{f}=0.4), the mixture was poured into cold water and extracted with dichloromethane. The organic extract was washed with water followed by brine, dried over anhydrous sodium sulphate, filtered and concentrated under reduced pressure. The crude product was purified by column chromatography on silica gel (100-200 mesh), eluting with a 15-20% gradient of ethyl acetate in hexanes to afford tert-butyl 4-(tosyloxymethyl)piperidine-1-carboxylate (21 g) as a white solid. LCMS purity: 97.53%; (ES⁺): m/z 314 (M-56+H⁺); tr=2.35 min.

### Intermediate 3

### Synthesis of 5-bromo-1H-pyrrolo[2,3-c]pyridine

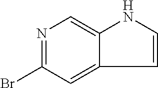

### Reaction Step 1. Synthesis of 2-(5-bromo-2-nitrophenyl)-N,N-dimethylethen-1-amine

A mixture of 1,1-dimethyoxy-N,N-dimethylmethanamine (65.0 g, 552 mmol, 4.0 eq) and 2-bromo-4-methyl-5-nitropyridine (30.0 g, 138 mmol, 1.0 eq) in DMF (200 mL) was heated to 100° C. and stirred for 4 hr. The mixture was concentrated under reduced pressure to give an off-white solid (38.0 g crude LCMS purity 30%. (ES⁺): m/z 273.24 (M+H⁺); tr=2.0 min.

### Reaction Step 2. Synthesis of 5-bromo-1H-pyrrolo[2,3-c]pyridine

The crude product from above was dissolved in acetic acid (1000 mL), iron (78.0 g, 1400 mmol, 10 eq) was added at room temperature and the mixture was heated to 70° C. for 2 hr, filtered through a celite pad and concentrated under reduced pressure. The residue was basified with saturated NaHCO₃ solution solid and the resulting solid was collected by filtration. The crude product was purified by column chromatography (100-200 silica gel) to give 5-bromo-1H-pyrrolo[2,3-c]pyridine as a brown solid (11.0 g). LCMS purity 98.8% (ES⁺): m/z 197.24 (M+H⁺); tr=1.3 min

### Intermediate 4

### Synthesis of tert-butyl 4-((5-bromo-1H-pyrrolo[2,3-c]pyridin-1-yl)methyl)piperidine-1-carboxylate

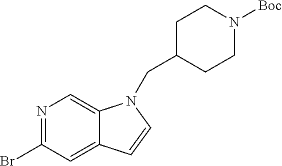 [SMILES: CC(C)(C)OC(=O)N1CCC(Cn2ccc3cc(Br)ncc32)CC1]

Sodium hydride (60% suspension in mineral oil, 1.53 g, 38 mmol, 1.5 eq) was added to a stirred solution of 5-bromo-1H-pyrrolo[2,3-c]pyridine (5.0 g, 25.0 mmol, 1.0 eq) in DMF (50 mL) at 0° C. and stirring was continued at room temperature for 30 min. The mixture was again cooled to 0° C. and a solution of tert-butyl 4-(tosyloxymethyl)piperidine-1-carboxylate (10.4 g, 28 mmol, 1.1 eq) in DMF (50 mL) was slowly added at 0° C. and stirring was continued at 65-70° C. for 16 hr. After completion of the reaction (monitored by thin layer chromatography, 30% ethyl acetate in hexanes, R_{f}=0.5), the reaction was quenched by adding ice cubes and the mixture was extracted with tert-butyl methyl ether (2×200 mL). The organic extract was dried over anhydrous sodium sulphate, filtered and concentrated under reduced pressure. The residue was purified by column chromatography on silica gel 100-200 mesh, eluting with a 25-40% gradient of ethyl acetate in hexanes to afford tert-butyl 4-((5-bromo-1H-pyrrolo[2,3-c]pyridin-1-yl)methyl)piperidine-1-carboxylate (9.0 g, 90%) as an off white solid. LCMS purity: 80%; (ES⁺): m/z 394.26 (M+H⁺).

### Intermediate 5

### Synthesis of 5-bromo-1-(piperidin-4-ylmethyl)-1H-pyrrolo[2,3-C]pyridine hydrochloride

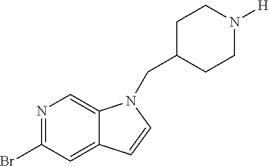 [SMILES: Brc1cc2ccn(CC3CCNCC3)c2cn1]

A solution 4N HCl in dioxane (40 mL) was slowly added to a solution of tert-butyl 4-((5-bromo-1H-pyrrolo[2,3-c]pyridin-1-yl)methyl)piperidine-1-carboxylate (9.0 g, 25.0 mmol) in dichloromethane (100 mL) at 0° C. and allowed to stir at room temperature for 2 hr. After completion of the reaction (monitored by thin layer chromatography, 10% methanol in dichloromethane R_{f}=0.2), the mixture was concentrated under reduced pressure and the crude product obtained was triturated with diethyl ether to obtain crude 5-bromo-1-(piperidin-4-ylmethyl)-1H-pyrrolo[2,3-c]pyridine hydrochloride (10 g) as an off white solid. LCMS purity: 75%; (ES⁺): m/z 294.28 (M+H⁺)

### Intermediate 6

### Synthesis of (4-((5-bromo-1H-pyrrolo[2,3-c]pyridin-1-yl)methyl)piperidin-1-yl)(phenyl)methanone

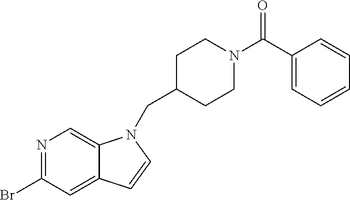 [SMILES: O=C(c1ccccc1)N1CCC(Cn2ccc3cc(Br)ncc32)CC1]

Benzoyl chloride (0.719 g, 5 mmol, 1.5 eq) was added to a stirred solution of 5-bromo-1-(piperidin-4-ylmethyl)-1H-pyrrolo[2,3-c]pyridine hydrochloride (1.0 g 3.4 mmol, 1.0 eq) and triethylamine (2.3 mL, 17 mmol, 5.0 eq) in dichloromethane (20 mL) at 0° C. and the mixture was stirred at room temperature for 1 hr. After completion of the reaction (monitored by thin layer chromatography, 3:1 ethyl acetate in pet-ether; R_{f}=0.3), chilled water was added and the mixture was extracted with dichloromethane. The organic extract was dried over anhydrous sodium sulphate, filtered and concentrated under reduced pressure. The crude product was purified by column chromatography on silica gel 100-200 mesh, eluting with an 80-85% gradient of ethyl acetate in hexanes to afford (4-((5-bromo-1H-pyrrolo[2,3-c]pyridin-1-yl)methyl)piperidin-1-yl)(phenyl)methanone (500 mg, 37%) as an off white semi-solid. (ES⁺): m/z 398.20 (M+H⁺); tr=2.05 min.

### Intermediates 7-13 Shown in the Table Below were Prepared Starting with Intermediate 5 and Using the Above Protocol with the Commercially Available Acid Chlorides Shown

| | Acid | | Yield | |
|---|---|---|---|---|
| Interm | Chloride | Name and Structure | (%) | LCMS |
| 7 | 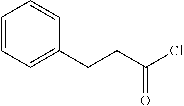 [SMILES: O=C(Cl)CCc1ccccc1] | 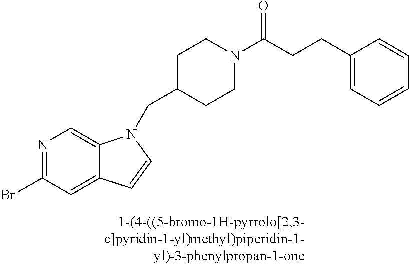 [SMILES: O=C(CCc1ccccc1)N1CCC(Cn2ccc3cc(Br)ncc32)CC1] | 57 | Purity: 75.53% (ES⁺): m/z 426.23 (M + H⁺); tr = 2.17 min. |
| 8 | 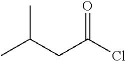 | 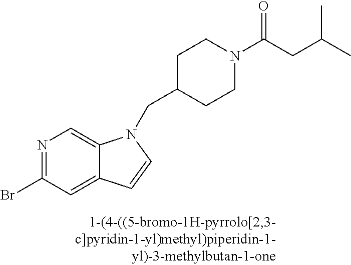 [SMILES: CC(C)CC(=O)N1CCC(Cn2ccc3cc(Br)ncc32)CC1] | 62 | Purity: 82.63% (ES⁺) m/z 378.2; (M + H⁺) tr = 2.07 min. |
| 9 | 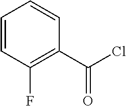 | 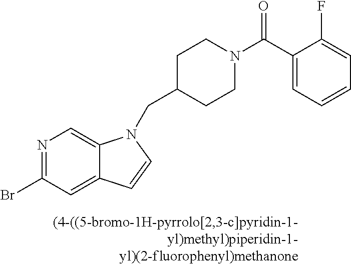 [SMILES: O=C(c1ccccc1F)N1CCC(Cn2ccc3cc(Br)ncc32)CC1] | 56 | Purity: 69% (ES⁺) m/z 416.18 (M + H⁺); tr = 2.05 min. |
| 10 | 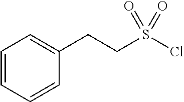 [SMILES: O=S(=O)(Cl)CCc1ccccc1] | 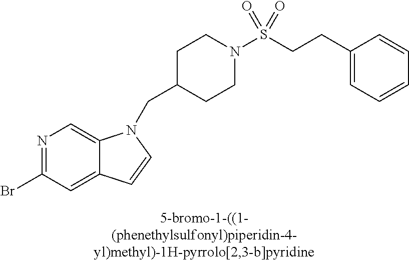 [SMILES: O=S(=O)(CCc1ccccc1)N1CCC(Cn2ccc3cc(Br)ncc32)CC1] | 57 | Purity 62.65% (ES⁺) m/z 462.23 (M + H⁺); tr = 2.29 min. |
| 11 | 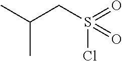 | 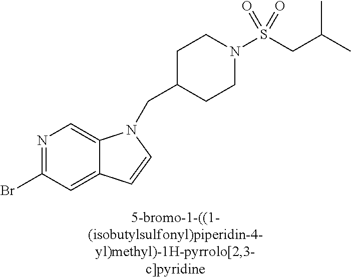 [SMILES: CC(C)CS(=O)(=O)N1CCC(Cn2ccc3cc(Br)ncc32)CC1] | 43 | Purity: 79% (ES⁺) m/z 414.34 (M + H⁺); tr = 2.19 min. |
| 12 | 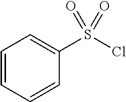 | 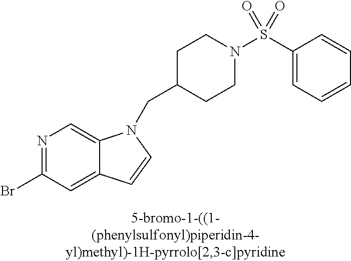 [SMILES: O=S(=O)(c1ccccc1)N1CCC(Cn2ccc3cc(Br)ncc32)CC1] | 41 | Purity: 76.75% (ES⁺) m/z 434.27 (M + H⁺); tr = 2.2 min. |
| 13 | 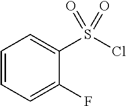 [SMILES: O=S(=O)(Cl)c1ccccc1F] | 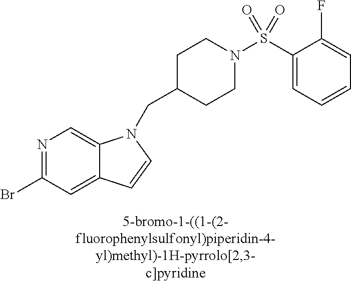 [SMILES: O=S(=O)(c1ccccc1F)N1CCC(Cn2ccc3cc(Br)ncc32)CC1] | 46 | Purity: 88.74% (ES⁺) m/z 452.16 (M + H⁺); tr = 2.23 min. |

### Intermediate 14

### Synthesis of tert-butyl 4-((5-bromo-1H-pyrrolo[2,3-b]pyridin-1-yl)methyl)piperidine-1-carboxylate

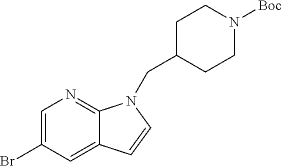 [SMILES: CC(C)(C)OC(=O)N1CCC(Cn2ccc3cc(Br)cnc32)CC1]

Sodium hydride (60% suspension in mineral oil, 1.52 g, 38 mmol 1.5 eq) was added to a stirred solution of 5-bromo-1H-pyrrolo[2,3-b]pyridine (5.0 g, 25.0 mmol, 1.0 eq) in DMF (50 mL) at 0° C. and the mixture was stirred at room temperature for 30 min. The mixture was again cooled to 0° C. and a solution of tert-butyl 4-(tosyloxymethyl)piperidine-1-carboxylate (12.2 g, 32.0 mmol, 1.3 eq) in DMF (50 mL) was slowly added at 0° C. Stirring was continued at 65-70° C. for 16 hr. After completion of the reaction (monitored by thin layer chromatography, 20% ethyl acetate in hexanes, R_{f}=0.5), the reaction was quenched by adding ice cubes and the resulting mixture was extracted with tert-butyl methyl ether (2×200 mL). The organic extract was dried over anhydrous sodium sulphate, filtered and concentrated under reduced pressure. The crude product was purified by column chromatography on silica gel 100-200 mesh, eluting with a 25-40% gradient of ethyl acetate in hexanes to afford tert-butyl 4-((5-bromo-1H-pyrrolo[2,3-b]pyridin-1-yl)methyl)piperidine-1-carboxylate (10.0 g, 100%) as an off white solid LCMS purity: 98.98%; (ES⁺): m/z 394.26 (M+H⁺); tr=2.56 min.

### Intermediate 15

### Synthesis of 5-bromo-1-(piperidin-4-ylmethyl)-1H-pyrrolo[2,3-b]pyridine hydrochloride

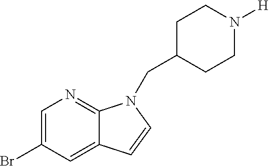 [SMILES: Brc1cnc2c(ccn2CC2CCNCC2)c1]

A solution of 4N hydrogen chloride in 1,4-dioxane (40 mL) was slowly added to a solution of tert-butyl 4-((5-bromo-1H-pyrrolo[2,3-b]pyridin-1-yl)methyl)piperidine-1-carboxylate (10.0 g, 25.0 mmol) in dichloromethane (100 mL) at 0° C. and the mixture was allowed to stir at room temperature for 2 hr. After completion of the reaction (monitored by thin layer chromatography, 10% methanol in dichloromethane R_{f}=0.2), the mixture was concentrated under reduced pressure and the crude product was triturated with diethyl ether to obtain 5-bromo-1-(piperidin-4-ylmethyl)-1H-pyrrolo[2,3-b]pyridine hydrochloride (8.3 g, 99%) as a Light brown solid. LCMS purity: 99.48%; (ES⁺): m/z 294.28 (M+H⁺); tr=1.27 min.

### Intermediate 16

### Synthesis of (4-((5-bromo-1H-pyrrolo[2,3-b]pyridin-1-yl)methyl)piperidin-1-yl)(phenyl)methanone

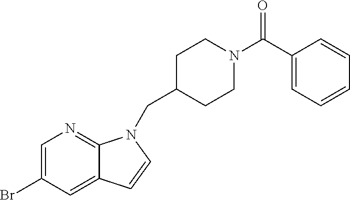 [SMILES: O=C(c1ccccc1)N1CCC(Cn2ccc3cc(Br)cnc32)CC1]

Benzoyl chloride (0.276 g, 1.9 mmol, 1.3 eq) was added to a stirred solution of 5-bromo-1-(piperidin-4-ylmethyl)-1H-pyrrolo[2,3-b]pyridine hydrochloride (0.5 g, 1.5 mmol, 1.0 eq) and triethylamine (1.05 mL, 7.5 mmol, 5.0 eq) in dichloromethane (20 mL) at 0° C. and the mixture was stirred at room temperature for 1 hr. After completion of the reaction (monitored by thin layer chromatography, 30% ethyl acetate in pet-ether; R_{f}=0.4), chilled water was added and the mixture was extracted with dichloromethane. The organic extract was dried over anhydrous sodium sulphate, filtered and concentrated under reduced pressure. The crude product was purified by column chromatography on silica gel 100-200 mesh, eluting with an 80-85% gradient of ethyl acetate in hexanes to afford (4-((5-bromo-1H-pyrrolo[2,3-b]pyridin-1-yl)methyl)piperidin-1-yl)(phenyl)methanone (500 mg, 83.3%) as a white solid. LCMS purity: 88.10%; (ES⁺): m/z 398.20 (M+H⁺); tr=2.27 min.

### Intermediates 17-23 Shown in the Table Below were Prepared Using the Above Protocol Starting with Intermediate 14 and the Acid Chloride Indicated

| | | | Yield | |
|---|---|---|---|---|
| Interm | Acid Chloride | Name and Structure | (%) | LCMS |
| 17 | 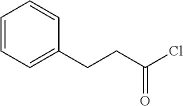 [SMILES: O=C(Cl)CCc1ccccc1] | 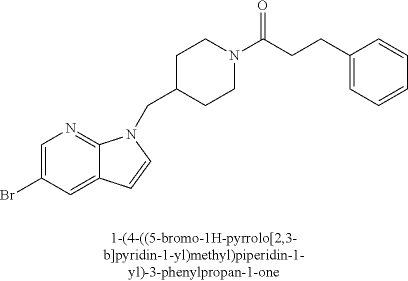 [SMILES: O=C(CCc1ccccc1)N1CCC(Cn2ccc3cc(Br)cnc32)CC1] | 71.5 | Purity: 97.37% (ES⁺): m/z 426.11 (M + H⁺): tr = 2.46 min. |
| 18 | 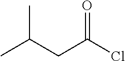 | 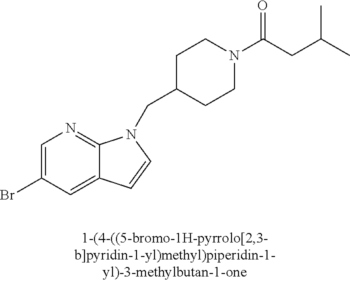 [SMILES: CC(C)CC(=O)N1CCC(Cn2ccc3cc(Br)cnc32)CC1] | 87.7 | Purity: 99.85% (ES⁺) m/z 378.2; tr = 2.29 min. |
| 19 | 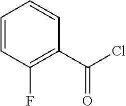 | 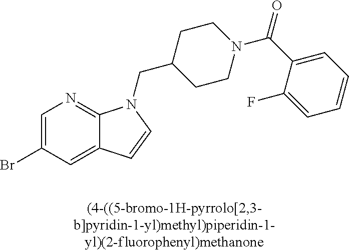 [SMILES: O=C(c1ccccc1F)N1CCC(Cn2ccc3cc(Br)cnc32)CC1] | 80.9 | Purity: 92.94% (ES⁺) m/z 418.18 (M + H⁺); tr = 2.3 min. |
| 20 | 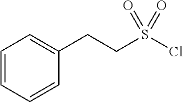 [SMILES: O=S(=O)(Cl)CCc1ccccc1] | 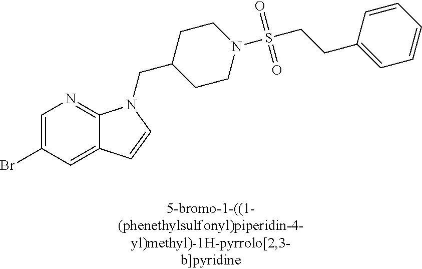 [SMILES: O=S(=O)(CCc1ccccc1)N1CCC(Cn2ccc3cc(Br)cnc32)CC1] | 72.6 | Purity: 97.37% (ES⁺) m/z 464.23 (M + H⁺); tr = 2.46 min. |
| 21 | 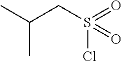 | 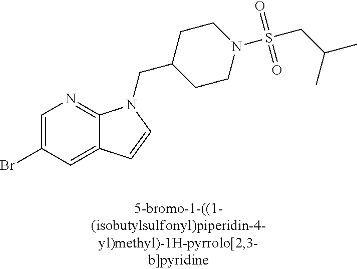 [SMILES: CC(C)CS(=O)(=O)N1CCC(Cn2ccc3cc(Br)cnc32)CC1] | 82.2 | Purity: 98.57% (ES⁺) m/z 414.34 (M + H⁺); tr = 2.39 min. |
| 22 | 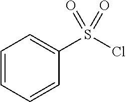 | 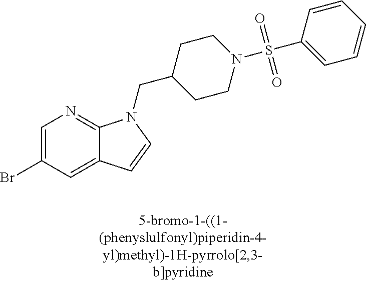 [SMILES: O=S(=O)(c1ccccc1)N1CCC(Cn2ccc3cc(Br)cnc32)CC1] | 79.2 | Purity: 98.86% (ES⁺) m/z 434.27 (M + H⁺); tr = 2.43 min. |
| 23 | 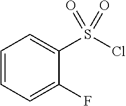 [SMILES: O=S(=O)(Cl)c1ccccc1F] | 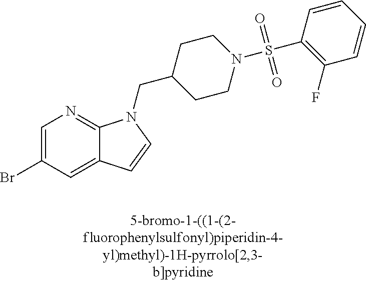 [SMILES: O=S(=O)(c1ccccc1F)N1CCC(Cn2ccc3cc(Br)cnc32)CC1] | 80.0 | Purity: 99.53% (ES⁺) m/z 454.16 (M + H⁺); tr = 2.43 min. |

### Intermediate 24

### Synthesis of 4-bromo-5-methyl-1-(tetrahydro-2H-pyran-2-yl)-1H-pyrazole

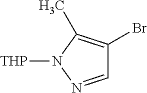

TFA (0.003 g, 0.03 mmol, 0.1 eq) was added to a solution of 4-bromo-5-methyl-1H-pyrazole (0.5 g, 3.0 mmol, 1.0 eq) in dihydropyran (0.525 g, 6.0 mmol, 2.0 eq) at 0° C. and the mixture was allowed to stir at 60° C. for 6 hr. After completion of the reaction (monitored by TLC, 50% ethyl acetate in hexanes R_{f}=0.7), the mixture was poured into cold water and extracted with EtOAc. The organic later was washed with sat. NaHCO₃ soln., water and brine, dried over anhydrous sodium sulfate, filtered and concentrated under reduced pressure to afford 4-bromo-5-methyl-1-(tetrahydro-2H-pyran-2-yl)-1H-pyrazole (0.6 g, 78.4%) as a brown liquid. LCMS purity: 92.54% (mixture of isomers: 65.43% and 27.11%); (ES⁺): m/z 245.09 (M+H⁺); tr=1.90 and 1.95 min.

### Intermediate 25

### Synthesis of 4-bromo-1-(tetrahydro-2H-pyran-2-yl)-5-(trifluoromethyl)-1H-pyrazole

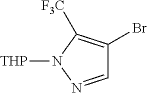

Reaction step 1. Synthesis of 5-(trifluoromethyl)-1H-pyrazole.

To a solution of (E)-4-ethoxy-1,1,1-trifluorobut-3-en-2-one (5.0 g, 30 mmol, 1.0 eq) in ethanol (50 mL) was added triethylamine (6.2 mL, 44 mmol, 1.5 eq) at 0° C. Hydrazine hydrochloride (3.3 g, 45 mmol, 1.5 eq) was added to the reaction mixture. The reaction mixture was allowed to stir at 80° C. for 12 hr. After completion of the reaction (monitored by TLC, 15% ethyl acetate in hexanes R_{f}=0.2), the mixture was concentrated under reduced pressure and the residue was poured into cold water and extracted with EtOAc. The organic extract was washed with water followed by brine, dried over anhydrous sodium sulfate, filtered and concentrated under reduced pressure to afford crude 5-(trifluoromethyl)-1H-pyrazole (3.74 g) as a brown liquid, which was used as such in the next step. LCMS purity: (54.69)%; (ES⁺): m/z 137.0 (M+H⁺); tr=1.38 min.

Reaction step 2. Synthesis of 4-bromo-5-(trifluoromethyl)-1H-pyrazole.

To a solution 5-(trifluoromethyl)-1H-pyrazole (3.70 g crude, 15 mmol as per LCMS purity of crude, 1.0 eq) in acetonitrile (40 mL) was added N-bromosuccinamide (5.81 g, 32.6 mmol, 2.19 eq) at 0° C. and the mixture was allowed to stir at room temperature for 8 hr. After completion of the reaction (monitored by TLC, 30% ethyl acetate in hexanes, R_{f}=0.5), the reaction mixture was poured into cold water and extracted with EtOAc. The organic extract was washed with water followed by brine, dried over anhydrous sodium sulfate, filtered and concentrated under reduced pressure to afford crude 4-bromo-5-(trifluoromethyl)-1H-pyrazole (5.82 g) which was used as such in the next step. LCMS purity: 57.85%; (ES⁺): m/z 214.98 (M+H⁺); tr=1.70 min.

Reaction step 3. Synthesis of 4-bromo-1-(tetrahydro-2H-pyran-2-yl)-5-(trifluoromethyl)-1H-pyrazole

To a solution of 4-bromo-5-(trifluoromethyl)-1H-pyrazole (5.80 g, 15.6 mmol as per LCMS purity of crude, 1.0 eq) in ethyl acetate (60 mL), dihydropyran (4.55 g, 54.1 mmol, 3.5 eq) and p-toluenesulfonic acid (0.466 g, 0.27 mmol, 0.1 eq) was added at 0° C. and the mixture was allowed to stir at 80° C. for 4 hr. After completion of the reaction (monitored by TLC, 20% ethyl acetate in hexanes R_{f}=0.5), the mixture was poured into cold water and extracted with EtOAc. The organic extract was washed with sat. NaHCO₃ soln. followed by brine, dried over anhydrous sodium sulfate, filtered and concentrated under reduced pressure to afford crude 4-bromo-1-(tetrahydro-2H-pyran-2-yl)-5-(trifluoromethyl)-1H-pyrazole (7.23 g) as a brown liquid, which was used as such in the next step. LCMS purity: 45.5%; (ES⁺): m/z 299.5 (M+H⁺); tr=2.27 min.

### Intermediate 26

### Synthesis of benzyl 3-(tosyloxymethyl)pyrrolidine-1-carboxylate

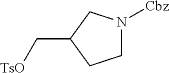 [SMILES: Cc1ccc(S(=O)(=O)OCC2CCN(C(=O)OCc3ccccc3)C2)cc1]

To a solution of benzyl 3-(hydroxymethyl)pyrrolidine-1-carboxylate (7.50 g, 31.9 mmol, 1.0 eq) and triethylamine (13 mL, 95.7 mmol, 3.0 eq) in dichloromethane (70 mL) was added TsCl (9.12 g, 47.8 mmol, 1.5 eq) slowly at 0° C. The mixture was allowed to room temperature and was stirred overnight. After completion of the reaction (monitored by TLC, 50% ethyl acetate-hexane R_{f}=0.6), the mixture was poured into cool water and washed with water followed by brine. The organic layer was dried over anhydrous sodium sulfate and solvent was removed under reduced pressure. The crude product was purified by column chromatography over neutral alumina, eluting with a 0-12% gradient of ethyl acetate in hexanes to afford benzyl 3-(tosyloxymethyl)pyrrolidine-1-carboxylate (8.16 g, 68%) as a colourless oil. LCMS m/z=390 (M+1.

### Intermediate 27

### Synthesis of 1-benzyl-5-methylpyrrolidin-3-yl)methyl 4-methylbenzenesulfonate

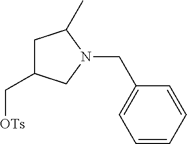 [SMILES: Cc1ccc(S(=O)(=O)OCC2CC(C)N(Cc3ccccc3)C2)cc1]

Reaction step 1. Synthesis of 1-benzyl-5-methylpyrrolidin-2-one:

To a solution of 5-methylpyrrolidin-2-one (15 g, 152 mmol, 1.0 eq) in DMF (115 mL), was slowly added NaH (5.4 g, 230 mmol, 1.5 eq) followed by benzyl bromide (21.7 mL, 182 mmol, 1.2 eq) at 0° C. and the reaction mixture was allowed to warm to room temperature over 3 hr. After completion of reaction (monitored by TLC, 20% ethyl acetate-hexane, KMnO₄, R_{f}=0.45), the reaction was quenched by the addition of ice cubes and was extracted with ethyl acetate (500 mL). The organic extract was dried over anhydrous sodium sulfate and the solvent removed under reduced pressure. The crude product was purified by column chromatography on silica gel (100-200 mesh), eluting with 10% ethyl acetate in hexanes to afford 25 g of 1-benzyl-5-methylpyrrolidin-2-one as oil. LC-MS (ES⁺) m/z: 190.1 (M+1); purity=92.5%.

Reaction step 2. Synthesis of methyl 1-benzyl-5-methyl-2-oxopyrrolidine-3-carboxylate:

To a solution of diisopropyl amine (7.84 mL, 55.5 mmol, 2.1 eq) in THF (50 mL), at −78° C. was slowly added n-BuLi (2.5 M in hexanes) (21.5 mL, 52.8 mmol, 2.0 eq) and the mixture was allowed to warm to −20° C. for 40 min. A solution of 1-benzyl-5-methylpyrrolidin-2-one (5.0 g, 26 mmol, 1.0 eq) in THF was added to the above reaction mixture at −78° C. and the mixture was stirred for 45 min. Then dimethyl carbonate (4.45 mL, 52.8 mmol, 2.0 eq) was added at −78° C. and the mixture was allowed to warm to room temperature slowly over 5 hr. After completion of the reaction (monitored by TLC, 30% ethyl acetate-hexane, KMnO₄, R_{f}=0.65), the reaction was quenched by the slow addition of 1M HCl at 0° C. and the mixture was extracted with ethyl acetate (300 mL). The organic extract was dried over anhydrous sodium sulfate and the solvents were removed under reduced pressure to afford 2.1 g of methyl 1-benzyl-5-methyl-2-oxopyrrolidine-3-carboxylate as a sticky foam. LC-MS (ES⁺) m/z: 248.1 (M+1); purity=92% (mixture of isomers).

Reaction step 3. Synthesis of (1-benzyl-5-methylpyrrolidin-3-yl) methanol:

Lithium Aluminium hydride (2M in hexane, 15.7 mL, 31.5 mmol, 3.7 eq) was added slowly to a solution of methyl 1-benzyl-5-methyl-2-oxopyrrolidine-3-carboxylate (2.1 g, 8.20 mmol, 1.0 eq) in THF (35 mL), at 0° C. and the mixture was allowed to warm to room temperature over 3 hr. After completion of the reaction (monitored by TLC, 30% ethyl acetate-hexane, KMnO₄, R_{f}=0.45), the reaction was quenched by slow addition of 1.25 mL of water and 1.25 mL of 15% NaOH solution followed by 3.75 mL of water at 0° C. The reaction mixture was filtered through a small bed of celite and the filtrate was concentrated under reduced pressure to afford 1.7 g (crude) of (1-benzyl-5-methylpyrrolidin-3-yl) methanol as sticky foam. LC-MS (ES⁺) m/z: 206.1 (M+1); purity=80% (mixture of isomers).

Reaction step 4. Synthesis of (1-benzyl-5-methylpyrrolidin-3-yl)methyl 4-methylbenzenesulfonate:

To a solution of (1-benzyl-5-methylpyrrolidin-3-yl)methanol (1.7 g, 8.3 mmol, 1.0 eq) in dichloromethane (25 mL), triethylamine (3.47 mL, 24.9 mmol, 3.0 eq) was added, followed by tosyl chloride (1.89 g, 10 mmol, 1.2 eq) at 0° C. The reaction mixture was allowed to warm to room temperature and was stirred for 12 hr. After completion of the reaction (monitored by TLC, 50% ethyl acetate-hexane, R_{f}=0.65), the reaction mixture was quenched by addition of NaHCO₃ solution (25 mL) and was extracted with dichloromethane. The combined extracts were washed with brine solution. The organic layer was dried over anhydrous sodium sulfate and concentrated under reduced pressure to afford 0.92 g (31%) of (1-benzyl-5-methylpyrrolidin-3-yl)methyl 4-methylbenzenesulfonate as a sticky solid. LC-MS (ES⁺) m/z: 360.16 (M+1); purity=75%.

### Intermediate 28

### Benzyl 4-((tosyloxy)methyl)azepane-1-carboxylate

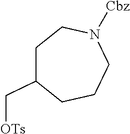 [SMILES: Cc1ccc(S(=O)(=O)OCC2CCCN(C(=O)OCc3ccccc3)CC2)cc1]

Reaction step 1. Synthesis of 1-benzyl 4-ethyl-5-oxoazepane-1,4-dicarboxylate.

Ethyl diazoacetate (12.7 mL, 112 mmol, 1.3 eq) was added to a solution of benzyl 4-oxopiperidine-1-carboxylate (20.0 g, 85.8 mmol, 1.0 eq) in diethyl ether (200 mL) at −78° C. followed by and BF₃.OEt₂ (4.4 mL, 86 mmol, 1.0 eq). The reaction mixture was stirred at −78° C. for 1 hr then allowed to attain to room temperature to give a clear solution. After completion of reaction (monitored by TLC, 20% ethyl acetate-hexane R_{f}=0.5), a saturated solution of K₂CO₃ was added to the reaction mixture and the organic layer was separated and washed with saturated K₂CO₃ solution. The organic layer was dried over anhydrous sodium sulfate and concentrated under reduced pressure. The crude product was purified by flash column chromatography on silica gel (100-200 mesh), eluting with 5% ethyl acetate in hexanes to afford 1-benzyl 4-ethyl 5-oxoazepane-1,4-dicarboxylate as a colorless oil. Yield=13.0 g, 48% LCMS m/z=320.25 (M+1); purity=>90% by ¹H NMR.

Reaction step 2. Synthesis of 1-benzyl 4-ethyl 5-hydroxyazepane-1,4-dicarboxylate.

Sodium borohydride (1.5 g, 40.8 mmol, 1.0 eq) was added portion wise to a solution of 1-benzyl 4-ethyl 5-oxoazepane-1,4-dicarboxylate (13.0 g, 40.8 mmol, 1.0 eq) in EtOH (130 mL) at 0° C. The reaction mixture was stirred at 0° C. for 1 hr. After completion of the reaction (monitored by TLC, 30% ethyl acetate-hexane R_{f}=0.3), the reaction was quenched by addition of a saturated aqueous solution of potassium sodium tartrate and the solid was filtered. The filtrate was diluted with dichloromethane and washed with a saturated solution of aqueous potassium sodium tartrate followed by water. The organic layer was dried over anhydrous sodium sulfate and concentrated under reduced pressure to give 10.0 g of crude 1-benzyl 4-ethyl 5-hydroxyazepane-1,4-dicarboxylate that was used in the next step without purification or characterization.

Reaction step 3. Synthesis of 1-benzyl 4-ethyl 2,3,6,7-tetrahydro-1H-azepine-1,4-dicarboxylate.

To a solution of 1-benzyl 4-ethyl 5-hydroxyazepane-1,4-dicarboxylate (10.0 g, 31.0 mmol, 1.0 eq) in THF (100 mL) and triethylamine (12.6 mL 93.0 mmol, 3.0 eq) at 0° C., methanesulfonyl chloride (5.9 mL, 78 mmol, 2.5 eq) was added in three portions over 6 hr. After completion of the reaction (monitored by TLC, 20% ethyl acetate-hexane R_{f}=0.3), the reaction mixture was diluted with dichloromethane and washed with saturated aqueous NaHCO₃. The organic layer was dried over anhydrous sodium sulfate and concentrated under reduced pressure. The residue (20 g) was dissolved in THF (10 vol), DBU (11.4 mL, 46.5 mmol, 1.5 eq) was added and the reaction mixture and heated 80° C. for 1 hr. After completion of the reaction (monitored by TLC, 20% ethyl acetate-hexane R_{f}=0.6), the reaction mixture was diluted with dichloromethane and washed with saturated aqueous NaHCO₃. The organic layer was dried over anhydrous sodium sulfate and concentrated under reduced pressure. The crude product was purified by flash column chromatography on silica gel (100-200 mesh), eluting with 10% ethyl acetate in hexanes to afford 1-benzyl 4-ethyl 2,3,6,7-tetrahydro-1H-azepine-1,4-dicarboxylate (6.5 g, 80%) as a colorless oil. LCMS m/z=304.16 (M+1); purity=91%.

Reaction step 4. Synthesis of benzyl 4-(hydroxymethyl)azepane-1-carboxylate.

LiBH₄ (0.80 g, 36.3 mmol, 2.0 eq) was added to a solution of 1-benzyl 4-ethyl 2,3,6,7-tetrahydro-1H-azepine-1,4-dicarboxylate (5.50 g, 18.2 mmol, 1 eq) in THF (55 mL) at 0° C. in three portions over 30 min. The reaction mixture was heated to 60° C. for 6 hr. After completion of the reaction (monitored by TLC, 20% ethyl acetate-hexane (R_{f}=0.2), the reaction mixture was cooled to 0° C., quenched with ice cold water and extracted with ethyl acetate. The organic layer was dried over anhydrous sodium sulfate and concentrated under reduced pressure. The crude product was purified by flash column chromatography on silica gel (100-200 mesh), eluting with a 0-20% gradient of ethyl acetate in hexanes to obtain benzyl 4-(hydroxymethyl)azepane-1-carboxylate (2.80 g, 60%) as a colourless oil. LCMS m/z=264.25 (M+1), purity=95%.

Reaction step 5. Synthesis of benzyl 4-(tosyloxymethyl)azepane-1-carboxylate.

To a solution of benzyl 4-(hydroxymethyl)azepane-1-carboxylate (2.80 g, 10.6 mmol, 1 eq) in dichloromethane (28 mL) and triethylamine (4.3 mL, 31.9 mmol, 3.0) eq at 0° C., tosyl chloride (3.0 g, 15.7 mmol, 1.5 eq) was added and the reaction mixture was stirred overnight at room temperature. After completion of the reaction (monitored by TLC, 20% ethyl acetate-hexane (R_{f}=0.4), the reaction mixture was poured into ice cold water and extracted with dichloromethane. The organic extract was dried over anhydrous sodium sulfate and the solvents were removed under reduced pressure. The crude product was purified by flash column chromatography on silica gel (100-200 mesh), eluting with a 0-10% gradient of ethyl acetate in hexanes to obtain benzyl 4-(tosyloxymethyl)azepane-1-carboxylate as a colourless oil (2.30 g, 63%). LCMS m/z=418.19 (M+1); purity=96.2%.

### Intermediate 29

### Synthesis of rac.-benzyl 2-((tosyloxy)methyl)-7-azabicyclo[2.2.1]heptane-7-carboxylate

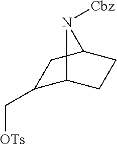 [SMILES: Cc1ccc(S(=O)(=O)OCC2CC3CCC2N3C(=O)OCc2ccccc2)cc1]

Reaction step 1. Synthesis of ethyl 3-bromopropiolate.

Silver nitrite (1.72 g, 10.2 mmol, 0.1 eq) was added to a solution of ethyl propiolate (10.0 g, 102 mmol, 1.0 eq) in acetone (200 mL) at room temperature. The resulting reaction mixture was stirred for 5 min, then NBS (20.0 g, 112 mmol, 1.1 eq) was added and the reaction mixture stirred for 2 hr at room temperature. After completion of the reaction (monitored by TLC, 5% ethyl acetate-hexane, R_{f}=0.55), the reaction mixture was filtered through a celite pad, washing with acetone. The filtrate was concentrated under reduced temperature (25-30° C.) to afford an oil. The crude product was purified by flash column chromatography on silica gel (100-200 mesh), eluting with 10% diethyl ether in hexanes to afford ethyl 3-bromopropiolate (10.0 g, 58%) as a yellow oil. LCMS m/z=176.91 (M+1).

Reaction step 2. Synthesis of 7-tert-butyl 2-ethyl 3-bromo-7-azabicyclo[2.2.1]hepta-2, 5-diene-2,7-dicarboxylate.

A mixture of methyl 3-bromopropiolate (5.00 g, 28.2 mmol, 1.0 eq) and tert-butyl 1H-pyrrole-1-carboxylate (14.00 g, 84.7 mmol, 3.0 eq) in a sealed tube was heated to 90° C. for 14 hr. After completion of the reaction (monitored by TLC, 5% ethyl acetate-hexane, R_{f}⁼0.3), the reaction mixture was purified without work up by flash column chromatography on silica gel (100-200 mesh), eluting with 5% ethyl acetate in hexanes to afford 7-tert-butyl 2-methyl 3-bromo-7-azabicyclo[2.2.1]hepta-2,5-diene-2,7-dicarboxylate (2.0 g, 20%) as a brown oil. LCMS m/z=344.2 (M+1); purity=75%.

Reaction step 3. Synthesis of 7-tert-butyl 2-methyl 7-azabicyclo[2.2.1]heptane-2,7-dicarboxylate.

To a stirred solutions of 7-tert-butyl 2-methyl 3-bromo-7-azabicyclo[2.2.1]hepta-2,5-diene-2,7-dicarboxylate (15.0 g, 43.7 mmol, 1.0 eq) in ethanol (300 mL), was added palladium on carbon (2.0 g) and the reaction mixture was stirred at room temperature for 3 hr under a hydrogen atmosphere maintained by a hydrogen filled balloon. After completion of the reaction (monitored by TLC, 10% ethyl acetate-hexane R_{f}=0.5), the mixture was filtered through a celite pad, washing with methanol. The filtrate was evaporated under reduced pressure to obtain 7-tert-butyl 2-ethyl 7-azabicyclo[2.2.1]heptane-2,7-dicarboxylate (9.90 g, 85%) as a light brown oil. LCMS m/z=255.25 (M-14) purity by ¹H NMR>90%.

Reaction step 4. Synthesis of ethyl 7-azabicyclo[2.2.1]heptane-2-carboxylate

A solution of 4M HCl in dioxane (100 mL, 400 mmol, 3.0 eq) was slowly added to a stirred solution of 7-tert-butyl 2-ethyl 3-bromo-7-azabicyclo[2.2.1]heptane-2,7-dicarboxylate (20.0 g, 74.3 mmol, 1.0 eq) in dioxane (400 mL) at 0° C. and the reaction mixture was stirred at room temperature for 2 hr. After completion of the reaction (monitored by TLC, 30% ethyl acetate-hexane, R_{f}=0.01), solvent was removed under reduced pressure and the residue was dried under vacuum to afford ethyl 7-azabicyclo[2.2.1]heptane-2-carboxylate HCl (12.0 g, 96%) as a yellow sticky mass. LCMS m/z=156.12 (M+1); crude purity by ¹H NMR ˜90%.

Reaction step 5. Synthesis of 7-benzyl 2-ethyl 7-azabicyclo[2.2.1]heptane-2,7-dicarboxylate.

To a stirred solution of ethyl 7-azabicyclo[2.2.1]heptane-2-carboxylate (12.0 g, 71.0 mmol, 1.0 eq) in dichloromethane (120 mL) was added triethylamine (25.9 mL, 355 mmol, 5.0 eq) at 0° C., and then benzyl chloroformate (13.3 g, 78.1 mmol, 1.1 eq) was slowly added. The mixture was stirred for 14 h at room temperature. After completion of the reaction (monitored by TLC, 30% ethyl acetate-hexane R_{f}=0.7), the reaction was quenched with saturated sodium bicarbonate solution, the product extracted with dichloromethane and the solvent was concentrated. The crude product was purified by flash column chromatography on silica gel (100-200 mesh), eluting with 10% ethyl acetate in hexane to obtain 7-benzyl 2-ethyl 7-azabicyclo[2.2.1]heptane-2,7-dicarboxylate (11.8 g, 58%) as a yellow oil. LCMS m/z=290.18 (M+1).

Reaction step 6. Synthesis of benzyl 2-(hydroxymethyl)-7-azabicyclo[2.2.1]heptane-7-carboxylate.

Lithium borohydride (5.50 g, 264 mmol, 4.0 eq) was slowly added to a stirred solution of 7-benzyl 2-ethyl 7-azabicyclo[2.2.1]heptane-2,7-dicarboxylate (20.0 g, 66.0 mmol, 1.0 eq) in tetrahydrofuran (400 mL) at 0° C. and the mixture was stirred at 60° C. for 12 hr. After completion of the reaction (monitored by TLC, 30% ethyl acetate-hexanes, R_{f}=0.15), the mixture was quenched with ice cold water and the product was extracted with ethyl acetate. The organic layer was dried over anhydrous sodium sulfate and concentrated to afford benzyl 2-(hydroxymethyl)-7-azabicyclo[2.2.1]heptane-7-carboxylate (13.6 g, 80%) as a light yellow oil. LCMS m/z=262.12 (M+1).

Reaction step 7. Synthesis of benzyl 2-(tosyloxymethyl)-7-azabicyclo[2.2.1]heptane-7-carboxylate.

To a stirred solution of benzyl 2-(hydroxymethyl)-7-azabicyclo[2.2.1]heptane-7-carboxylate (16.0 g, 61.3 mmol, 1.0 eq) in dichloromethane (160 mL) was added triethylamine (42.7 mL, 306 mmol, 5.0 eq) at 0° C., followed by the slow addition of tosyl chloride (17.4 g, 91.9 mmol, 1.5 eq). The reaction mixture was stirred for 14 hr at room temperature. After completion of the reaction (monitored by TLC, 30% ethyl acetate-hexane R_{f}=0.7), the reaction mixture was diluted with dichloromethane and washed with saturated sodium bicarbonate solution. The organic layer was dried over anhydrous sodium sulfate and concentrated under reduced pressure. The crude mass was purified by flash column chromatography on silica gel (100-200 mesh), eluting with 15% ethyl acetate in hexanes to obtain benzyl 2-(tosyloxymethyl)-7-azabicyclo[2.2.1]heptane-7-carboxylate (16.0 g, 64%) as an off white solid. LCMS m/z=416.14 (M+1).

### Intermediate 30

### Synthesis of (1-benzyl-5,5-dimethylpyrrolidin-3-yl)methyl 4-methylbenzenesulfonate

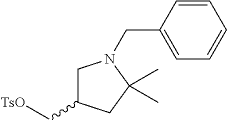 [SMILES: Cc1ccc(S(=O)(=O)OCC2CN(Cc3ccccc3)C(C)(C)C2)cc1]

Reaction step 1: Synthesis of 1-benzyl-5,5-dimethylpyrrolidin-2-one.

To a solution of 5,5-dimethylpyrrolidin-2-one (35.0 g, 310 mmol, 1.0 eq) in DMF (350 mL), NaH (60% suspension in paraffin oil, 18.6 g, 465 mmol, 1.5 eq) was slowly added followed by benzyl bromide (44.0 mL, 372 mmol, 1.2 eq) at 0° C., the mixture was allowed to warm to room temperature with continuous stirring and stirred at room temperature for 16 hr. After completion of the reaction (monitored by TLC, 50% ethyl acetate-hexane, R_{f}=0.50), reaction mixture was quenched by the addition of ice cubes and extracted with ethyl acetate (500 mL). The organic extract was dried over anhydrous sodium sulfate, filtered and concentrated under reduced pressure. The crude product was purified by column chromatography on silica gel (100-200 mesh), eluting with a 40% gradient of ethyl acetate in hexanes to afford 1-benzyl-5,5-dimethylpyrrolidin-2-one (40.0 g, 63.6%) as colourless viscous liquid. LCMS purity: 90.58%; (ES⁺): m/z 204.2 (M+H⁺); tr=1.77 min.

Reaction step 2. Synthesis of methyl 1-benzyl-5,5-dimethyl-2-oxopyrrolidine-3-carboxylate.

A stirred solution of diisopropyl amine (63.0 mL, 394 mmol, 2.0 eq) in THF (400 mL) was cooled to −78° C. and n-BuLi (2.5M in hexanes, 164.0 mL, 394 mmol, 2.0 eq) was slowly added. The mixture was allowed to warm to −20° C. and was stirred at −20° C. for 90 min. The mixture was then again cooled to −78° C. and a solution of 1-benzyl-5,5-dimethylpyrrolidin-2-one (40.0 g, 197 mmol, 1.0 eq) in THF (100 mL) was added slowly to the above mixture. Stirring was continued at −78° C. for 60 min followed by addition of dimethyl carbonate (36.0 mL, 413 mmol, 2.1 eq) slowly, while maintaining the temperature at −78° C. After completion of the addition, the mixture was allowed to warm to room temperature over 4 hr. After completion of the reaction (monitored by TLC, 50% ethyl acetate-hexanes, R_{f}=0.55), the reaction mixture was quenched by the slow addition of 1M HCl at 0° C. and the mixture was extracted with ethyl acetate. The organic extract was dried over anhydrous sodium sulfate, filtered and concentrated under reduced pressure to afford methyl 1-benzyl-5,5-dimethyl-2-oxopyrrolidine-3-carboxylate (20.0 g, 40%) as colorless viscous liquid. LCMS purity: 90.0%; (ES⁺): m/z 262.01 (M+H⁺); tr=1.82 min.

Reaction step 3. Synthesis of (1-benzyl-5,5-dimethylpyrrolidin-3-yl)methanol.

Lithium aluminium hydride (2M in hexane, 145 mL, 306 mmol, 4.0 eq) was added slowly to a stirred solution of methyl 1-benzyl-5,5-dimethyl-2-oxopyrrolidine-3-carboxylate (20.0 g, 76.6 mmol, 1.0 eq) in THF (200 mL) at 0° C. and stirring was continued while the mixture was allowed to warm up to room temperature over a period of 6 hr. After completion of the reaction (monitored by TLC, 30% ethyl acetate-hexanes, R_{f}=0.25), the reaction was quenched by the slow addition of 20 mL of water and 20 mL of 15% aqueous NaOH followed by 40 mL of water at 0° C. The precipitated solid was removed by filtering the mixture through a bed of celite and the filtrate was concentrated under reduced pressure. The crude product was purified by column chromatography on silica gel (100-200 mesh), eluting with 40% ethyl acetate in hexanes to afford (1-benzyl-5,5-dimethylpyrrolidin-3-yl)methanol (9.0 g, 53.8%) as colorless gel. LCMS purity: 89.15%; (ES⁺): m/z 220.30 (M+H⁺); tr=3.11 min.

Reaction step 4. Synthesis of (1-benzyl-5,5-dimethylpyrrolidin-3-yl)methyl 4-methylbenzenesulfonate.

To a solution of (1-benzyl-5,5-dimethylpyrrolidin-3-yl)methanol (5.0 g 23 mmol, 1.0 eq) in dichloromethane (50 mL), triethylamine (9.3 mL, 68 mmol, 3.0 eq) was added p-toluene sulfonyl chloride (5.2 g, 27.4 mmol, 1.2 eq) at 0° C. and the reaction mixture was stirred at room temperature for 12 hr. After completion of the reaction (monitored by TLC, 50% ethyl acetate-hexane, R_{f}=0.65), the reaction mixture was poured into ice-cold water and extracted with dichloromethane. The organic extract was washed with brine, dried over anhydrous sodium sulfate, filtered and concentrated under reduced pressure. The crude product was purified by column chromatography on silica gel (100-200 mesh), eluting with a 10-15% gradient of ethyl acetate in hexanes to afford (1-benzyl-5,5-dimethylpyrrolidin-3-yl)methyl 4-methylbenzenesulfonate (5.8 g, 68%) as colorless gel. LCMS purity: 78.3%; (ES⁺): m/z 374.32 (M+H⁺); tr=4.28 min.

### Intermediate 31

### Synthesis of of 1-(piperidin-4-ylmethyl)-5-(1-(tetrahydro-2H-pyran-2-yl)-1H-pyrazol-4-yl)-1H-pyrrolo [2,3-c]pyridine

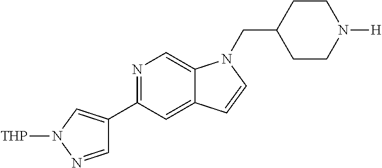

1-(Piperidin-4-ylmethyl)-5-(1-(tetrahydro-2H-pyran-2-yl)-1H-pyrazol-4-yl)-1H-pyrrolo[2,3-c]pyridine can be prepared in two steps from tert-butyl 4-((5-bromo-1H-pyrrolo[2,3-c]pyridin-1-yl)methyl)piperidine-1-carboxylate (Intermediate 4) by palladium catalyzed coupling with 1-(tetrahydro-2H-pyran-2-yl)-4-(4,4, 5,5-tetramethyl-1,3,2-dioxaborolan-2-yl)-1H-pyrazole (Intermediate 1) using the general conditions employed in example 1 followed by removal of the Boc-protecting group using the conditions employed for the preparation of Intermediate 5.

### Intermediate 32

### Synthesis of benzyl 3-((5-bromo-1H-pyrrolo[2,3-c]pyridin-1-yl)methyl)pyrrolidine-1-carboxylate

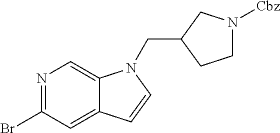 [SMILES: O=C(OCc1ccccc1)N1CCC(Cn2ccc3cc(Br)ncc32)C1]

Benzyl 3-((5-bromo-1H-pyrrolo[2,3-c]pyridin-1-yl)methyl)pyrrolidine-1-carboxylate can be prepared from 5-bromo-1H-pyrrolo[2,3-c]pyridine (intermediate 3) and benzyl 3-(tosyloxymethyl)pyrrolidine-1-carboxylate (intermediate 26) using the method described for the preparation of tert-butyl 4-((5-bromo-1H-pyrrolo[2,3-c]pyridin-1-yl)methyl)piperidine-1-carboxylate (intermediate 4).

### Intermediate 33

### Synthesis of 1-((1-benzyl-5-methylpyrrolidin-3-yl)methyl)-5-bromo-1H-pyrrolo[2,3-c]pyridine

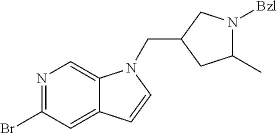

1-((1-benzyl-5-methylpyrrolidin-3-yl)methyl)-5-bromo-1H-pyrrolo[2,3-c]pyridine can be prepared from 5-bromo-1H-pyrrolo[2,3-c]pyridine (intermediate 3) and (1-benzyl-5-methylpyrrolidin-3-yl)methyl 4-methylbenzenesulfonate (intermediate 27) using the method described for the preparation of tert-butyl 4-((5-bromo-1H-pyrrolo[2,3-c]pyridin-1-yl)methyl)piperidine-1-carboxylate (intermediate 4).

### Intermediate 34

### Synthesis of 1-((1-benzyl-5,5-dimethylpyrrolidin-3-yl)methyl)-5-bromo-1H-pyrrolo [2,3-c]pyridine

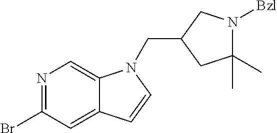

1-((1-Benzyl-5,5-dimethylpyrrolidin-3-yl)methyl)-5-bromo-1H-pyrrolo[2,3-c]pyridine can be prepared from 5-bromo-1H-pyrrolo[2,3-c]pyridine (intermediate 3) and (1-benzyl-5,5-dimethylpyrrolidin-3-yl)methyl 4-methylbenzenesulfonate (intermediate 30) using the method described for the preparation of tert-butyl 4-((5-bromo-1H-pyrrolo[2,3-c]pyridin-1-yl)methyl)piperidine-1-carboxylate (intermediate 4).

### Intermediate 35

### Synthesis of benzyl 4-((5-bromo-1H-pyrrolo[2,3-c]pyridin-1-yl)methyl)azepane-1-carboxylate

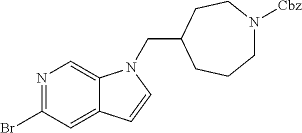 [SMILES: O=C(OCc1ccccc1)N1CCCC(Cn2ccc3cc(Br)ncc32)CC1]

Benzyl 4-((5-bromo-1H-pyrrolo[2,3-c]pyridin-1-yl)methyl)azepane-1-carboxylate can be prepared from 5-bromo-1H-pyrrolo[2,3-c]pyridine (intermediate 3) and benzyl 4-(tosyloxymethyl)azepane-1-carboxylate (intermediate 28) using the method described for the preparation of tert-butyl 4-((5-bromo-1H-pyrrolo[2,3-c]pyridin-1-yl)methyl)piperidine-1-carboxylate (intermediate 4).

### Intermediate 36

### Synthesis of rac.-benzyl 2-((5-bromo-1H-pyrrolo[2,3-c]pyridin-1-yl)methyl)-7-azabicyclo[2.2.1]heptane-7-carboxylate

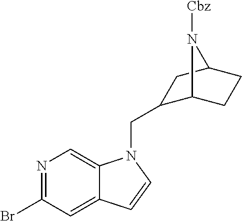 [SMILES: O=C(OCc1ccccc1)N1C2CCC1C(Cn1ccc3cc(Br)ncc31)C2]

Rac.-benzyl 2-((5-bromo-1H-pyrrolo[2,3-c]pyridin-1-yl)methyl)-7-azabicyclo[2.2.1]heptane-7-carboxylate can be prepared from 5-bromo-1H-pyrrolo[2,3-c]pyridine (intermediate 3) and benzyl 2-((tosyloxy)methyl)-7-azabicyclo[2.2.1]heptane-7-carboxylate (intermediate 29) using the method described for the preparation of tert-butyl 4-((5-bromo-1H-pyrrolo[2,3-c]pyridin-1-yl)methyl)piperidine-1-carboxylate (intermediate 4).

### Intermediates 37 and 38

### Synthesis of cis-(1-benzyl-5-methylpyrrolidin-3-yl) methyl 4-methylbenzenesulfonate and trans-(1-benzyl-5-methylpyrrolidin-3-yl) methyl 4-methylbenzenesulfonate

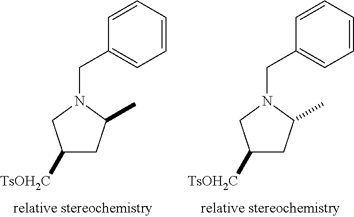

Step 1. Synthesis of 1-benzyl-5-methylpyrrolidin-2-one.

To a stirred solution of 5-methylpyrrolidin-2-one (200 g, 2.02 mol, 1.0 eq) in DMF (1.5 L), sodium hydride (60% suspension on mineral oil, 131 g, 3.3 mol, 1.5 eq) was slowly added at 0° C., followed by benzyl bromide (292 mL, 2.42 mol, 1.2 eq) and the mixture was allowed stir at room temperature for 3 hr. After completion of the reaction (monitored by TLC, 20% ethyl acetate-hexane, KMnO₄, R_{f}=0.45), the reaction was quenched by adding ice cubes and the mixture was extracted with ethyl acetate (500 mL). The organic extract was dried over anhydrous sodium sulfate, filtered and concentrated under reduced pressure. The crude product was purified by column chromatography on silica gel (100-200 mesh), eluting with 10% ethyl acetate in hexanes to afford 1-benzyl-5-methylpyrrolidin-2-one in two fractions. The first fraction contained 200 g of 1-benzyl-5-methylpyrrolidin-2-one (yield 52.4%, LC-MS: purity: 95%) and the second fraction contained an additional 100 g (yield 26.2%, LC-MS: purity: 83%) as an oily liquid. (ES⁺): m/z 190.1 (M+H⁺); tr=1.21, 1.61 min.

Step 2: Synthesis of methyl 1-benzyl-5-methyl-2-oxopyrrolidine-3-carboxylate and 1-benzyl-5-methyl-2-oxopyrrolidine-3-carboxylic acid.

n-BuLi (2.5M in hexanes, 215 mL, 0.528 mol, 2.0 eq) was slowly added to a stirred solution of diisopropyl amine (78.4 mL, 0.555 mol, 2.1 eq) in THF (500 mL), at −78° C. and stirring was continued for 40 min, during which time, the temperature of the reaction was allowed to rise to −20° C. The mixture was again cooled to −78° C., a solution of 1-benzyl-5-methylpyrrolidin-2-one (50 g, 0.265 mol, 1.0 eq) in THF (5.0 L) was added and stirring was continued for 45 min, maintaining the same temperature. Then dimethyl carbonate (44.5 mL, 0.528 mol, 2.0 eq) was added to the above mixture and stirring continued for 5 h, during which time, the temperature of the reaction mixture was allowed to rise to room temperature. After completion of the reaction (monitored by TLC, 30% ethyl acetate-hexanes, KMnO₄, RU 0.65), the reaction was quenched by slowly adding 1M aq NH₄Cl at 0° C. and the mixture was extracted with ethyl acetate (3 L). The organic extract was dried over anhydrous sodium sulfate, filtered and concentrated under reduced pressure to afford crude methyl 1-benzyl-5-methyl-2-oxopyrrolidine-3-carboxylate (4.5 g, 6.8% mixture of diastereomers) as a brown sticky mass. LC-MS purity: 37.5%, (ES⁺) m/z: 248.1 (M+H⁺), tr=1.32, 1.71.

The aqueous extract was acidified with 2N HCl to pH 2 and again extracted with ethyl acetate (5 L). The organic extract was dried over anhydrous sodium sulfate, filtered and concentrated under reduced pressure to afford 1-benzyl-5-methyl-2-oxopyrrolidine-3-carboxylic acid (40.1 g, 65.2%, mixture of diastereomers) brown sticky mass. LC-MS purity: 68.9% (ES⁺): m/z 234.1 (M+H⁺). tr=1.46, 1.49.

Step 3: Synthesis of (1-benzyl-5-methylpyrrolidin-3-yl)methanol.

Lithium aluminium hydride (2M in THF, 253 mL, 252 mmol, 2.35 eq) was added to a stirred solution of 1-benzyl-5-methyl-2-oxopyrrolidine-3-carboxylic acid (25 g, 107 mmol, 1.0 eq) in THF (250 mL), at 0° C. and stirring was continued for 3 h, during which time temperature of the reaction was allowed to rise to room temperature. After completion (monitored by TLC, 30% ethyl acetate-hexanes, KMnO₄, R_{f}=0.65), the reaction was again cooled to 0° C. and excess lithium aluminium hydride was quenched by addition of 15 mL of water very slowly over a period of 3 hr. The white precipitate formed was filtered through a celite bed and the filtrate was concentrated under reduced pressure to afford (1-benzyl-5-methylpyrrolidin-3-yl)methanol (9 g, mixture of diastereomers) as a brown sticky mass, which was used as such in the next step. LC-MS purity: 68.03%. (ES⁺): m/z 206.1 (M+H⁺).

Step 4: Synthesis of (1-benzyl-5-methylpyrrolidin-3-yl)methyl 4-methylbenzenesulfonate.

To a stirred solution of (1-benzyl-5-methylpyrrolidin-3-yl)methanol (40 g, 195 mmol, 1.0 eq) in dichloromethane (250 mL), triethylamine (81.5 mL, 59 mmol, 3.0 eq) was slowly added at 0° C. followed by tosyl chloride (44.6 g, 234 mmol, 1.2 eq) and the mixture was allowed to stir at room temperature for 12 hr. After completion of the reaction (monitored by TLC, 50% ethyl acetate-hexanes, R_{f}=0.65), saturated aqueous NaHCO₃ solution (25 mL) was added and the organic layer was separated. The aqueous layer was further extracted with dichloromethane (120 mL). The combined organic extract was dried over anhydrous sodium sulfate, filtered and concentrated under reduced pressure to afford (1-benzyl-5-methylpyrrolidin-3-yl)methyl 4-methylbenzenesulfonate (32 g, mixture of diastereomers) as brown sticky mass. LC-MS purity: 94.65%. (ES⁺): m/z 360.16 (M+H⁺). tr=1.40, 1.53.

Step 5: Separation of cis and trans isomers of (1-benzyl-5-methylpyrrolidin-3-yl)methyl 4-methylbenzenesulfonate.

A mixture of the cis and trans isomers of (1-benzyl-5-methylpyrrolidin-3-yl)methyl 4-methylbenzenesulfonate (63 g) was purified by column chromatography on silica gel, (5 kg) 100-200 mesh, eluting with 10% ethyl acetate in hexanes to obtain cis-(1-benzyl-5-methylpyrrolidin-3-yl) methyl 4-methylbenzenesulfonate (19.6 g, 31.1%) as a pale brown liquid and trans-(1-benzyl-5-methylpyrrolidin-3-yl) methyl 4-methylbenzenesulfonate (25.9 g, 41.1) as an off white solid.

Data for cis-(1-benzyl-5-methylpyrrolidin-3-yl) methyl 4-methylbenzenesulfonate (Intermediate 37)

LC-MS purity: 91.78%; (ES⁺): m/z 360.32 (M+H⁺); tr=4.42 min.

¹H NMR (400 MHz, CDCl₃) δ 7.73 (d, J 8.4 Hz, 2H), 7.31-7.22 (m, 7H), 3.95 (d, J 8.4 Hz, 1H), 3.87 (dd, J=1.6, 8.4 Hz, 2H), 3.06 (d, J 13.2 Hz, 1H), 2.62 (dd, J=2.4, 10.4 Hz, 1H), 2.44 (s, 3H), 2.42-2.00 (m, 4H), 1.09-1.00 (m, 4H).

Data for trans-(1-benzyl-5-methylpyrrolidin-3-yl)methyl 4-methylbenzenesulfonate (Intermediate 38):

LC-MS purity: 94.64%; (ES⁺): m/z 360.32 (M+H⁺); tr=4.64 min.

¹H NMR (400 MHz, CDCl₃) δ 7.73 (d, J=8.0 Hz, 2H), 7.33-7.21 (m, 7H), 3.98 (m, 3H), 3.04 (d, J 12.8 Hz, 1H), 2.95 (dd, J=2.0, 7.2 Hz, 1H), 2.45 (s, 3H), 2.42-2.37 (m, 2H), 1.79 (d, J=8.4 Hz, 1H), 1.66-1.54 (m, 2H), 1.12 (d, 3H).

### Intermediate 39

### Synthesis of rac.-cis-1-((1-benzyl-5-methylpyrrolidin-3-yl)methyl)-5-bromo-1H-pyrrolo [2,3-c]pyridine

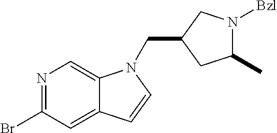

Rac.-cis-1-((1-benzyl-5-methylpyrrolidin-3-yl)methyl)-5-bromo-1H-pyrrolo[2,3-c]pyridine can be prepared from 5-bromo-1H-pyrrolo[2,3-c]pyridine (intermediate 3) and cis-(1-benzyl-5-methylpyrrolidin-3-yl) methyl 4-methylbenzenesulfonate (Intermediate 37) using the method described for the preparation of tert-butyl 4-((5-bromo-1H-pyrrolo[2,3-c]pyridin-1-yl)methyl)piperidine-1-carboxylate (intermediate 4).

### Intermediate 40

### Synthesis of rac.-trans-1-((1-benzyl-5-methylpyrrolidin-3-yl)methyl)-5-bromo-1H-pyrrolo [2,3-c]pyridine

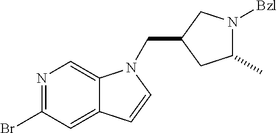

Rac.-trans-1-((1-benzyl-5-methylpyrrolidin-3-yl)methyl)-5-bromo-1H-pyrrolo[2,3-c]pyridine can be prepared from 5-bromo-1H-pyrrolo[2,3-c]pyridine (intermediate 3) and trans-(1-benzyl-5-methylpyrrolidin-3-yl) methyl 4-methylbenzenesulfonate (Intermediate 38) using the method described for the preparation of tert-butyl 4-((5-bromo-1H-pyrrolo[2,3-c]pyridin-1-yl)methyl)piperidine-1-carboxylate (intermediate 4).

### Intermediate 41

### Synthesis of 1-(pyrrolidin-3-ylmethyl)-5-(1-(tetrahydro-2H-pyran-2-yl)-1H-pyrazol-4-yl)-1H-pyrrolo [2,3-c]pyridine

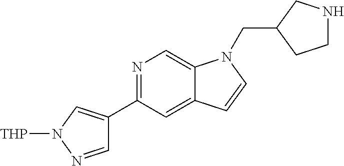

1-(Pyrrolidin-3-ylmethyl)-5-(1-(tetrahydro-2H-pyran-2-yl)-1H-pyrazol-4-yl)-1H-pyrrolo[2,3-c]pyridine can be prepared by coupling benzyl 3-((5-bromo-1H-pyrrolo[2,3-c]pyridin-1-yl)methyl)pyrrolidine-1-carboxylate (Intermediate 32) and 1-(tetrahydro-2H-pyran-2-yl)-4-(4,4,5,5-tetramethyl-1,3,2-dioxaborolan-2-yl)-1H-pyrazole (intermediate 1) using the coupling method described for the preparation of (4-((5-(1H-pyrazol-4-yl)-1H-pyrrolo[2,3-c]pyridin-1-yl)methyl)piperidin-1-yl)(phenyl)methanone (example 1) followed by deprotection by hydrogenating an ethanolic solution of the intermediate benzyoxycarbonyl derivative over 10% Pd on charcoal (50% moisture) in a Parr shaker at 40 psi H₂.

### Intermediate 42

### Synthesis of 1-((5-methylpyrrolidin-3-yl)methyl)-5-(1-(tetrahydro-2H-pyran-2-yl)-1H-pyrazol-4-yl)-1H-pyrrolo [2,3-c]pyridine

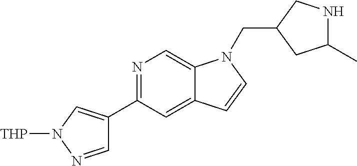

1-((5-Methylpyrrolidin-3-yl)methyl)-5-(1-(tetrahydro-2H-pyran-2-yl)-1H-pyrazol-4-yl)-1H-pyrrolo[2,3-c]pyridine can be prepared by coupling 1-((1-benzyl-5-methylpyrrolidin-3-yl)methyl)-5-bromo-1H-pyrrolo[2,3-c]pyridine (Intermediate 33) and 1-(tetrahydro-2H-pyran-2-yl)-4-(4,4,5,5-tetramethyl-1,3,2-dioxaborolan-2-yl)-1H-pyrazole (intermediate 1) using the coupling method described for the preparation of (4-((5-(1H-pyrazol-4-yl)-1H-pyrrolo[2,3-c]pyridin-1-yl)methyl)piperidin-1-yl)(phenyl)methanone (example 1) followed by deprotection by treating an aqueous methanolic solution of the intermediate benzyl derivative with Pd(OH)₂ on carbon (20% w/w, 50% moisture) followed by ammonium formate at room temperature and then a 3 hr reflux.

### Intermediate 43

### Synthesis of 1-((5,5-dimethylpyrrolidin-3-yl)methyl)-5-(1-(tetrahydro-2H-pyran-2-yl)-1H-pyrazol-4-yl)-1H-pyrrolo [2,3-c]pyridine

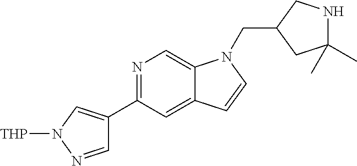

1-((5,5-Dimethylpyrrolidin-3-yl)methyl)-5-(1-(tetrahydro-2H-pyran-2-yl)-1H-pyrazol-4-yl)-1H-pyrrolo[2,3-c]pyridine can be prepared by coupling 1-((1-benzyl-5,5-dimethylpyrrolidin-3-yl)methyl)-5-bromo-1H-pyrrolo[2,3-c]pyridine (Intermediate 34) and 1-(tetrahydro-2H-pyran-2-yl)-4-(4,4,5,5-tetramethyl-1,3,2-dioxaborolan-2-yl)-1H-pyrazole (Intermediate 1) using the coupling method described for the preparation of (4-((5-(1H-pyrazol-4-yl)-1H-pyrrolo[2,3-c]pyridin-1-yl)methyl)piperidin-1-yl)(phenyl)methanone (Example 1) followed by deprotection by treating an aqueous methanolic solution of the intermediate benzyl derivative with Pd(OH)₂ on carbon (20% w/w, 50% moisture) followed by ammonium formate at room temperature and then heating to reflux.

### Intermediate 44

### Synthesis of rac.-cis-1-((5-methylpyrrolidin-3-yl)methyl)-5-(1-(tetrahydro-2H-pyran-2-yl)-1H-pyrazol-4-yl)-1H-pyrrolo [2,3-c]pyridine

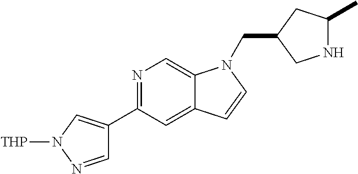

Rac.-cis-1-((5-methylpyrrolidin-3-yl)methyl)-5-(1-(tetrahydro-2H-pyran-2-yl)-1H-pyrazol-4-yl)-1H-pyrrolo[2,3-c]pyridine can be prepared by coupling rac.-cis-1-((1-benzyl-5-methylpyrrolidin-3-yl)methyl)-5-bromo-1H-pyrrolo[2,3-c]pyridine (Intermediate 39) and 1-(tetrahydro-2H-pyran-2-yl)-4-(4,4,5,5-tetramethyl-1,3,2-dioxaborolan-2-yl)-1H-pyrazole (intermediate 1) using the coupling method described for the preparation of (4-((5-(1H-pyrazol-4-yl)-1H-pyrrolo[2,3-c]pyridin-1-yl)methyl)piperidin-1-yl)(phenyl)methanone (example 1) followed by deprotection of the benzyl derivative by treatment with Pd(OH)₂ on carbon (20% w/w, 50% moisture) followed by ammonium formate at room temperature and then heating to reflux.

### Intermediate 45

### Synthesis of rac.-trans-1-((5-methylpyrrolidin-3-yl)methyl)-5-(1-(tetrahydro-2H-pyran-2-yl)-1H-pyrazol-4-yl)-1H-pyrrolo [2,3-c]pyridine

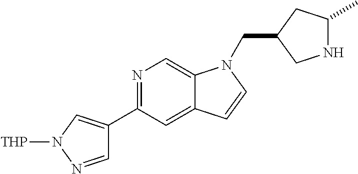

Rac.-trans-1-((5-methylpyrrolidin-3-yl)methyl)-5-(1-(tetrahydro-2H-pyran-2-yl)-1H-pyrazol-4-yl)-1H-pyrrolo[2,3-c]pyridine can be prepared by coupling rac.-trans-1-((1-benzyl-5-methylpyrrolidin-3-yl)methyl)-5-bromo-1H-pyrrolo[2,3-c]pyridine (Intermediate 40) and 1-(tetrahydro-2H-pyran-2-yl)-4-(4,4,5,5-tetramethyl-1,3,2-dioxaborolan-2-yl)-1H-pyrazole (intermediate 1) using the coupling method described for the preparation of (4-((5-(1H-pyrazol-4-yl)-1H-pyrrolo[2,3-c]pyridin-1-yl)methyl)piperidin-1-yl)(phenyl)methanone (example 1) followed by deprotection of the benzyl derivative with Pd(OH)₂ on carbon (20% w/w, 50% moisture) followed by ammonium formate at room temperature and then heating to reflux.

### Intermediate 46

### Synthesis of 1-(azepan-4-ylmethyl)-5-(1-(tetrahydro-2H-pyran-2-yl)-1H-pyrazol-4-yl)-1H-pyrrolo[2,3-c]pyridine

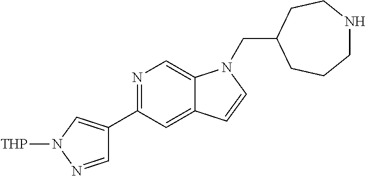

1-(Azepan-4-ylmethyl)-5-(1-(tetrahydro-2H-pyran-2-yl)-1H-pyrazol-4-yl)-1H-pyrrolo[2,3-c]pyridine can be prepared by coupling benzyl 4-((5-bromo-1H-pyrrolo[2,3-c]pyridin-1-yl)methyl)azepane-1-carboxylate (Intermediate 35) and 1-(tetrahydro-2H-pyran-2-yl)-4-(4,4,5,5-tetramethyl-1,3,2-dioxaborolan-2-yl)-1H-pyrazole (intermediate 1) using the coupling method described for the preparation of (4-((5-(1H-pyrazol-4-yl)-1H-pyrrolo[2,3-c]pyridin-1-yl)methyl)piperidin-1-yl)(phenyl)methanone (example 1) followed by deprotection of the intermediate benzyoxycarbonyl derivative over 10% Pd on charcoal (50% moisture) in a Parr shaker at 40 psi H₂.

### Intermediate 47

### Synthesis of rac.-1-((-7-azabicyclo[2.2.1]heptan-2-yl)methyl)-5-(1-(tetrahydro-2H-pyran-2-yl)-1H-pyrazol-4-yl)-1H-pyrrolo [2,3-c]pyridine

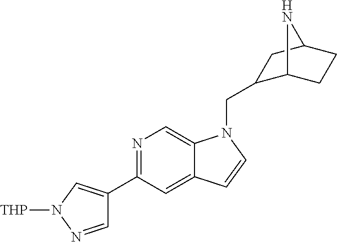

1-((-7-Azabicyclo[2.2.1]heptan-2-yl)methyl)-5-(1-(tetrahydro-2H-pyran-2-yl)-1H-pyrazol-4-yl)-1H-pyrrolo[2,3-c]pyridine can be prepared by coupling benzyl 4-((5-bromo-1H-pyrrolo[2,3-c]pyridin-1-yl)methyl)azepane-1-carboxylate (Intermediate 36) and 1-(tetrahydro-2H-pyran-2-yl)-4-(4,4,5,5-tetramethyl-1,3,2-dioxaborolan-2-yl)-1H-pyrazole (intermediate 1) using the coupling method described for the preparation of (4-((5-(1H-pyrazol-4-yl)-1H-pyrrolo[2,3-c]pyridin-1-yl)methyl)piperidin-1-yl)(phenyl)methanone (example 1) followed by deprotection of the intermediate benzyoxycarbonyl derivative over 10% Pd on charcoal (50% moisture) in a Parr shaker at 40 psi H₂.

### Intermediate 48

### Synthesis of of 1-(piperidin-4-ylmethyl)-5-(1-(tetrahydro-2H-pyran-2-yl)-1H-pyrazol-4-yl)-1H-pyrrolo [2,3-b]pyridine

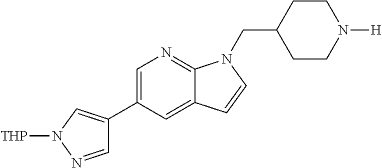

1-(Piperidin-4-ylmethyl)-5-(1-(tetrahydro-2H-pyran-2-yl)-1H-pyrazol-4-yl)-1H-pyrrolo[2,3-b]pyridine can be prepared in two steps from tert-butyl 4-((5-bromo-1H-pyrrolo[2,3-b]pyridin-1-yl)methyl)piperidine-1-carboxylate (Intermediate 14) by palladium catalyzed coupling with 1-(tetrahydro-2H-pyran-2-yl)-4-(4,4, 5,5-tetramethyl-1,3,2-dioxaborolan-2-yl)-1H-pyrazole (Intermediate 1) using the general conditions employed in example 9 followed by removal of the Boc-protecting group using the conditions employed for the preparation of Intermediate 15.

### Intermediate 49

### Synthesis of benzyl 3-((5-bromo-1H-pyrrolo[2,3-b]pyridin-1-yl)methyl)pyrrolidine-1-carboxylate

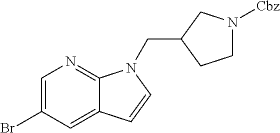 [SMILES: O=C(OCc1ccccc1)N1CCC(Cn2ccc3cc(Br)cnc32)C1]

Benzyl 3-((5-bromo-1H-pyrrolo[2,3-b]pyridin-1-yl)methyl)pyrrolidine-1-carboxylate can be prepared from 5-bromo-1H-pyrrolo[2,3-b]pyridine and benzyl 3-(tosyloxymethyl)pyrrolidine-1-carboxylate (Intermediate 26) using the method described for the preparation of tert-butyl 4-((5-bromo-1H-pyrrolo[2,3-b]pyridin-1-yl)methyl)piperidine-1-carboxylate (intermediate 14).

### Intermediate 50

### Synthesis of 1-((1-benzyl-5-methylpyrrolidin-3-yl)methyl)-5-bromo-1H-pyrrolo[2,3-b]pyridine

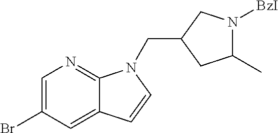

1-((1-benzyl-5-methylpyrrolidin-3-yl)methyl)-5-bromo-1H-pyrrolo[2,3-b]pyridine can be prepared from 5-bromo-1H-pyrrolo[2,3-b]pyridine and (1-benzyl-5-methylpyrrolidin-3-yl)methyl 4-methylbenzenesulfonate (intermediate 27) using the method described for the preparation of tert-butyl 4-((5-bromo-1H-pyrrolo[2,3-b]pyridin-1-yl)methyl)piperidine-1-carboxylate (intermediate 14).

### Intermediate 51

### Synthesis of 1-((1-benzyl-5,5-dimethylpyrrolidin-3-yl)methyl)-5-bromo-1H-pyrrolo[2,3-b]pyridine

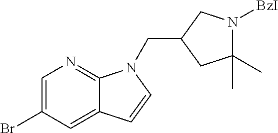

1-((1-Benzyl-5,5-dimethylpyrrolidin-3-yl)methyl)-5-bromo-1H-pyrrolo[2,3-b]pyridine can be prepared from 5-bromo-1H-pyrrolo[2,3-b]pyridine and (1-benzyl-5,5-dimethylpyrrolidin-3-yl)methyl 4-methylbenzenesulfonate (intermediate 30) using the method described for the preparation of tert-butyl 4-((5-bromo-1H-pyrrolo[2,3-b]pyridin-1-yl)methyl)piperidine-1-carboxylate (intermediate 14).

### Intermediate 52

### Synthesis of benzyl 4-((5-bromo-1H-pyrrolo[2,3-b]pyridin-1-yl)methyl)azepane-1-carboxylate

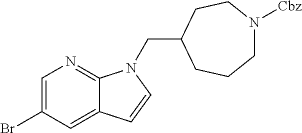 [SMILES: O=C(OCc1ccccc1)N1CCCC(Cn2ccc3cc(Br)cnc32)CC1]

Benzyl 4-((5-bromo-1H-pyrrolo[2,3-b]pyridin-1-yl)methyl)azepane-1-carboxylate can be prepared from 5-bromo-1H-pyrrolo[2,3-b]pyridine and benzyl 4-(tosyloxymethyl)azepane-1-carboxylate (intermediate 28) using the method described for the preparation of tert-butyl 4-((5-bromo-1H-pyrrolo[23-b]pyridin-1-yl)methyl)piperidine-1-carboxylate (intermediate 14).

### Intermediate 53

### Synthesis of rac.-benzyl 2-((5-bromo-1H-pyrrolo[2,3-c]pyridin-1-yl)methyl)-7-azabicyclo[2.2.1]heptane-7-carboxylate

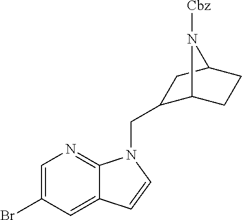 [SMILES: O=C(OCc1ccccc1)N1C2CCC1C(Cn1ccc3cc(Br)cnc31)C2]

Rac.-benzyl 2-((5-bromo-1H-pyrrolo[3,2-b]pyridin-1-yl)methyl)-7-azabicyclo[2.2.1]heptane-7-carboxylate can be prepared from 5-bromo-1H-pyrrolo[2,3-b]pyridine and benzyl 2-((tosyloxy)methyl)-7-azabicyclo[2.2.1]heptane-7-carboxylate (intermediate 29) using the method described for the preparation of tert-butyl 4-((5-bromo-1H-pyrrolo[2,3-b]pyridin-1-yl)methyl)piperidine-1-carboxylate (intermediate 14).

### Intermediate 54

### Synthesis of rac-cis-1-((1-benzyl-5-methylpyrrolidin-3-yl)methyl)-5-bromo-1H-pyrrolo[2,3-b]pyridine

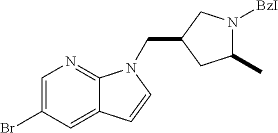

Rac-cis-1-((1-benzyl-5-methylpyrrolidin-3-yl)methyl)-5-bromo-1H-pyrrolo[2,3-b]pyridine can be prepared from 5-bromo-1H-pyrrolo[2,3-b]pyridine and rac-cis-(1-benzyl-5-methylpyrrolidin-3-yl) methyl 4-methylbenzenesulfonate (Intermediate 37) using the method described for the preparation of tert-butyl 4-((5-bromo-1H-pyrrolo[2,3-b]pyridin-1-yl)methyl)piperidine-1-carboxylate (intermediate 14).

### Intermediate 55

### Synthesis of rac-trans-1-((1-benzyl-5-methylpyrrolidin-3-yl)methyl)-5-bromo-1H-pyrrolo[2,3-b]pyridine

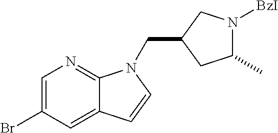

Rac-trans-1-((1-benzyl-5-methylpyrrolidin-3-yl)methyl)-5-bromo-1H-pyrrolo[2,3-b]pyridine can be prepared from 5-bromo-1H-pyrrolo[2,3-b]pyridine and rac-trans-(1-benzyl-5-methylpyrrolidin-3-yl) methyl 4-methylbenzenesulfonate (Intermediate 38) using the method described for the preparation of tert-butyl 4-((5-bromo-1H-pyrrolo[2,3-b]pyridin-1-yl)methyl)piperidine-1-carboxylate (intermediate 14).

### Intermediate 56

### Synthesis of 1-(pyrrolidin-3-ylmethyl)-5-(1-(tetrahydro-2H-pyran-2-yl)-1H-pyrazol-4-yl)-1H-pyrrolo [2,3-b]pyridine

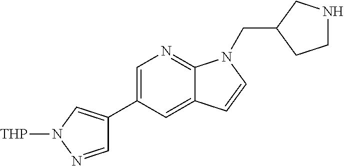

1-(Pyrrolidin-3-ylmethyl)-5-(1-(tetrahydro-2H-pyran-2-yl)-1H-pyrazol-4-yl)-1H-pyrrolo[2,3-b]pyridine can be prepared by coupling benzyl 3-((5-bromo-1H-pyrrolo[2,3-b]pyridin-1-yl)methyl)pyrrolidine-1-carboxylate (Intermediate 49) and 1-(tetrahydro-2H-pyran-2-yl)-4-(4,4,5,5-tetramethyl-1,3,2-dioxaborolan-2-yl)-1H-pyrazole (intermediate 1) using the coupling method described for the preparation of (4-((5-(1H-pyrazol-4-yl)-1H-pyrrolo[2,3-b]pyridin-1-yl)methyl)piperidin-1-yl)(phenyl)methanone (example 9) followed by deprotection by hydrogenating an ethanolic solution of the intermediate benzyoxycarbonyl derivative over 10% Pd on charcoal (50% moisture) in a Parr shaker at 40 psi H₂.

### Intermediate 57

### Synthesis of 1-((5-methylpyrrolidin-3-yl)methyl)-5-(1-(tetrahydro-2H-pyran-2-yl)-1H-pyrazol-4-yl)-1H-pyrrolo [2,3-b]pyridine

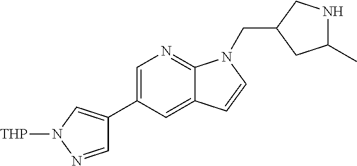

1-((5-Methylpyrrolidin-3-yl)methyl)-5-(1-(tetrahydro-2H-pyran-2-yl)-1H-pyrazol-4-yl)-1H-pyrrolo[2,3-b]pyridine can be prepared by coupling 1-((1-benzyl-5-methylpyrrolidin-3-yl)methyl)-5-bromo-1H-pyrrolo[2,3-b]pyridine (Intermediate 50) and 1-(tetrahydro-2H-pyran-2-yl)-4-(4,4,5,5-tetramethyl-1,3,2-dioxaborolan-2-yl)-1H-pyrazole (intermediate 1) using the coupling method described for the preparation of (4-((5-(1H-pyrazol-4-yl)-1H-pyrrolo[2,3-b]pyridin-1-yl)methyl)piperidin-1-yl)(phenyl)methanone (example 1) followed by deprotection by treating an aqueous methanolic solution of the intermediate benzyl derivative with Pd(OH)₂ on carbon (20% w/w, 50% moisture) followed by ammonium formate at room temperature followed by reflux.

### Intermediate 58

### Synthesis of rac.-1-((5,5-dimethylpyrrolidin-3-yl)methyl)-5-(1-(tetrahydro-2H-pyran-2-yl)-1H-pyrazol-4-yl)-1H-pyrrolo [2,3-b]pyridine

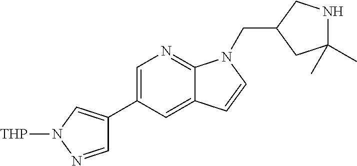

1-((5,5-dimethylpyrrolidin-3-yl)methyl)-5-(1-(tetrahydro-2H-pyran-2-yl)-1H-pyrazol-4-yl)-1H-pyrrolo[2,3-b]pyridine can be prepared by coupling 1-((1-benzyl-5,5-dimethylpyrrolidin-3-yl)methyl)-5-bromo-1H-pyrrolo[2,3-b]pyridine (Intermediate 51) and 1-(tetrahydro-2H-pyran-2-yl)-4-(4,4,5,5-tetramethyl-1,3,2-dioxaborolan-2-yl)-1H-pyrazole (Intermediate 1) using the coupling method described for the preparation of (4-((5-(1H-pyrazol-4-yl)-1H-pyrrolo[2,3-b]pyridin-1-yl)methyl)piperidin-1-yl)(phenyl)methanone (Example 1) followed by deprotection by treating an aqueous methanolic solution of the intermediate benzyl derivative with Pd(OH)₂ on carbon (20% w/w, 50% moisture) followed by ammonium formate at room temperature and then heating to reflux.

### Intermediate 59

### Synthesis of Rac.-cis-1-((5-methylpyrrolidin-3-yl)methyl)-5-(1-(tetrahydro-2H-pyran-2-yl)-1H-pyrazol-4-yl)-1H-pyrrolo [2,3-b]pyridine

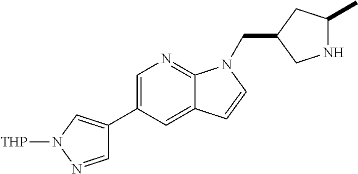

Rac.-cis-1-((5-methylpyrrolidin-3-yl)methyl)-5-(1-(tetrahydro-2H-pyran-2-yl)-1H-pyrazol-4-yl)-1H-pyrrolo[2,3-c]pyridine can be prepared by coupling rac-cis-1-((1-benzyl-5-methylpyrrolidin-3-yl)methyl)-5-bromo-1H-pyrrolo[2,3-b]pyridine (Intermediate 54) and 1-(tetrahydro-2H-pyran-2-yl)-4-(4,4,5,5-tetramethyl-1,3,2-dioxaborolan-2-yl)-1H-pyrazole (intermediate 1) using the coupling method described for the preparation of (4-((5-(1H-pyrazol-4-yl)-1H-pyrrolo[2,3-b]pyridin-1-yl)methyl)piperidin-1-yl)(phenyl)methanone (example 1) followed by deprotection of the benzyl derivative by treatment with Pd(OH)₂ on carbon (20% w/w, 50% moisture) followed by ammonium formate at room temperature and then heating to reflux.

### Intermediate 60

### Synthesis of Rac.-trans-1-((5-methylpyrrolidin-3-yl)methyl)-5-(1-(tetrahydro-2H-pyran-2-yl)-1H-pyrazol-4-yl)-1H-pyrrolo [2,3-b]pyridine

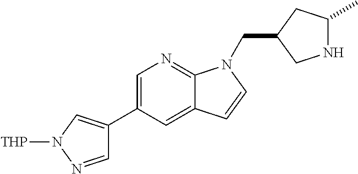

Rac.-trans-1-((5-methylpyrrolidin-3-yl)methyl)-5-(1-(tetrahydro-2H-pyran-2-yl)-1H-pyrazol-4-yl)-1H-pyrrolo[2,3-b]pyridine can be prepared by coupling trans-1-((1-benzyl-5-methylpyrrolidin-3-yl)methyl)-5-bromo-1H-pyrrolo[2,3-b]pyridine (Intermediate 55) and 1-(tetrahydro-2H-pyran-2-yl)-4-(4,4,5,5-tetramethyl-1,3,2-dioxaborolan-2-yl)-1H-pyrazole (intermediate 1) using the coupling method described for the preparation of (4-((5-(1H-pyrazol-4-yl)-1H-pyrrolo[2,3-c]pyridin-1-yl)methyl)piperidin-1-yl)(phenyl)methanone (example 1) followed by deprotection of the benzyl derivative with Pd(OH)₂ on carbon (20% w/w, 50% moisture) followed by ammonium formate at room temperature and then heating to reflux.

### Intermediate 61

### Synthesis of 1-(azepan-4-ylmethyl)-5-(1-(tetrahydro-2H-pyran-2-yl)-1H-pyrazol-4-yl)-1H-pyrrolo[2,3-b]pyridine

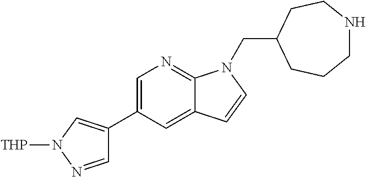

1-(Azepan-4-ylmethyl)-5-(1-(tetrahydro-2H-pyran-2-yl)-1H-pyrazol-4-yl)-1H-pyrrolo[2,3-b]pyridine can be prepared by coupling benzyl 4-((5-bromo-1H-pyrrolo[2,3-b]pyridin-1-yl)methyl)azepane-1-carboxylate (Intermediate 52) and 1-(tetrahydro-2H-pyran-2-yl)-4-(4,4,5,5-tetramethyl-1,3,2-dioxaborolan-2-yl)-1H-pyrazole (intermediate 1) using the coupling method described for the preparation of (4-((5-(1H-pyrazol-4-yl)-1H-pyrrolo[2,3-b]pyridin-1-yl)methyl)piperidin-1-yl)(phenyl)methanone (example 9) followed by deprotection of the intermediate benzyoxycarbonyl derivative over 10% Pd on charcoal (50% moisture) in a Parr shaker at 40 psi H₂.

### Intermediate 62

### Synthesis of Rac.-benzyl 2-((5-(1-(tetrahydro-2H-pyran-2-yl)-1H-pyrazol-4-yl)-1H-pyrrolo [2,3-b]pyridin-1-yl)methyl)-7-azabicyclo [2.2.1]heptane-7-carboxylate

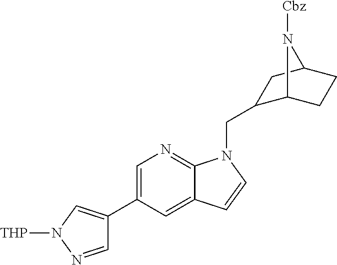

Rac.-benzyl 2-((5-(1-(tetrahydro-2H-pyran-2-yl)-1H-pyrazol-4-yl)-1H-pyrrolo[2,3-b]pyridin-1-yl)methyl)-7-azabicyclo[2.2.1]heptane-7-carboxylate can be prepared by coupling rac-benzyl 2-((5-bromo-1H-pyrrolo[3,2-b]pyridin-1-yl)methyl)-7-azabicyclo[2.2.1]heptane-7-carboxylate (Intermediate 53) and 1-(tetrahydro-2H-pyran-2-yl)-4-(4,4,5,5-tetramethyl-1,3,2-dioxaborolan-2-yl)-1H-pyrazole (intermediate 1) using the coupling method described for the preparation of (4-((5-(1H-pyrazol-4-yl)-1H-pyrrolo[2,3-b]pyridin-1-yl)methyl)piperidin-1-yl)(phenyl)methanone (example 9) followed by deprotection of the intermediate benzyoxycarbonyl derivative over 10% Pd on charcoal (50% moisture) in a Parr shaker at 40 psi H₂.

### Intermediate 63

### Synthesis of 3-phenyl-1-(4-((5-(4,4,5,5-tetramethyl-1,3,2-dioxaborolan-2-yl)-1H-pyrrolo[2,3-c]pyridin-1-yl)methyl)piperidin-1-yl)propan-1-one

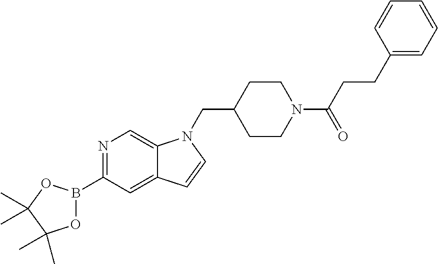 [SMILES: CC1(C)OB(c2cc3ccn(CC4CCN(C(=O)CCc5ccccc5)CC4)c3cn2)OC1(C)C]

3-Phenyl-1-(4-((5-(4,4,5,5-tetramethyl-1,3,2-dioxaborolan-2-yl)-1H-pyrrolo[2,3-c]pyridin-1-yl)methyl)piperidin-1-yl)propan-1-one can be prepared by reaction of bis(pinacolato)diboron (1.5 eq) with a solution of 1-(4-((5-bromo-1H-pyrrolo[2,3-c]pyridin-1-yl)methyl)piperidin-1-yl)-3-phenylpropan-1-one (Intermediate 7) (1.0 eq) in 1,4-dioxane 80° C. in the presence of potassium acetate (3 eq) under an argon atmosphere and employing PdCl₂(dppf)-dichloromethane (0.05 eq) as a catalyst for approximately 5 hr.

### Intermediate 64

### Synthesis of 3-phenyl-1-(4-((5-(4,4,5,5-tetramethyl-1,3,2-dioxaborolan-2-yl)-1H-pyrrolo[2,3-b]pyridin-1-yl)methyl)piperidin-1-yl)propan-1-one

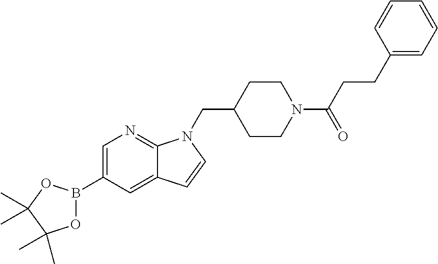 [SMILES: CC1(C)OB(c2cnc3c(ccn3CC3CCN(C(=O)CCc4ccccc4)CC3)c2)OC1(C)C]

(2′. 3-Phenyl-1-(4-((5-(4,4,5,5-tetramethyl-1,3,2-dioxaborolan-2-yl)-1H-pyrrolo[2,3-b]pyridin-1-yl)methyl)piperidin-1-yl)propan-1-one can be prepared by reaction of bis(pinacolato)diboron (1.5 eq) with a solution of 1-(4-((5-bromo-1H-pyrrolo[2,3-b]pyridin-1-yl)methyl)piperidin-1-yl)-3-phenylpropan-1-one (Intermediate 17) (1.0 eq) in 1,4-dioxane 80° C. in the presence of potassium acetate (3 eq) under an argon atmosphere and employing PdCl₂(dppf)-dichloromethane (0.05 eq) as a catalyst for approximately 5 hr.

### Example 1

### Synthesis of (4-((5-(1H-pyrazol-4-yl)-1H-pyrrolo[2,3-c]pyridin-1-yl)methyl)piperidin-1-yl)(phenyl)methanone

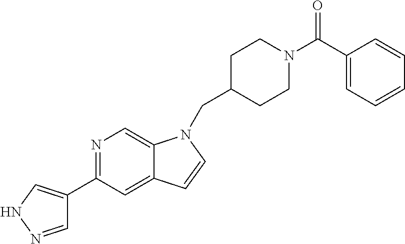 [SMILES: O=C(c1ccccc1)N1CCC(Cn2ccc3cc(-c4cn[nH]c4)ncc32)CC1]

Commercially available 4-(4,4,5,5-Tetramethyl-1,3,2-dioxaborolan-2-yl)-1H-pyrazole (0.366 g, 1.2 mmol, 1.5 eq) was added to a solution (4-((5-bromo-1H-pyrrolo[2,3-b]pyridin-1-yl)methyl)piperidin-1-yl)(phenyl)methanone (0.5 g, 1.2 mmol, 1.0 eq) in a mixture of 1,4-dioxane and water (9:1) (3.6 mL) at room temperature. Then Na₂CO₃ (0.382 g, 3.6 mmol, 3.0 eq) was added and the mixture was purged with argon for 20 min. PdCl₂(dppf)-dichloromethane (0.102 g, 0.12 mmol, 0.1 eq) was added, the mixture purged with argon for a further 10 min and the mixture was then heated at 110-120° C. for 16 hr. After completion of the reaction (monitored by thin layer chromatography, 7% methanol in dichloromethane R_{f}=0.2), the mixture was cooled to room temperature and filtered through a celite bed. The filtrate was concentrated under reduced pressure and the crude product was purified by column chromatography on silica gel, eluting with a 3-4% gradient of methanol in dichloromethane to afford (4-((5-(1H-pyrazol-4-yl)-1H-pyrrolo[2,3-c]pyridin-1-yl)methyl)piperidin-1-yl)(phenyl)methanone (0.015 g, 3%) as an off white solid. LCMS: purity 95%; (ES⁺): m/z 386.28 (M+H⁺); tr=1.23 min.

¹H NMR (DMSO-d6, 400 MHz) δ: 12.45 (s, 1H), 8.8 (s, 1H), 8.16 (s, 1H), 7.99 (s, 1H), 7.79 (s, 1H), 7.55 (s, 1H), 7.44-7.42 (m, 3H), 7.35 (m, 2H), 6.45-6.44 (d, J=2.4 Hz, 1H) 4.5 (bs, 1H), 4.21-4.19 (d, J=7.2 Hz, 2H), 3.56 (bs, 1H), 2.96 (bs, 2H), 2.16 (bs, 1H), 1.54 (m, 2H), 1.28 (m, 2H).

### Examples 2-8

Using the above procedure and starting with the indicated starting material, the compounds shown in the table below were prepared:

| | Starting | | | |
|---|---|---|---|---|
| | Material | | Yield | LCMS and ¹H NMR (400 |
| Example | Intermediate | Name and Structure | (%) | MHz, DMSO-d₆) δ |
| 2 | 6 | 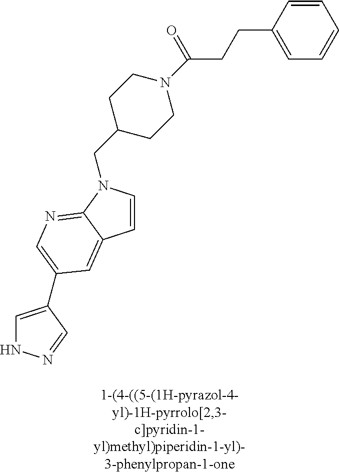 [SMILES: O=C(CCc1ccccc1)N1CCC(Cn2ccc3cc(-c4cn[nH]c4)cnc32)CC1] | 6.4 | Purity: 95.50%; (ES⁺): m/z 414.3 (M + H⁺); tr = 1.34 min. ¹H NMR (DMSO-d₆, 400 MHz) δ; 12.45 (s, 1H), 8.16 (s, 1H), 7.98 (s, 1H), 7.793 (s, 1H), 7.527 (s, 1H), 7.22 (m, 5H), 6.45-6.44 (d, J = 2.4, 1H), 4.40-4.37 (d, J = 12.4 Hz, 1H), 4.15-4.14 (d, J = 7.6 Hz, 1H), 3.86-3.82 (d, J = 13.6 Hz, 1H), 2.89-2.76 (m, 3H), 2.60-2.59 (m, 2H), 2.57- 2.42 (m, 1H), 2.09 (bs, 1H), 1.48-1.39 (m, 2H), 1.27-1.24 (m, 1H), 1.10-1.04 (m, 2H), 0.87- 0.84 (m, 1H). |
| 3 | 7 | 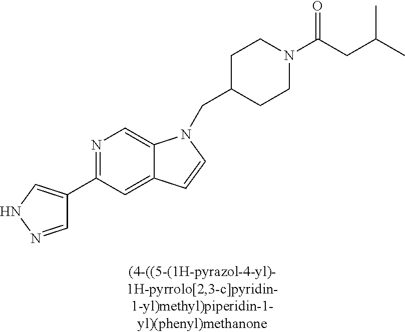 [SMILES: CC(C)CC(=O)N1CCC(Cn2ccc3cc(-c4cn[nH]c4)ncc32)CC1] | 7.4 | Purity: 98.59%; (ES⁺): m/z 366.31 (M + H⁺); tr = 1.39 min. ¹H NMR (DMSO-d₆, 400 MHz) δ; 12.93 (s, 1H), 8.84 (s, 1H), 8.16 (s, 1H), 7.99 (s, 1H), 7.79 (s, 1H), 7.54 (d, J = 2.8 Hz, 1H), 6.44 (d, J = 2.4 Hz, 1H), 4.40 (d, 11.6 Hz, 1H), 4.17 (d, J = 6.8 Hz) 2H), 3.86 (d, 1H), 2.89 (t, 1H), 2.50-2.41 (m, 1H), 2.15-2.14 (m, 3H), 1.98-1.91 (m, 1H), 1.46 (bd, 2H), 1.29-1.05 (m, 3H), 0.88-0.87 (d, J = 5.6 Hz, 6H). |
| 4 | 8 | 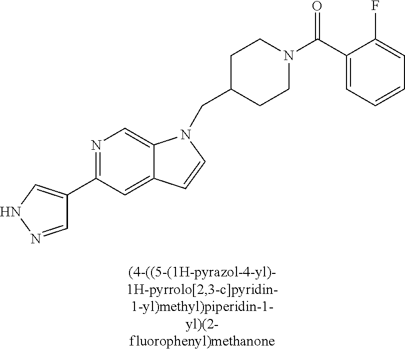 [SMILES: O=C(c1ccccc1F)N1CCC(Cn2ccc3cc(-c4cn[nH]c4)ncc32)CC1] | 8.5 | Purity: 99.17%; (ES⁺): m/z 404.26 (M + H⁺); tr = 1.24 min. ¹H NMR (CDCl₃, 400 MHz) δ; 8.74 (s, 1H), 8.12 (s, 2H), 7.75 (s, 1H), 7.41-7.31 (m, 2H), 7.21- 7.17 (m, 3H), 7.07 (t, 1H), 6.50 (d, J = 14.8 Hz, 1H), 4.84 (d, J = 13.2 Hz, 1H), 4.15-4.1. (m, 2H), 3.56 (d, J = 13.2 Hz, 1H), 2.96 (bs, 1H), 2.70 (t, 1H), 2.17 (m, 1H), 1.77 (d, J = 12.4 Hz, 2H), 1.41 (m, 2H). |
| 5 | 9 | 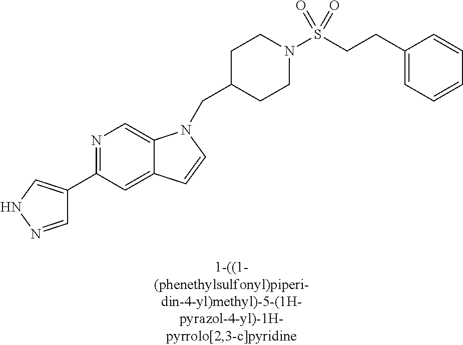 [SMILES: O=S(=O)(CCc1ccccc1)N1CCC(Cn2ccc3cc(-c4cn[nH]c4)ncc32)CC1] | 8.4 | Purity: 98.69%; (ES⁺): m/z 450.27 (M + H⁺); tr = 2.00 min ¹H NMR (CDCl₃, 400 MHz) δ; 8.72 (s, 1H), 8.12 (s, 1H), 7.75 (s, 1H), 7.33-7.29 (m, 3H), 7.24- 7.17 (m, 4H) 6.51 (d, J = 2.8 Hz, 1H), 4.10 (d, J = 7.6 Hz, 2H), 3.83 (d, J = 12.4 Hz, 2H), 3.15- 3.08 (m, 4H), 2.65 (t, J = 23.2 Hz, 2H), 2.04-2.01 (m, 1H), 1.66- 1.57 (m, 2H), 1.44-1.33 (m, 2H). |
| 6 | 10 | 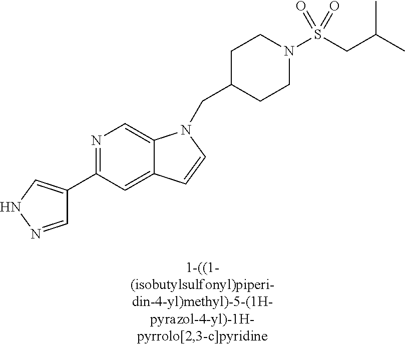 [SMILES: CC(C)CS(=O)(=O)N1CCC(Cn2ccc3cc(-c4cn[nH]c4)ncc32)CC1] | 5 | Purity: 96.30%; (ES⁺): m/z 402.28 (M + H⁺); tr = 1.31 min ¹H NMR (DMSO-d₆, 400 MHz) δ; 12.94 (s, 1H), 8.85 (s, 1H), 8.17 (s, 1H), 7.99 (s, 1H), 7.79 (s, 1H), 7.55 (d, J = 3.2 Hz, 1H), 6.44 (d, J = 2.4 Hz, 1H), 4.21 (d, J = 6.8 Hz, 2H), 3.57 (d, J = 11.6 Hz, 2H), 2.83 (d, J = 6.4 Hz, 2H), 2.67 (t, J = 22 Hz, 2H), 2.11-2.05 (m, 2H), 1.53 (d, J = 11.2 Hz, 2H), 1.32-1.24 (m, 2H), 1.00 (d, J = 6.8 Hz, 6H). |
| 7 | 11 | 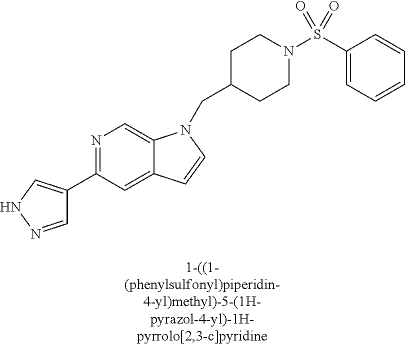 [SMILES: O=S(=O)(c1ccccc1)N1CCC(Cn2ccc3cc(-c4cn[nH]c4)ncc32)CC1] | 6.3 | Purity: 97.60%; (ES⁺): m/z 422.25 (M + H⁺); tr = 1.38 min ¹H NMR (CDCl₃, 400 MHz) δ; 8.64 (s, 1H), 8.09 (s, 2H), 7.72- 7.71 (m, 3H), 7.59-7.48 (m, 3H), 7.13 (d, J = 2.8 Hz, 1H), 6.46 (d, J = 3.2, 1H), 4.066 (d, J = 7.2 Hz, 2H), 3.83 (d, J = 1.2 Hz, 2H), 2.18 (t, J = 22.8 Hz, 2H), 1.84- 1.83 (m, 1H), 1.63-1.56 (m, 2H), 1.49-1.40 (m, 2H). |
| 8 | 12 | 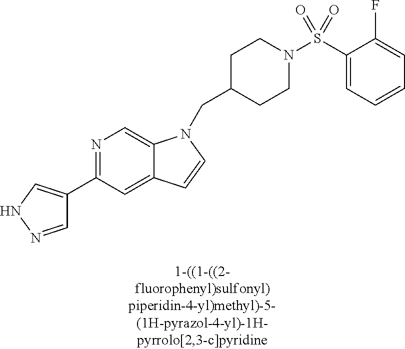 [SMILES: O=S(=O)(c1ccccc1F)N1CCC(Cn2ccc3cc(-c4cn[nH]c4)ncc32)CC1] | 6.9 | Purity: 99.38%; (ES⁺): m/z 440.24 (M + H⁺); tr = 1.37 min ¹H NMR (CDCl₃, 400 MHz) δ; 8.67 (s, 1H), 8.10 (s, 2H), 7.81 (t, 1H), 7.73 (s, 1H), 7.58-7.52 (m, 2H), 7.26-7.15 (m, 3H), 6.48 (d, J = 3.2 Hz, 1H), 4.10 (d, J = 7.2 Hz, 2H), 3.90 (d, J = 12.4 Hz, 2H), 2.47 (t, 2H), 1.96-1.92 (m, 1H), 1.64-1.56 (m, 2H), 1.45-1.38 (m, 2H). |

### Example 9

### Synthesis of (4-((5-(1H-pyrazol-4-yl)-1H-pyrrolo[2,3-b]pyridin-1-yl)methyl)piperidin-1-yl)(phenyl)methanone

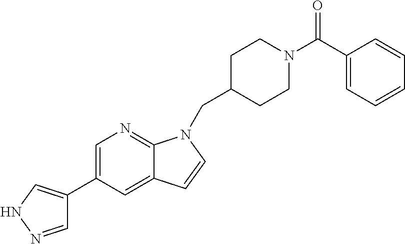 [SMILES: O=C(c1ccccc1)N1CCC(Cn2ccc3cc(-c4cn[nH]c4)cnc32)CC1]

Commercially available 4-(4,4,5,5-tetramethyl-1,3,2-dioxaborolan-2-yl)-1H-pyrazole (0.365 g, 1.8 mmol, 1.5 eq) was added to a solution (4-((5-bromo-1H-pyrrolo[2,3-b]pyridin-1-yl)methyl)piperidin-1-yl)(phenyl)methanone (0.5 g, 1.2 mmol, 1.0 eq) in a mixture of 1,4-dioxane and water (9:1) (3.65 mL) at room temperature. Then Na₂CO₃ was added (0.399 g, 3.0 mmol, 3.0 eq) and the mixture was purged with argon for 20 min. PdCl₂(dppf) dichloromethane (0.102 g, 0.12 mmol, 0.1 eq) was added, the reaction vessel purged with argon for a further 10 min and the mixture was then heated at 110° C.-120° C. for 16 hr. After completion of the reaction (monitored by thin layer chromatography, ethyl acetate R_{f}=0.4), the mixture was cooled to room temperature and filtered through a celite bed. The filtrate was concentrated under reduced pressure and the crude product was purified by column chromatography on silicagel, eluting with a 2-3% gradient of methanol in dichloromethane to afford (4-((5-(1H-pyrazol-4-yl)-1H-pyrrolo[2,3-b]pyridin-1-yl)methyl)piperidin-1-yl)(phenyl)methanone (0.13 g, 28%) as a brown solid. LCMS: purity 98.02%; (ES⁺): m/z 386.28 (M+H⁺); tr=1.38 min.

¹H NMR (CDCl₃, 400 MHz) δ:

8.48 (s, 1H), 8.08 (bs, 1H), 7.88 (bs, 1H), 7.87 (s, 2H), 7.37 (m, 5H), 7.17-7.16 (d, J=3.6 Hz, 1H), 6.46-6.45 (d, J=1.2 Hz, 1H), 4.70 (bs, 1H), 4.21 (bs, 2H), 3.76 (bs, 1H), 2.93-2.74 (m, 2H), 2.29-2.24 (m, 1H), 1.57 (m, 1H), 1.31-1.25 (m, 2H).

### Examples 10-16

Using the above procedure and starting with the indicated starting material, the compounds shown in the table below were prepared.

| | Starting | | | |
|---|---|---|---|---|
| | Material | | Yield | LCMS and ¹H NMR |
| Example | Intermediate | Name and Structure | (%) | (400 MHz, DMSO-d₆) δ |
| 10 | 5 | 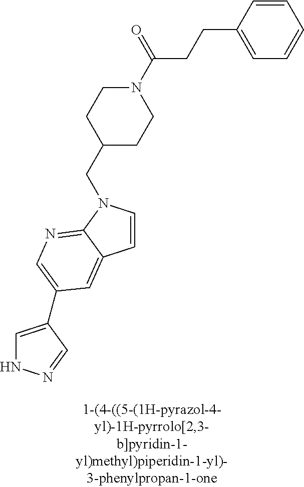 [SMILES: O=C(CCc1ccccc1)N1CCC(Cn2ccc3cc(-c4cn[nH]c4)cnc32)CC1] | 44.6 | Purity: 99.08%; (ES⁺): m/z 414.3 (M + H⁺); tr = 1.70 min. ¹H-NMR: 10.6-9.6 (s, 1H), 8.49-8.48 (d, J = 1.6 Hz, 1H), 8.01-8.00 (d, 1H), 7.88 (s, 2H), 7.30-7.14 (m, 6H), 6.46-6.45 (d, J = 3.6 Hz, 1H), 4.67-4.64 (d, 1H), 4.16-4.13 (m, 2H), 3.79-3.76 (d, 1H), 2.97-2.83 (m, 3H), 2.61-2.58 (m, 2H), 2.53-2.47 (t, 1H), 2.19-2.17 (m, 1H), 1.66-1.62 (m, 2H), 1.25- 1.20 (m, 1H), 1.07-1.04 (m, 1H). |
| 11 | 6 | 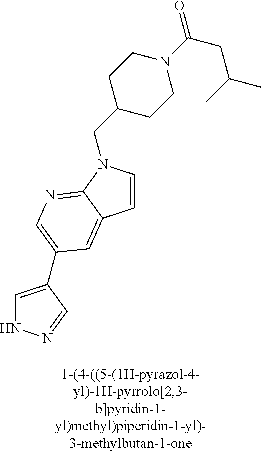 [SMILES: CC(C)CC(=O)N1CCC(Cn2ccc3cc(-c4cn[nH]c4)cnc32)CC1] | 46.6 | Purity: 98.7%; (ES⁺): m/z 366.31 (M + H⁺); tr = 1.39 min. ¹H-NMR: 8.49 (s, 1H), 8.01- 8.00 (d, J = 1.2 Hz, 1H), 7.88 (6s, 2H), 7.17-7.16 (d, J = 2.8 Hz, 1H), 6.46-6.45 (d, J = 3.6 Hz, 1H), 4.67-4.64 (d, 1H), 4.23-4.13 (, 2H), 3.89-3.85 (d, 1H), 2.98-2.92 (t, 1H), 2.53- 2.47 (t, 1H), 2.24-2.18 (m, 3H), 2.12-2.06 (m, 1H), 1.66-1.63 (bd, 2H), 1.28-1.18 (m, 3H), 0.96-0.947 (d, 6H). |
| 12 | 7 | 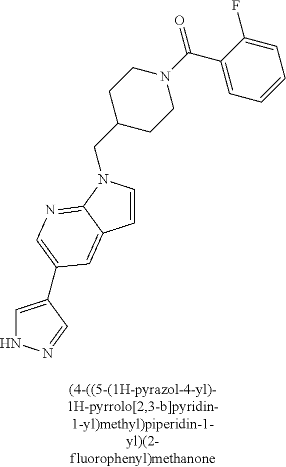 [SMILES: O=C(c1ccccc1F)N1CCC(Cn2ccc3cc(-c4cn[nH]c4)cnc32)CC1] | 35.5 | Purity: 97.34%; (ES⁺): m/z 404.26 (M + H⁺); tr = 1.43 min. ¹H-NMR: 11.0-9.6 (s, 1H), 8.49 (bs, 1H), 8.00 (bs, 1H), 7.88 (bs, 2H), 7.39-7.31 (m, 2H), 7.20-7.17 (m, 2H), 7.09-7.05 (t, 1H), 6.46-6.45 (d, J = 3.6 Hz, 1H), 4.79-4.76 (d, 2H), 4.26- 4.15 (m, 2H), 3.57-3.54 (m, 1H), 2.98 (m, 1H), 2.77-2.71 (t, 1H), 2.29-2.25 (m, 1H), 1.76- 1.73 (d, 1H), 1.42-1.36 (m, 3H). |
| 13 | 8 | 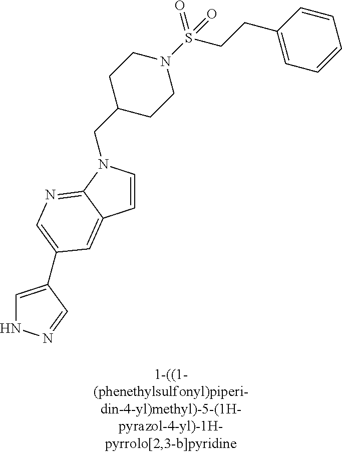 [SMILES: O=S(=O)(CCc1ccccc1)N1CCC(Cn2ccc3cc(-c4cn[nH]c4)cnc32)CC1] | 32.9 | Purity: 98.69%; (ES⁺): m/z 450.27 (M + H⁺); tr = 2.00 min ¹H-NMR: 10.7-9.7 (bs, 1H), 8.48 (s, 1H), 8.01-8.01 (d, 1H), 7.88 (s, 2H), 7.33-7.16 (m, 6H), 6.46-6.45 (d, J = 2.8 Hz, 1H), 4.20-4.18 (d, J = 7.2 Hz, 2H), 3.83-3.80 (d, 2H), 3.13-3.07 (m, 4H), 2.72-2.66 (t, 2H), 2.12 (m, 1H), 1.70-1.67 (d, 2H), 1.44-1.36 (m, 2H). |
| 14 | 9 | 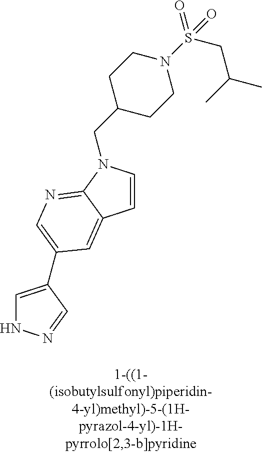 [SMILES: CC(C)CS(=O)(=O)N1CCC(Cn2ccc3cc(-c4cn[nH]c4)cnc32)CC1] | 21.0 | Purity: 99.30%; (ES⁺): m/z 402.28 (M + H⁺); tr = 1.71 min ¹H-NMR: 8.49 (s, 1H), 8.01- 8.01 (d, J = Hz, 1H), 7.88 (s, 2H), 7.17-7.16 (d, J = 3.6 Hz, 1H), 6.47-6.46 (d, J = 3.6 Hz, 1H), 4.20-4.18 (d, 2H), 3.81- 3.78 (d, 2H), 2.72-2.64 (m, 4H), 2.29-2.22 (m, 1H), 2.15- 2.10 (m, 1H), 1.70-1.67 (d, 2H), 1.47-1.37 (m, 2H), 1.09- 1.08 (d, 6H). |
| 15 | 10 | 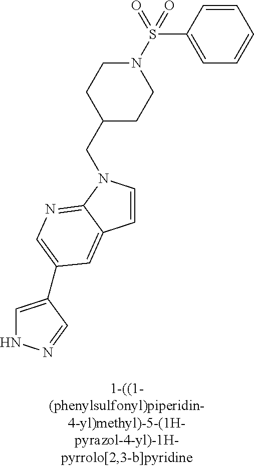 [SMILES: O=S(=O)(c1ccccc1)N1CCC(Cn2ccc3cc(-c4cn[nH]c4)cnc32)CC1] | 51.4 | Purity: 99.60%; (ES⁺): m/z 422.25 (M + H⁺); tr = 1.79 min ¹H-NMR: 10.7-9.71 (bs, 1H), 8.44-8.44 (d, J = 1.6 Hz, 1H), 7.98-7.98 (d, 1H, J = 6 Hz, 1H), 7.86 (s, 2H), 7.73-7.71 (d, 2H), 7.59-7.48 (m, 3H), 7.12-7.11 (d, J = 3.6, 1H), 6.43-6.42 (d, J = 3.2 Hz, 1H), 4.15-4.13 (d, J = 7.2 Hz, 2H), 3.82-3.79 (d, J = 11.6 Hz, 2H), 2.24-2.18 (m, 2H), 1.96-1.91 (m, 1H), 1.66- 1.63 (d, 2H), 1.49-1.38 (m, 2H). |
| 16 | 11 | 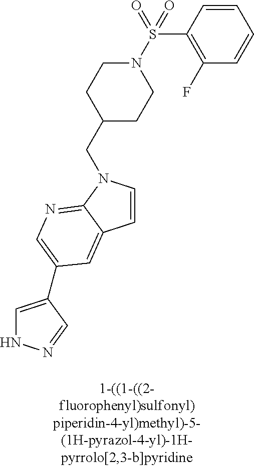 [SMILES: O=S(=O)(c1ccccc1F)N1CCC(Cn2ccc3cc(-c4cn[nH]c4)cnc32)CC1] | 47.9 | Purity: 99.38%; (ES⁺): m/z 440.24 (M + H⁺); tr = 1.83 min ¹H-NMR: 10.7-9.5 (bs, 1H), 8.455 (s, 1H), 7.990 (s, 1H), 7.86 (s, 1H), 7.83-7.80 (d, 2H), 7.57-7.52 (m, 1H), 7.37.13 (m, 3H), 6.44-6.43 (d, J = 3.6 Hz, 1H) 4.17-4.15 (d, 2H), 3.90- 3.877 (d, 2H), 2.53-2.47 (t, 2H), 2.0717-2.043 (m, 1H), 1.679-1.648 (m, 2H), 1.47- 1.371 (m, 2H). |

### Examples 17-25

Reaction of 1-(piperidin-4-ylmethyl)-5-(1-(tetrahydro-2H-pyran-2-yl)-1H-pyrazol-4-yl)-1H-pyrrolo[2,3-c]pyridine (Intermediate 31) and the acid chlorides shown in the table below using the general procedure described in Intermediate 6, followed by deprotection by treatment with methanolic p-toluene sulfonic acid at room temperature, the following can be prepared:

| | Acyl | |
|---|---|---|
| Example | Chloride | Product |
| 17 | 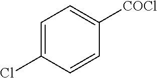 | 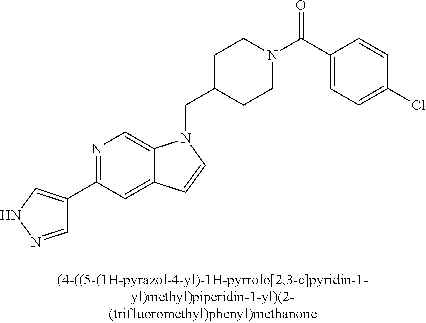 [SMILES: O=C(c1ccc(Cl)cc1)N1CCC(Cn2ccc3cc(-c4cn[nH]c4)ncc32)CC1] |
| 18 | 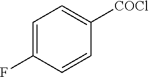 | 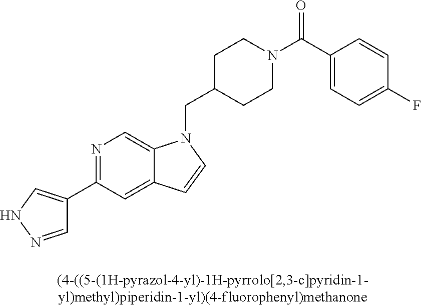 [SMILES: O=C(c1ccc(F)cc1)N1CCC(Cn2ccc3cc(-c4cn[nH]c4)ncc32)CC1] |
| 19 | 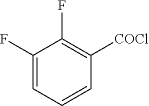 [SMILES: O=C(Cl)c1cccc(F)c1F] | 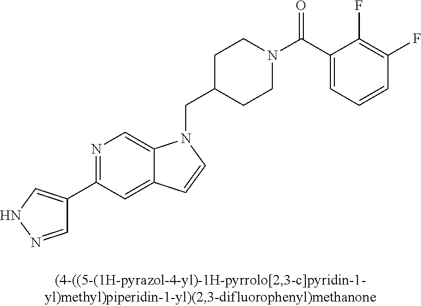 [SMILES: O=C(c1cccc(F)c1F)N1CCC(Cn2ccc3cc(-c4cn[nH]c4)ncc32)CC1] |
| 20 | 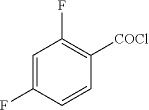 [SMILES: O=C(Cl)c1ccc(F)cc1F] | 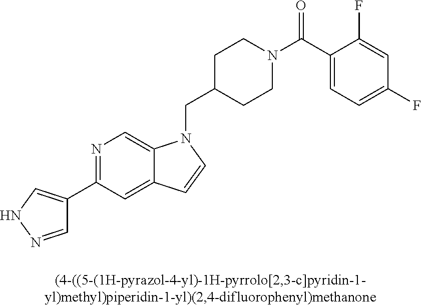 [SMILES: O=C(c1ccc(F)cc1F)N1CCC(Cn2ccc3cc(-c4cn[nH]c4)ncc32)CC1] |
| 21 | 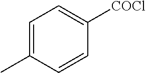 | 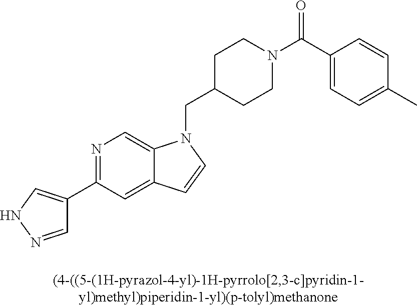 [SMILES: Cc1ccc(C(=O)N2CCC(Cn3ccc4cc(-c5cn[nH]c5)ncc43)CC2)cc1] |
| 22 | 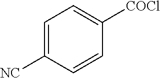 [SMILES: N#Cc1ccc(C(=O)Cl)cc1] | 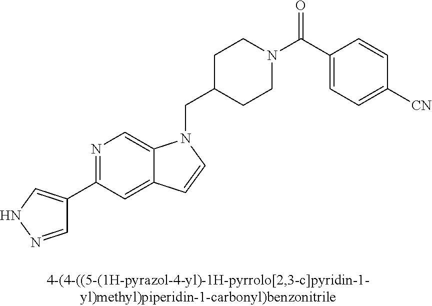 [SMILES: N#Cc1ccc(C(=O)N2CCC(Cn3ccc4cc(-c5cn[nH]c5)ncc43)CC2)cc1] |
| 23 | 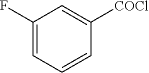 | 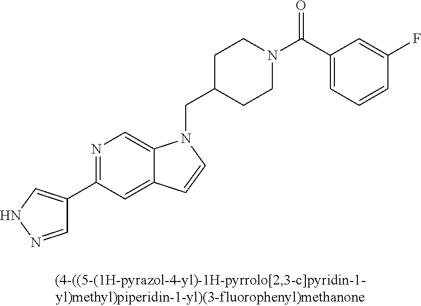 [SMILES: O=C(c1cccc(F)c1)N1CCC(Cn2ccc3cc(-c4cn[nH]c4)ncc32)CC1] |
| 24 | 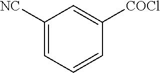 [SMILES: N#Cc1cccc(C(=O)Cl)c1] | 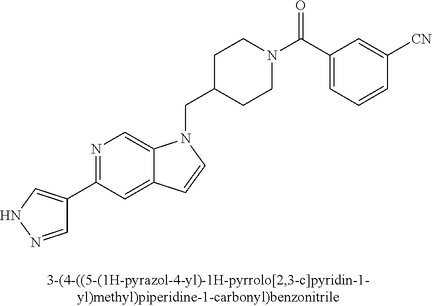 [SMILES: N#Cc1cccc(C(=O)N2CCC(Cn3ccc4cc(-c5cn[nH]c5)ncc43)CC2)c1] |
| 25 | 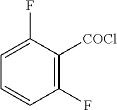 [SMILES: O=C(Cl)c1c(F)cccc1F] | 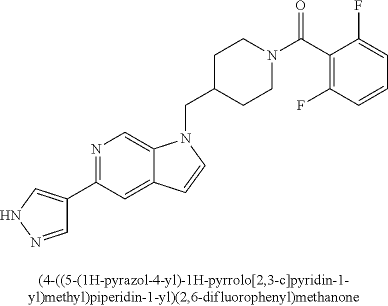 [SMILES: O=C(c1c(F)cccc1F)N1CCC(Cn2ccc3cc(-c4cn[nH]c4)ncc32)CC1] |

### Examples 26-33

Reaction of 1-(piperidin-4-ylmethyl)-5-(1-(tetrahydro-2H-pyran-2-yl)-1H-pyrazol-4-yl)-1H-pyrrolo[2,3-c]pyri dine (Intermediate 31) and the acid chlorides shown in the table below using the general procedure described in Intermediate 6, followed by deprotection by treatment with methanolic p-toluene sulfonic acid at room temperature, the following can be prepared:

| Example | Acyl Chloride | Product |
|---|---|---|
| 26 | 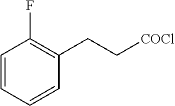 [SMILES: O=C(Cl)CCc1ccccc1F] | 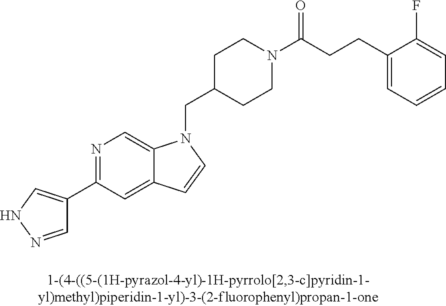 [SMILES: O=C(CCc1ccccc1F)N1CCC(Cn2ccc3cc(-c4cn[nH]c4)ncc32)CC1] |
| 27 | 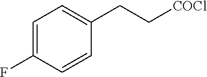 [SMILES: O=C(Cl)CCc1ccc(F)cc1] | 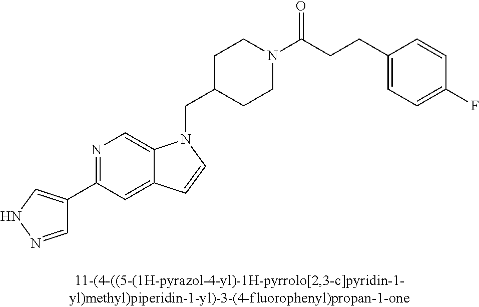 [SMILES: O=C(CCc1ccc(F)cc1)N1CCC(Cn2ccc3cc(-c4cn[nH]c4)ncc32)CC1] |
| 28 | 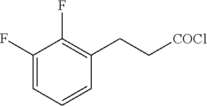 [SMILES: O=C(Cl)CCc1cccc(F)c1F] | 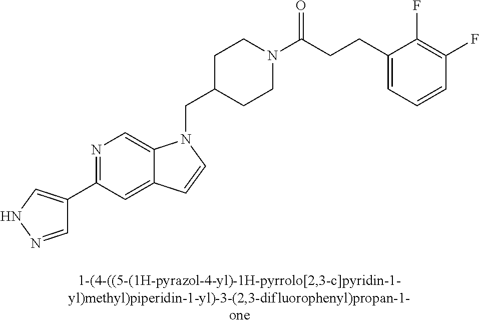 [SMILES: O=C(CCc1cccc(F)c1F)N1CCC(Cn2ccc3cc(-c4cn[nH]c4)ncc32)CC1] |
| 29 | 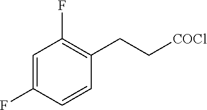 [SMILES: O=C(Cl)CCc1ccc(F)cc1F] | 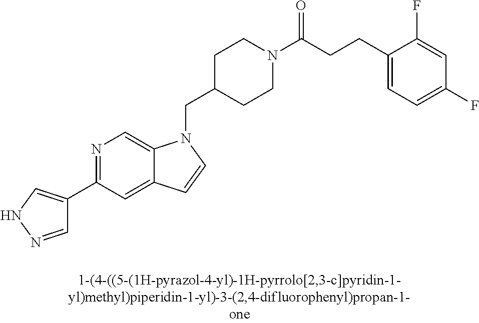 [SMILES: O=C(CCc1ccc(F)cc1F)N1CCC(Cn2ccc3cc(-c4cn[nH]c4)ncc32)CC1] |
| 30 | 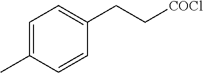 | 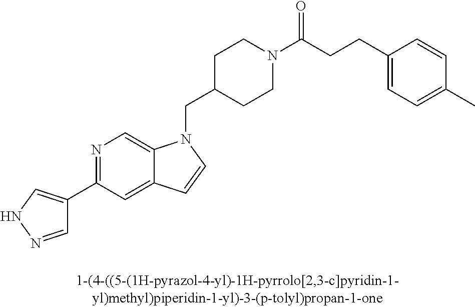 [SMILES: Cc1ccc(CCC(=O)N2CCC(Cn3ccc4cc(-c5cn[nH]c5)ncc43)CC2)cc1] |
| 31 | 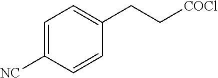 [SMILES: N#Cc1ccc(CCC(=O)Cl)cc1] | 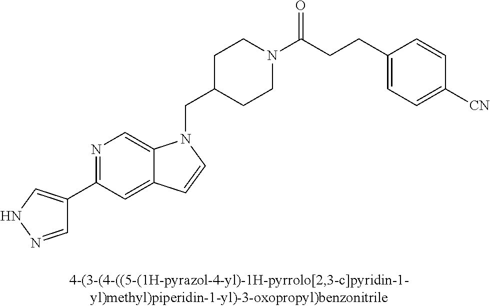 [SMILES: N#Cc1ccc(CCC(=O)N2CCC(Cn3ccc4cc(-c5cn[nH]c5)ncc43)CC2)cc1] |
| 32 | 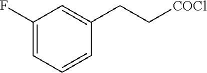 [SMILES: O=C(Cl)CCc1cccc(F)c1] | 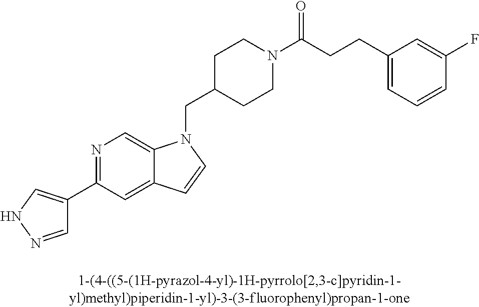 [SMILES: O=C(CCc1cccc(F)c1)N1CCC(Cn2ccc3cc(-c4cn[nH]c4)ncc32)CC1] |
| 33 | 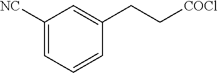 [SMILES: N#Cc1cccc(CCC(=O)Cl)c1] | 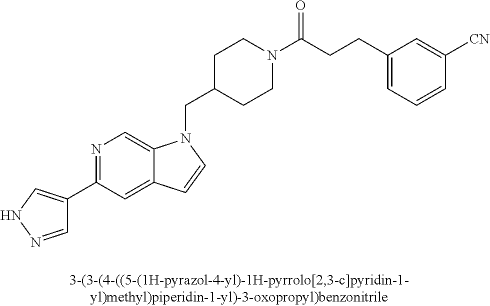 [SMILES: N#Cc1cccc(CCC(=O)N2CCC(Cn3ccc4cc(-c5cn[nH]c5)ncc43)CC2)c1] |

### Examples 34-39

Reaction of 1-((5,5-dimethylpyrrolidin-3-yl)methyl)-5-(1-(tetrahydro-2H-pyran-2-yl)-1H-pyrazol-4-yl)-1H-pyrrolo[2,3-c]pyridine (Intermediate 43) and the acid chlorides shown in the table below using the general procedure described in Intermediate 6, followed by deprotection by treatment with methanolic p-toluene sulfonic acid at room temperature, the following can be prepared:

| | Acyl | |
|---|---|---|
| Example | Chloride | Product |
| 34 | 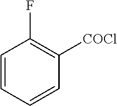 | 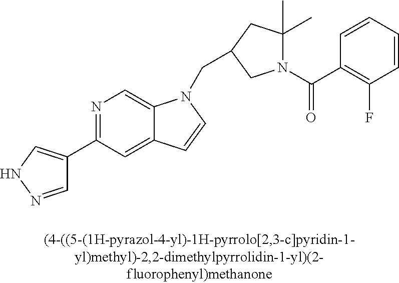 [SMILES: CC1(C)CC(Cn2ccc3cc(-c4cn[nH]c4)ncc32)CN1C(=O)c1ccccc1F] |
| 35 | 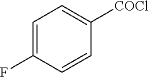 | 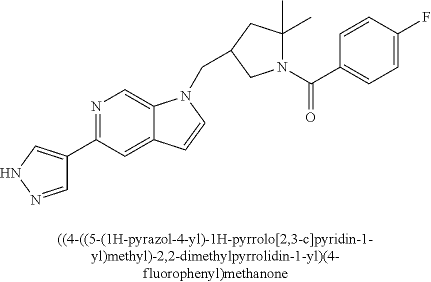 [SMILES: CC1(C)CC(Cn2ccc3cc(-c4cn[nH]c4)ncc32)CN1C(=O)c1ccc(F)cc1] |
| 36 | 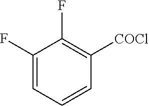 [SMILES: O=C(Cl)c1cccc(F)c1F] | 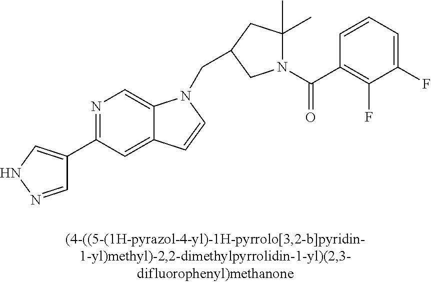 [SMILES: CC1(C)CC(Cn2ccc3cc(-c4cn[nH]c4)ncc32)CN1C(=O)c1cccc(F)c1F] |
| 37 | 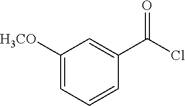 [SMILES: COc1cccc(C(=O)Cl)c1] | 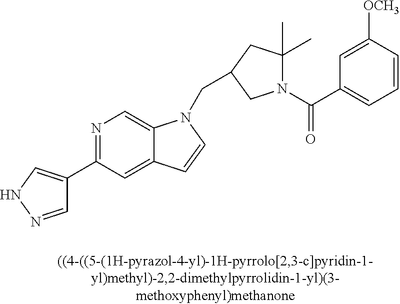 [SMILES: COc1cccc(C(=O)N2CC(Cn3ccc4cc(-c5cn[nH]c5)ncc43)CC2(C)C)c1] |
| 38 | 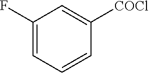 | 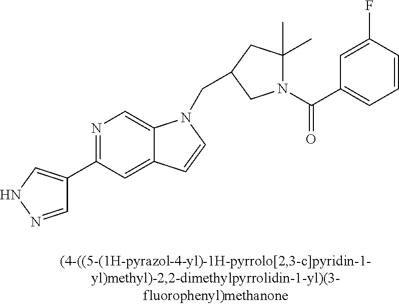 [SMILES: CC1(C)CC(Cn2ccc3cc(-c4cn[nH]c4)ncc32)CN1C(=O)c1cccc(F)c1] |
| 39 | 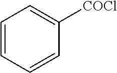 | 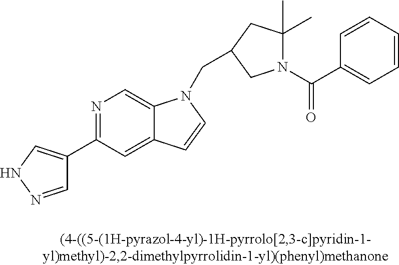 [SMILES: CC1(C)CC(Cn2ccc3cc(-c4cn[nH]c4)ncc32)CN1C(=O)c1ccccc1] |

### Examples 40-41

Reaction of 1-((5,5-dimethylpyrrolidin-3-yl)methyl)-5-(1-(tetrahydro-2H-pyran-2-yl)-1H-pyrazol-4-yl)-1H-pyrrolo[2,3-c]pyridine (Intermediate 43) and the acid chlorides shown in the table below using the general procedure described in Intermediate 6, followed by deprotection by treatment with methanolic p-toluene sulfonic acid at room temperature, the following can be prepared:

| Example | Acyl Chloride | Product |
|---|---|---|
| 40 | 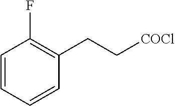 [SMILES: O=C(Cl)CCc1ccccc1F] | 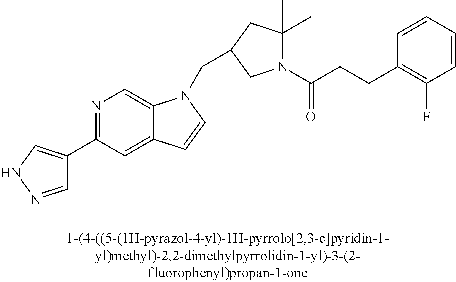 [SMILES: CC1(C)CC(Cn2ccc3cc(-c4cn[nH]c4)ncc32)CN1C(=O)CCc1ccccc1F] |
| 41 | 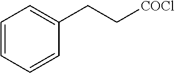 [SMILES: O=C(Cl)CCc1ccccc1] | 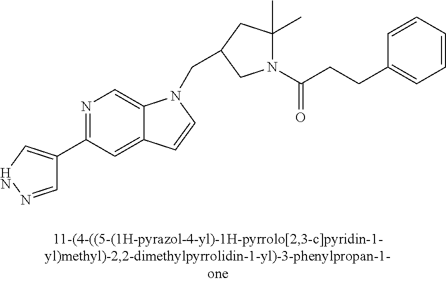 [SMILES: CC1(C)CC(Cn2ccc3cc(-c4cn[nH]c4)ncc32)CN1C(=O)CCc1ccccc1] |

### Examples 42-45

Reaction of rac.-cis-1-((5-methylpyrrolidin-3-yl)methyl)-5-(1-(tetrahydro-2H-pyran-2-yl)-1H-pyrazol-4-yl)-1H-pyrrolo[2,3-c]pyridine (Intermediate 44) and the acid chlorides shown in the table below using the general procedure described in Intermediate 6, followed by deprotection by treatment with methanolic p-toluene sulfonic acid at room temperature, the following can be prepared:

| | Acyl | |
|---|---|---|
| Example | Chloride | Product |
| 42 | 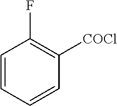 | 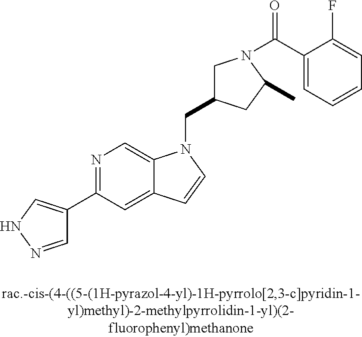 |
| 43 | 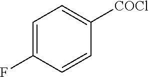 | 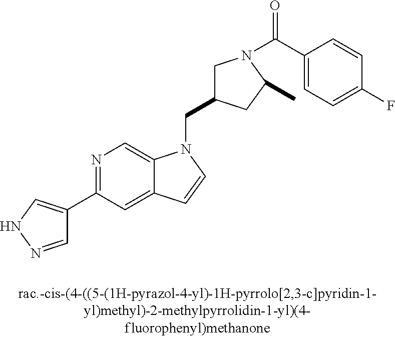 |
| 44 | 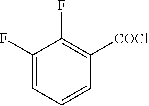 [SMILES: O=C(Cl)c1cccc(F)c1F] | 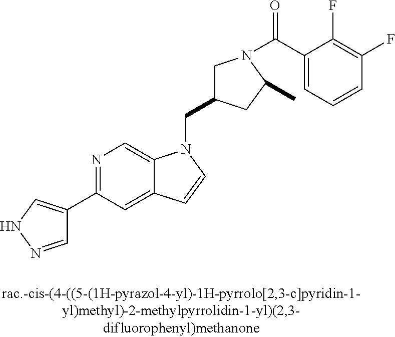 |
| 45 | 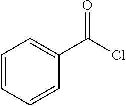 | 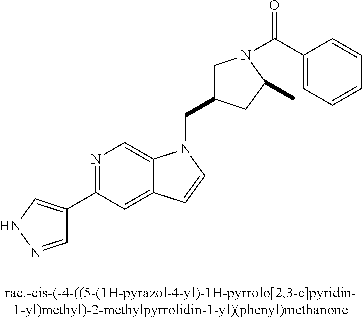 |

### Examples 46-49

Reaction of rac.-trans-1-((5-methylpyrrolidin-3-yl)methyl)-5-(1-(tetrahydro-2H-pyran-2-yl)-1H-pyrazol-4-yl)-1H-pyrrolo[2,3-c]pyridine (Intermediate 45) and the acid chlorides shown in the table below using the general procedure described in Intermediate 6, followed by deprotection by treatment with methanolic p-toluene sulfonic acid at room temperature, the following can be prepared:

| | Acyl | |
|---|---|---|
| Example | Chloride | Product |
| 46 | 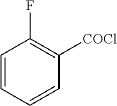 | 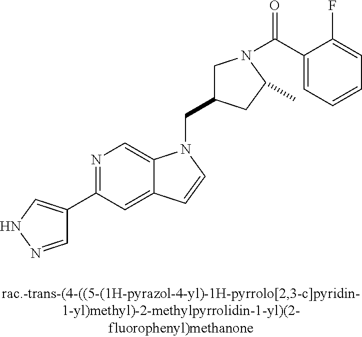 |
| 47 | 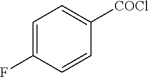 | 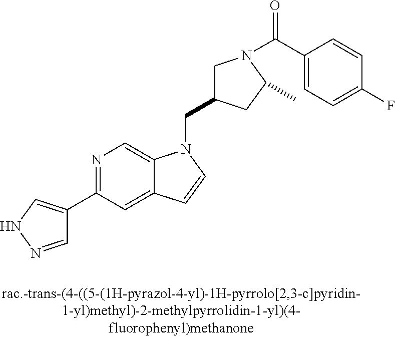 |
| 48 | 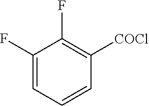 [SMILES: O=C(Cl)c1cccc(F)c1F] | 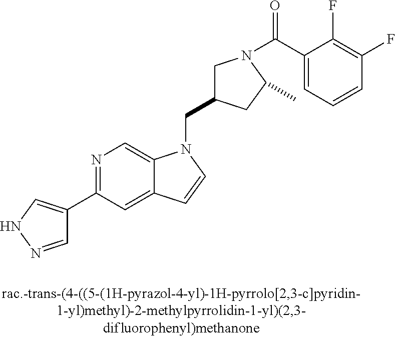 |
| 49 | 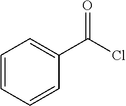 | 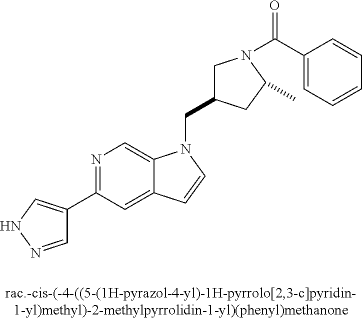 |

### Examples 50-51

Reaction of rac.-cis-1-((5-methylpyrrolidin-3-yl)methyl)-5-(1-(tetrahydro-2H-pyran-2-yl)-1H-pyrazol-4-yl)-1H-pyrrolo[2,3-c]pyridine (Intermediate 44) and the acid chlorides shown in the table below using the general procedure described in Intermediate 6, followed by deprotection by treatment with methanolic p-toluene sulfonic acid at room temperature, the following can be prepared:

| Example | Acyl Chloride | Product |
|---|---|---|
| 50 | 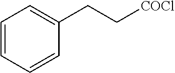 [SMILES: O=C(Cl)CCc1ccccc1] | 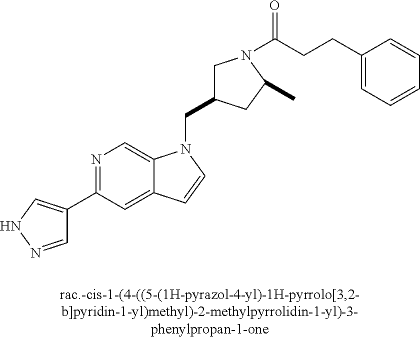 |
| 51 | 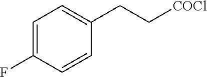 [SMILES: O=C(Cl)CCc1ccc(F)cc1] | 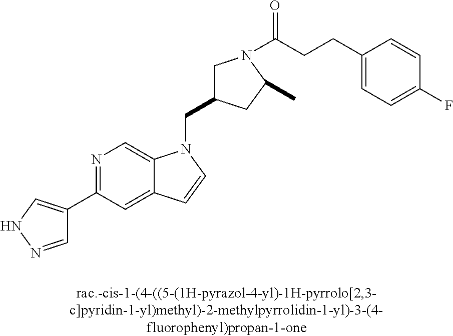 |

### Examples 52-53

Reaction of rac.-trans-1-((5-methylpyrrolidin-3-yl)methyl)-5-(1-(tetrahydro-2H-pyran-2-yl)-1H-pyrazol-4-yl)-1H-pyrrolo[2,3-c]pyridine (Intermediate 45) and the acid chlorides shown in the table below using the general procedure described in Intermediate 6, followed by deprotection by treatment with methanolic p-toluene sulfonic acid at room temperature, the following can be prepared:

| Example | Acyl Chloride | Product |
|---|---|---|
| 52 | 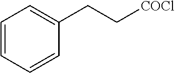 [SMILES: O=C(Cl)CCc1ccccc1] | 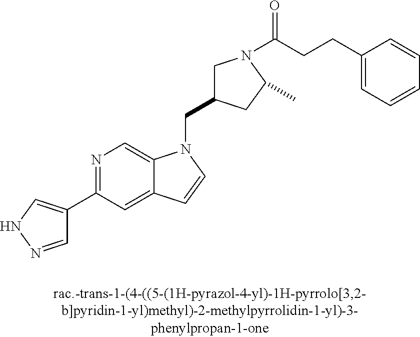 |
| 53 | 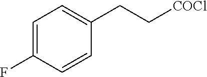 [SMILES: O=C(Cl)CCc1ccc(F)cc1] | 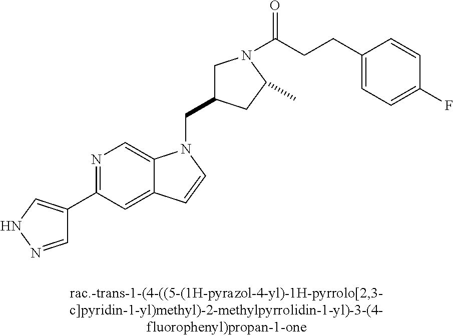 |

### Examples 54-56

By reaction of 1-((5-methylpyrrolidin-3-yl)methyl)-5-(1-(tetrahydro-2H-pyran-2-yl)-1H-pyrazol-4-yl)-1H-pyrrolo[2,3-c]pyridine (Intermediate 42) and the acid chlorides shown in the table below using the general procedure described in Intermediate 6, followed by deprotection by treatment with methanolic p-toluene sulfonic acid at room temperature, the following can be prepared:

| | Acyl | |
|---|---|---|
| Example | Chloride | Product |
| 54 | 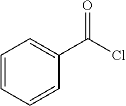 | 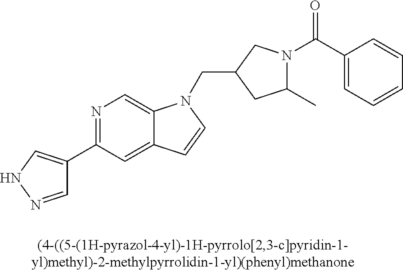 [SMILES: CC1CC(Cn2ccc3cc(-c4cn[nH]c4)ncc32)CN1C(=O)c1ccccc1] |
| 55 | 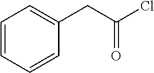 | 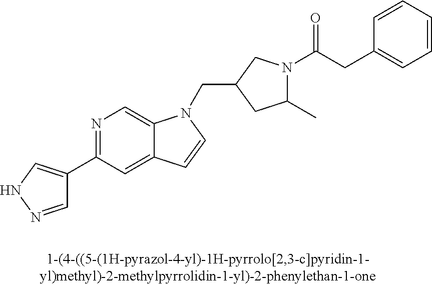 [SMILES: CC1CC(Cn2ccc3cc(-c4cn[nH]c4)ncc32)CN1C(=O)Cc1ccccc1] |
| 56 | 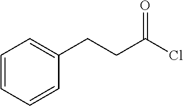 [SMILES: O=C(Cl)CCc1ccccc1] | 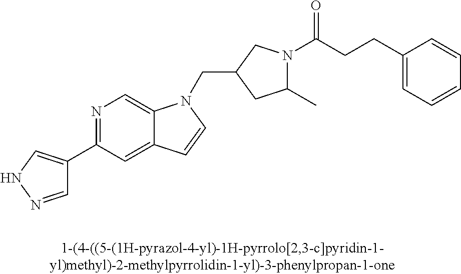 [SMILES: CC1CC(Cn2ccc3cc(-c4cn[nH]c4)ncc32)CN1C(=O)CCc1ccccc1] |

### Examples 57-58

By reaction of rac.-1-(azepan-4-ylmethyl)-5-(1-(tetrahydro-2H-pyran-2-yl)-1H-pyrazol-4-yl)-1H-pyrrolo[2,3-c]pyridine (Intermediate 46) and the acid chlorides shown in the table below using the general procedure described in Intermediate 6, followed by deprotection by treatment with methanolic p-toluene sulfonic acid at room temperature, the following can be prepared:

| | Acyl | |
|---|---|---|
| Example | Chloride | Product |
| 57 | 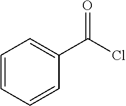 | 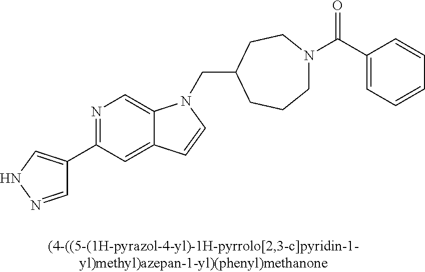 [SMILES: O=C(c1ccccc1)N1CCCC(Cn2ccc3cc(-c4cn[nH]c4)ncc32)CC1] |
| 58 | 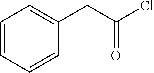 | 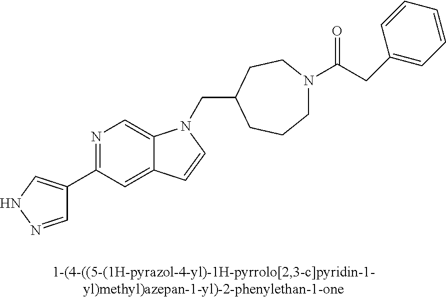 [SMILES: O=C(Cc1ccccc1)N1CCCC(Cn2ccc3cc(-c4cn[nH]c4)ncc32)CC1] |

### Examples 59-61

By reaction of rac.-benzyl 2-((5-(1-(tetrahydro-2H-pyran-2-yl)-1H-pyrazol-4-yl)-1H-pyrrolo[2,3-b]pyridin-1-yl)methyl)-7-azabicyclo[2.2.1]heptane-7-carboxylate (Intermediate 47) and the acid chlorides shown in the table below using the general procedure described in

Intermediate 6, followed by deprotection by treatment with methanolic p-toluene sulfonic acid at room temperature, the following can be prepared:

| | Acyl | |
|---|---|---|
| Example | Chloride | Product |
| 59 | 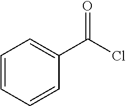 | 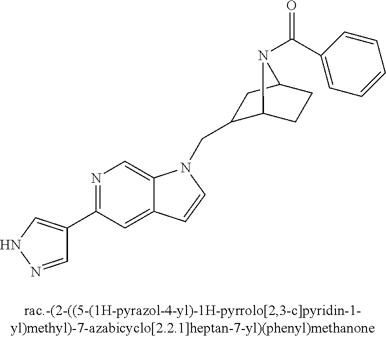 [SMILES: O=C(c1ccccc1)N1C2CCC1C(Cn1ccc3cc(-c4cn[nH]c4)ncc31)C2] |
| 60 | 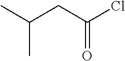 | 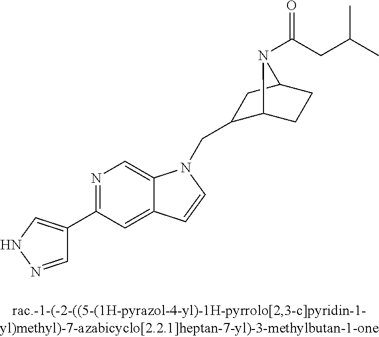 [SMILES: CC(C)CC(=O)N1C2CCC1C(Cn1ccc3cc(-c4cn[nH]c4)ncc31)C2] |
| 61 | 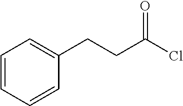 [SMILES: O=C(Cl)CCc1ccccc1] | 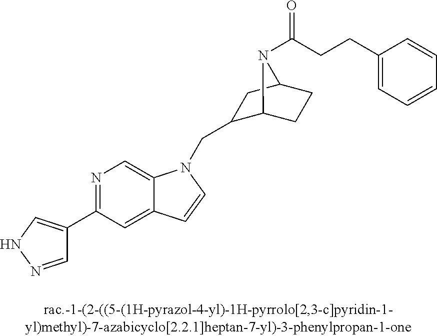 [SMILES: O=C(CCc1ccccc1)N1C2CCC1C(Cn1ccc3cc(-c4cn[nH]c4)ncc31)C2] |

### Example 62

### Synthesis of 1-(4-((5-(5-methyl-1H-pyrazol-4-yl)-1H-pyrrolo[2,3-c]pyridin-1-yl)methyl)piperidin-1-yl)-3-phenylpropan-1-one

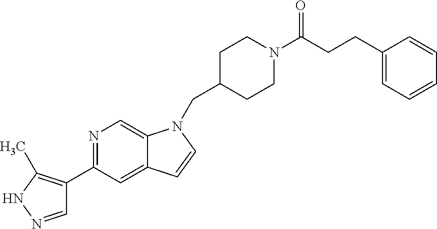 [SMILES: Cc1[nH]ncc1-c1cc2ccn(CC3CCN(C(=O)CCc4ccccc4)CC3)c2cn1]

By reaction of 3-phenyl-1-(4-((5-(4,4,5,5-tetramethyl-1,3,2-dioxaborolan-2-yl)-1H-pyrrolo[2,3-c]pyridin-1-yl)methyl)piperidin-1-yl)propan-1-one (Intermediate 63) and 4-bromo-5-methyl-1-(tetrahydro-2H-pyran-2-yl)-1H-pyrazole (Intermediate 24) using the general procedure described in example 1, followed by deprotection by treatment with methanolic p-toluene sulfonic acid at room temperature, 1-(4-((5-(5-methyl-1H-pyrazol-4-yl)-1H-pyrrolo[2,3-c]pyridin-1-yl)methyl)piperidin-1-yl)-3-phenylpropan-1-one can be prepared.

### Examples 63-71

Reaction of 1-(piperidin-4-ylmethyl)-5-(1-(tetrahydro-2H-pyran-2-yl)-1H-pyrazol-4-yl)-1H-pyrrolo[2,3-b]pyridine (Intermediate 48) and the acid chlorides shown in the table below using the general procedure described in Intermediate 16, followed by deprotection by treatment with methanolic p-toluene sulfonic acid at room temperature, the following can be prepared:

| | Acyl | |
|---|---|---|
| Example | Chloride | Product |
| 63 | 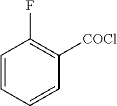 | 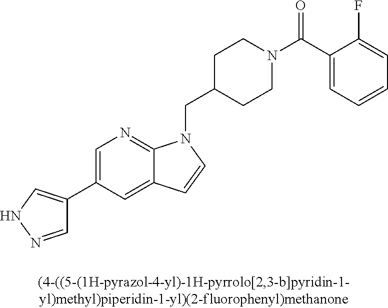 [SMILES: O=C(c1ccccc1F)N1CCC(Cn2ccc3cc(-c4cn[nH]c4)cnc32)CC1] |
| 64 | 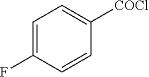 | 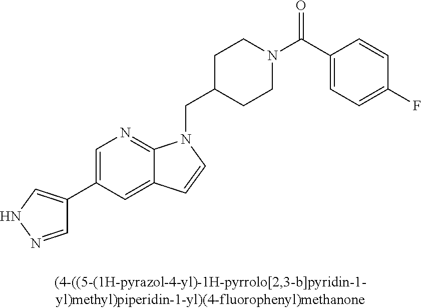 [SMILES: O=C(c1ccc(F)cc1)N1CCC(Cn2ccc3cc(-c4cn[nH]c4)cnc32)CC1] |
| 65 | 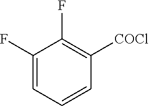 [SMILES: O=C(Cl)c1cccc(F)c1F] | 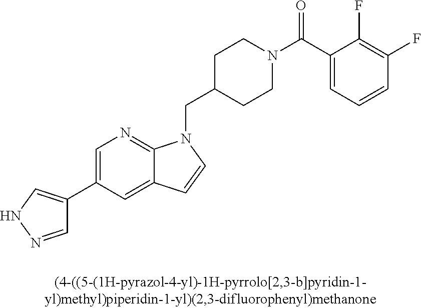 [SMILES: O=C(c1cccc(F)c1F)N1CCC(Cn2ccc3cc(-c4cn[nH]c4)cnc32)CC1] |
| 66 | 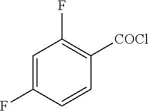 [SMILES: O=C(Cl)c1ccc(F)cc1F] | 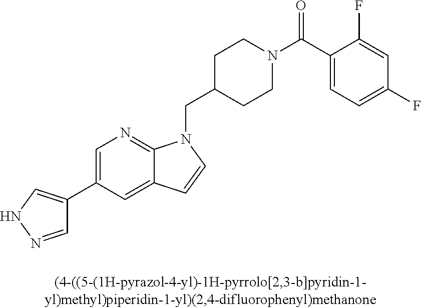 [SMILES: O=C(c1ccc(F)cc1F)N1CCC(Cn2ccc3cc(-c4cn[nH]c4)cnc32)CC1] |
| 67 | 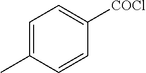 | 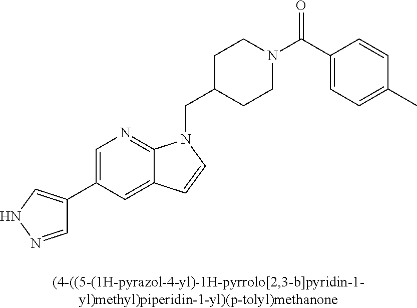 [SMILES: Cc1ccc(C(=O)N2CCC(Cn3ccc4cc(-c5cn[nH]c5)cnc43)CC2)cc1] |
| 68 | 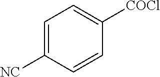 [SMILES: N#Cc1ccc(C(=O)Cl)cc1] | 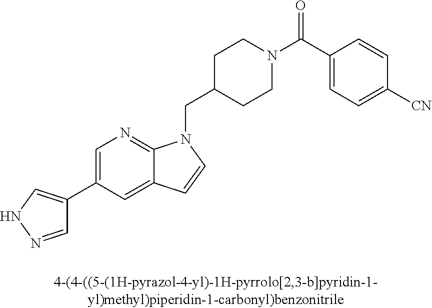 [SMILES: N#Cc1ccc(C(=O)N2CCC(Cn3ccc4cc(-c5cn[nH]c5)cnc43)CC2)cc1] |
| 69 | 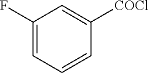 | 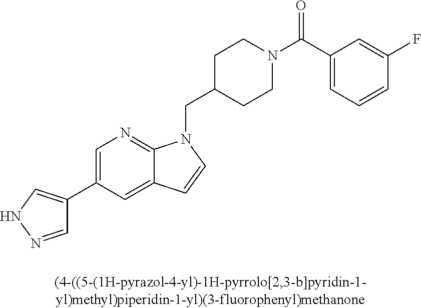 [SMILES: O=C(c1cccc(F)c1)N1CCC(Cn2ccc3cc(-c4cn[nH]c4)cnc32)CC1] |
| 70 | 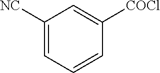 [SMILES: N#Cc1cccc(C(=O)Cl)c1] | 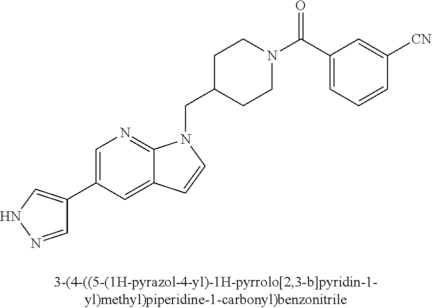 [SMILES: N#Cc1cccc(C(=O)N2CCC(Cn3ccc4cc(-c5cn[nH]c5)cnc43)CC2)c1] |
| 71 | 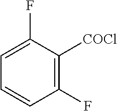 [SMILES: O=C(Cl)c1c(F)cccc1F] | 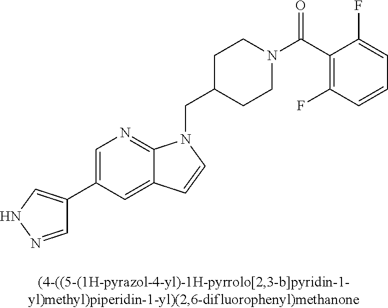 [SMILES: O=C(c1c(F)cccc1F)N1CCC(Cn2ccc3cc(-c4cn[nH]c4)cnc32)CC1] |

### Examples 72-79

Reaction of 1-(piperidin-4-ylmethyl)-5-(1-(tetrahydro-2H-pyran-2-yl)-1H-pyrazol-4-yl)-1H-pyrrolo[2,3-b]pyridine (Intermediate 48) and the acid chlorides shown in the table below using the general procedure described in Intermediate 16, followed by deprotection by treatment with methanolic p-toluene sulfonic acid at room temperature, the following can be prepared:

| Example | Acyl Chloride | Product |
|---|---|---|
| 72 | 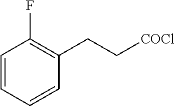 [SMILES: O=C(Cl)CCc1ccccc1F] | 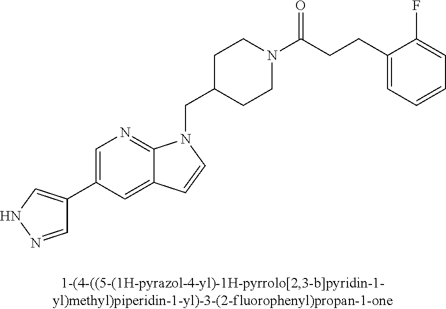 [SMILES: O=C(CCc1ccccc1F)N1CCC(Cn2ccc3cc(-c4cn[nH]c4)cnc32)CC1] |
| 73 | 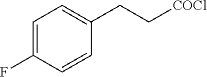 [SMILES: O=C(Cl)CCc1ccc(F)cc1] | 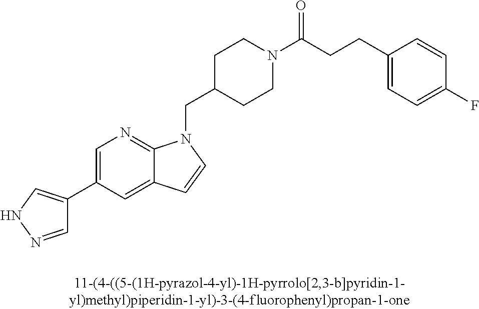 [SMILES: O=C(CCc1ccc(F)cc1)N1CCC(Cn2ccc3cc(-c4cn[nH]c4)cnc32)CC1] |
| 74 | 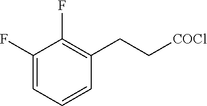 [SMILES: O=C(Cl)CCc1cccc(F)c1F] | 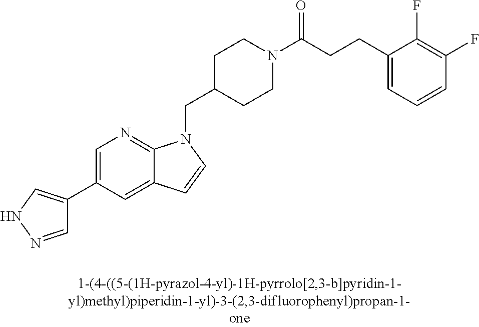 [SMILES: O=C(CCc1cccc(F)c1F)N1CCC(Cn2ccc3cc(-c4cn[nH]c4)cnc32)CC1] |
| 75 | 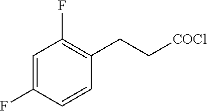 [SMILES: O=C(Cl)CCc1ccc(F)cc1F] | 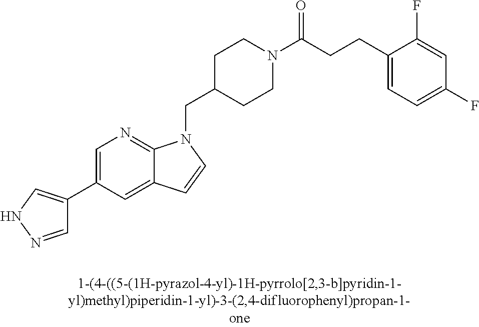 [SMILES: O=C(CCc1ccc(F)cc1F)N1CCC(Cn2ccc3cc(-c4cn[nH]c4)cnc32)CC1] |
| 76 | 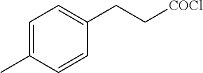 | 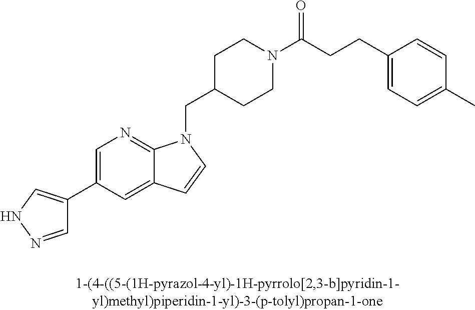 [SMILES: Cc1ccc(CCC(=O)N2CCC(Cn3ccc4cc(-c5cn[nH]c5)cnc43)CC2)cc1] |
| 77 | 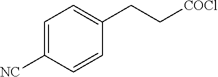 [SMILES: N#Cc1ccc(CCC(=O)Cl)cc1] | 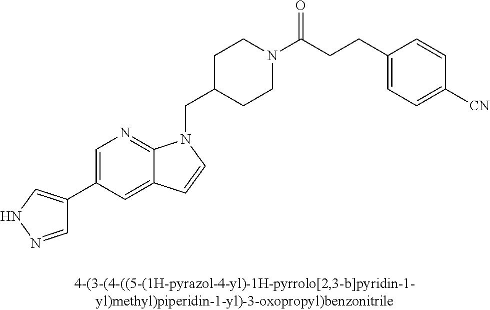 [SMILES: N#Cc1ccc(CCC(=O)N2CCC(Cn3ccc4cc(-c5cn[nH]c5)cnc43)CC2)cc1] |
| 78 | 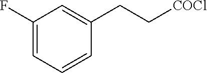 [SMILES: O=C(Cl)CCc1cccc(F)c1] | 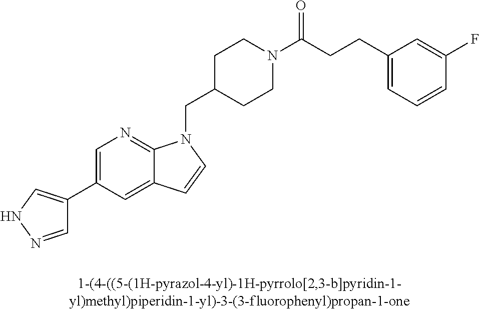 [SMILES: O=C(CCc1cccc(F)c1)N1CCC(Cn2ccc3cc(-c4cn[nH]c4)cnc32)CC1] |
| 79 | 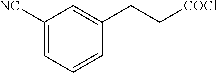 [SMILES: N#Cc1cccc(CCC(=O)Cl)c1] | 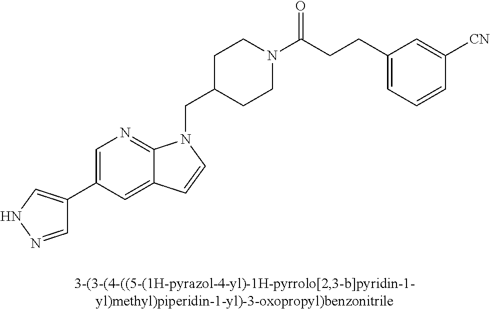 [SMILES: N#Cc1cccc(CCC(=O)N2CCC(Cn3ccc4cc(-c5cn[nH]c5)cnc43)CC2)c1] |

### Examples 80-85

Reaction of 1-((5,5-dimethylpyrrolidin-3-yl)methyl)-5-(1-(tetrahydro-2H-pyran-2-yl)-1H-pyrazol-4-yl)-1H-pyrrolo[2,3-b]pyri dine (Intermediate 58) and the acid chlorides shown in the table below using the general procedure described in Intermediate 16, followed by deprotection by treatment with methanolic p-toluene sulfonic acid at room temperature, the following can be prepared:

| | Acyl | |
|---|---|---|
| Example | Chloride | Product |
| 80 | 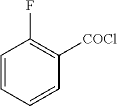 | 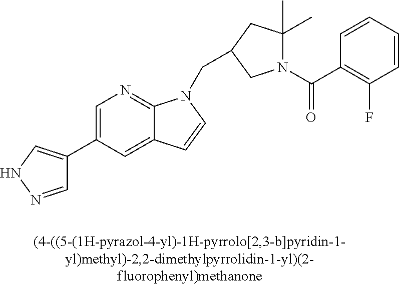 [SMILES: CC1(C)CC(Cn2ccc3cc(-c4cn[nH]c4)cnc32)CN1C(=O)c1ccccc1F] |
| 81 | 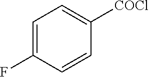 | 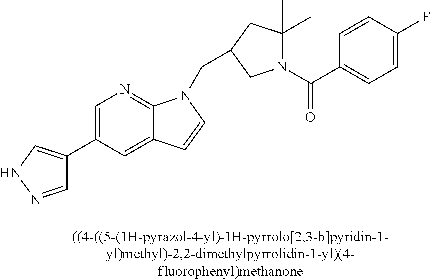 [SMILES: CC1(C)CC(Cn2ccc3cc(-c4cn[nH]c4)cnc32)CN1C(=O)c1ccc(F)cc1] |
| 82 | 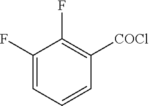 [SMILES: O=C(Cl)c1cccc(F)c1F] | 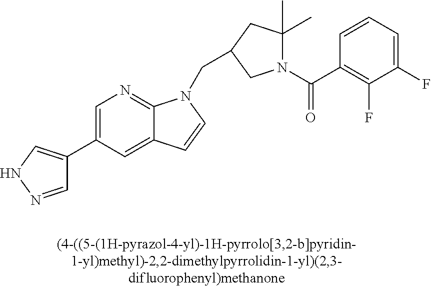 [SMILES: CC1(C)CC(Cn2ccc3cc(-c4cn[nH]c4)cnc32)CN1C(=O)c1cccc(F)c1F] |
| 83 | 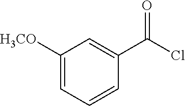 [SMILES: COc1cccc(C(=O)Cl)c1] | 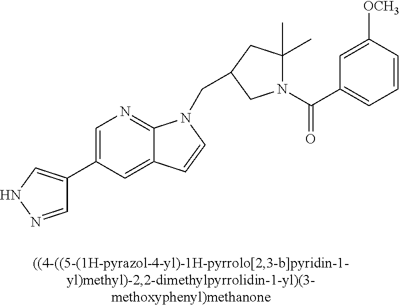 [SMILES: COc1cccc(C(=O)N2CC(Cn3ccc4cc(-c5cn[nH]c5)cnc43)CC2(C)C)c1] |
| 84 | 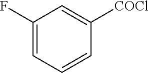 | 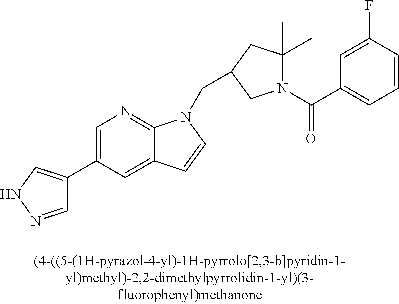 [SMILES: CC1(C)CC(Cn2ccc3cc(-c4cn[nH]c4)cnc32)CN1C(=O)c1cccc(F)c1] |
| 85 | 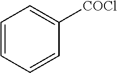 | 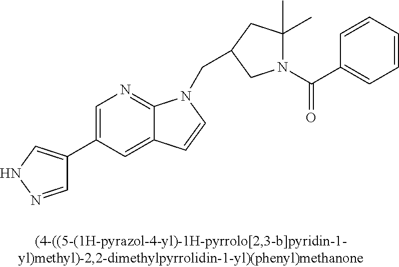 [SMILES: CC1(C)CC(Cn2ccc3cc(-c4cn[nH]c4)cnc32)CN1C(=O)c1ccccc1] |

### Examples 86-87

Reaction of 1-((5,5-dimethylpyrrolidin-3-yl)methyl)-5-(1-(tetrahydro-2H-pyran-2-yl)-1H-pyrazol-4-yl)-1H-pyrrolo[2,3-b]pyridine (Intermediate 58) and the acid chlorides shown in the table below using the general procedure described in Intermediate 16, followed by deprotection by treatment with methanolic p-toluene sulfonic acid at room temperature, the following can be prepared:

| Example | Acyl Chloride | Product |
|---|---|---|
| 86 | 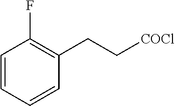 [SMILES: O=C(Cl)CCc1ccccc1F] | 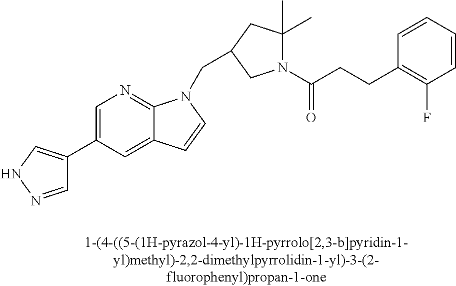 [SMILES: CC1(C)CC(Cn2ccc3cc(-c4cn[nH]c4)cnc32)CN1C(=O)CCc1ccccc1F] |
| 87 | 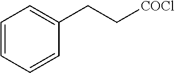 [SMILES: O=C(Cl)CCc1ccccc1] | 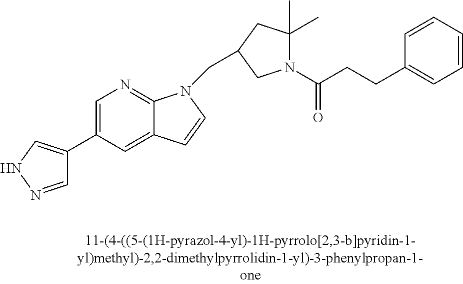 [SMILES: CC1(C)CC(Cn2ccc3cc(-c4cn[nH]c4)cnc32)CN1C(=O)CCc1ccccc1] |

### Examples 88-91

Reaction of rac.-cis-1-((5-methylpyrrolidin-3-yl)methyl)-5-(1-(tetrahydro-2H-pyran-2-yl)-1H-pyrazol-4-yl)-1H-pyrrolo[2,3-b]pyridine (Intermediate 59) and the acid chlorides shown in the table below using the general procedure described in Intermediate 16, followed by deprotection by treatment with methanolic p-toluene sulfonic acid at room temperature, the following can be prepared:

| | Acyl | |
|---|---|---|
| Example | Chloride | Product |
| 88 | 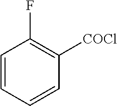 | 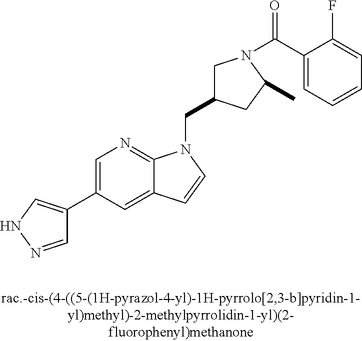 |
| 89 | 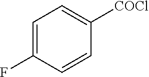 | 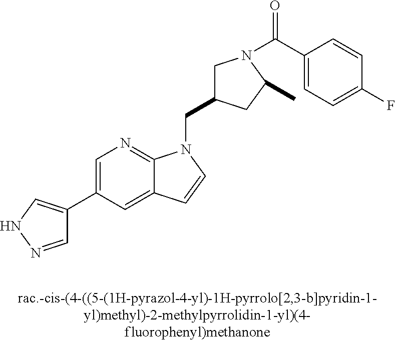 |
| 90 | 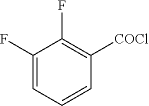 [SMILES: O=C(Cl)c1cccc(F)c1F] | 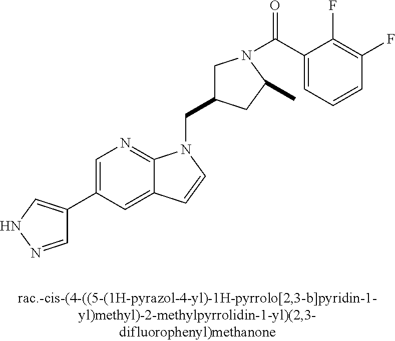 |
| 91 | 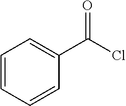 | 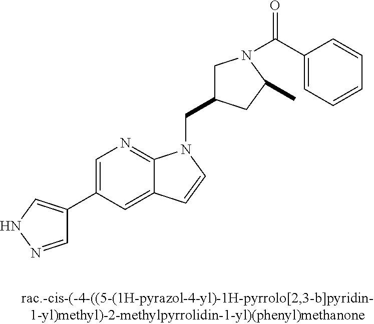 |

### Examples 92-96

Reaction of rac.-trans-1-((5-methylpyrrolidin-3-yl)methyl)-5-(1-(tetrahydro-2H-pyran-2-yl)-1H-pyrazol-4-yl)-1H-pyrrolo[2,3-b]pyridine (Intermediate 60) and the acid chlorides shown in the table below using the general procedure described in Intermediate 16, followed by deprotection by treatment with methanolic p-toluene sulfonic acid at room temperature, the following can be prepared:

| | Acyl | |
|---|---|---|
| Example | Chloride | Product |
| 92 | 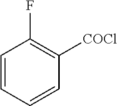 | 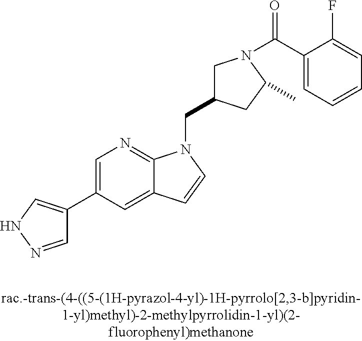 |
| 93 | 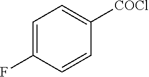 | 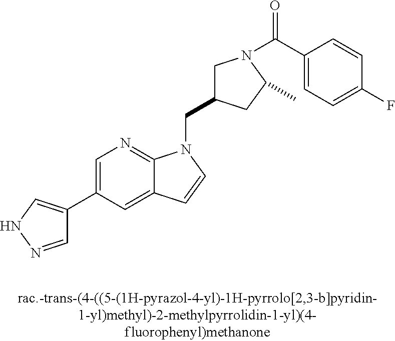 |
| 94 | 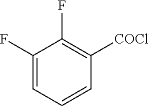 [SMILES: O=C(Cl)c1cccc(F)c1F] | 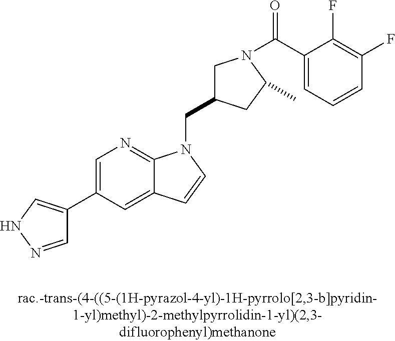 |
| 95 | 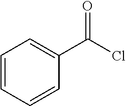 | 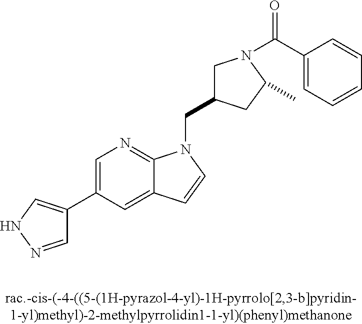 |

### Examples 96-97

Reaction of rac.-cis-1-((5-methylpyrrolidin-3-yl)methyl)-5-(1-(tetrahydro-2H-pyran-2-yl)-1H-pyrazol-4-yl)-1H-pyrrolo[2,3-b]pyridine (Intermediate 59) and the acid chlorides shown in the table below using the general procedure described in example 1, followed by deprotection by treatment with methanolic p-toluene sulfonic acid at room temperature, the following can be prepared:

| Example | Acyl Chloride | Product |
|---|---|---|
| 96 | 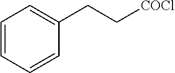 [SMILES: O=C(Cl)CCc1ccccc1] | 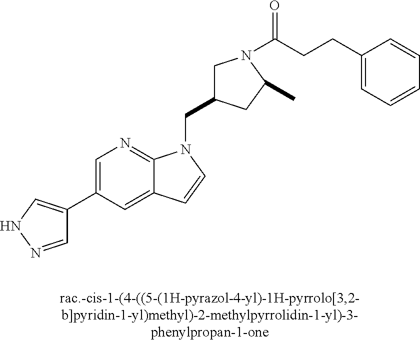 |
| 97 | 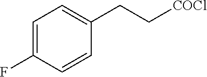 [SMILES: O=C(Cl)CCc1ccc(F)cc1] | 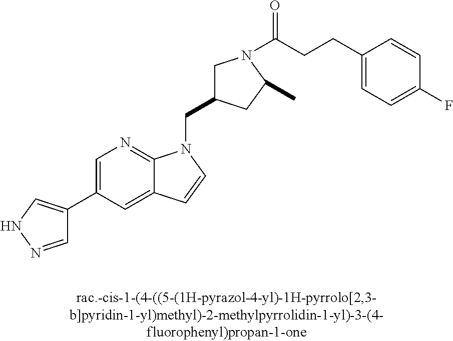 |

### Examples 98-99

Reaction of rac.-trans-1-((5-methylpyrrolidin-3-yl)methyl)-5-(1-(tetrahydro-2H-pyran-2-yl)-1H-pyrazol-4-yl)-1H-pyrrolo[2,3-b]pyridine (Intermediate 60) and the acid chlorides shown in the table below using the general procedure described in Intermediate 16, followed by deprotection by treatment with methanolic p-toluene sulfonic acid at room temperature, the following can be prepared:

| Example | Acyl Chloride | Product |
|---|---|---|
| 98 | 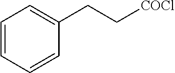 [SMILES: O=C(Cl)CCc1ccccc1] | 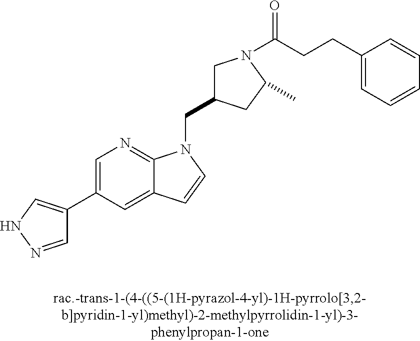 |
| 99 | 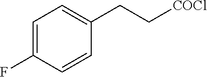 [SMILES: O=C(Cl)CCc1ccc(F)cc1] | 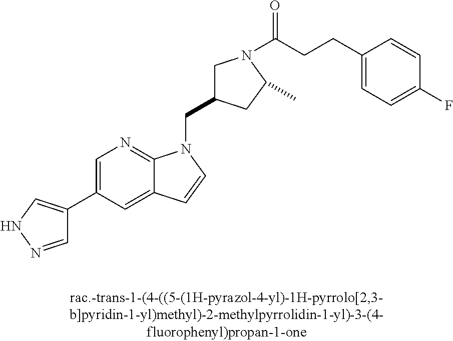 |

### Examples 100-102

By reaction of 1-((5-methylpyrrolidin-3-yl)methyl)-5-(1-(tetrahydro-2H-pyran-2-yl)-1H-pyrazol-4-yl)-1H-pyrrolo[2,3-b]pyridine (Intermediate 57) and the acid chlorides shown in the table below using the general procedure described in Intermediate 16, followed by deprotection by treatment with methanolic p-toluene sulfonic acid at room temperature, the following can be prepared:

| | Acyl | |
|---|---|---|
| Example | Chloride | Product |
| 100 | 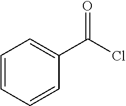 | 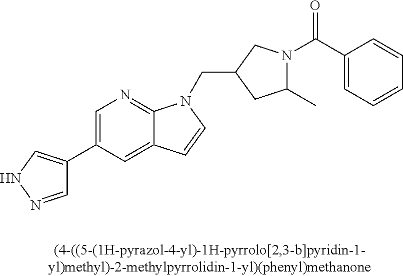 [SMILES: CC1CC(Cn2ccc3cc(-c4cn[nH]c4)cnc32)CN1C(=O)c1ccccc1] |
| 101 | 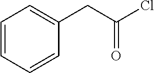 | 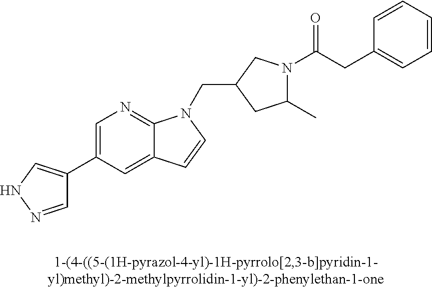 [SMILES: CC1CC(Cn2ccc3cc(-c4cn[nH]c4)cnc32)CN1C(=O)Cc1ccccc1] |
| 102 | 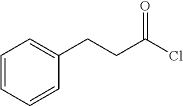 [SMILES: O=C(Cl)CCc1ccccc1] | 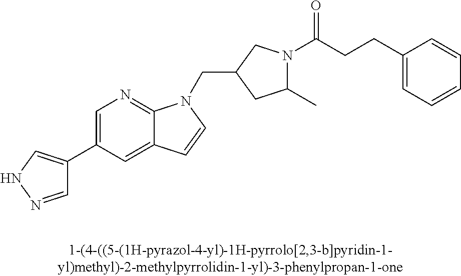 [SMILES: CC1CC(Cn2ccc3cc(-c4cn[nH]c4)cnc32)CN1C(=O)CCc1ccccc1] |

### Examples 103-104

By reaction of rac.-1-(azepan-4-ylmethyl)-5-(1-(tetrahydro-2H-pyran-2-yl)-1H-pyrazol-4-yl)-1H-pyrrolo[2,3-b]pyridine (Intermediate 61) and the acid chlorides shown in the table below using the general procedure described in Intermediate 16, followed by deprotection by treatment with methanolic p-toluene sulfonic acid at room temperature, the following can be prepared:

| | Acyl | |
|---|---|---|
| Example | Chloride | Product |
| 103 | 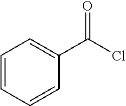 | 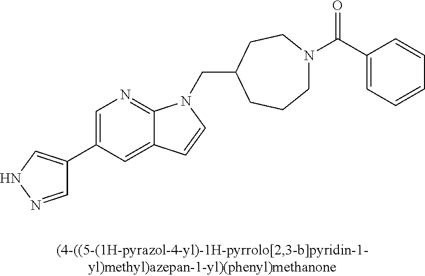 [SMILES: O=C(c1ccccc1)N1CCCC(Cn2ccc3cc(-c4cn[nH]c4)cnc32)CC1] |
| 104 | 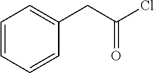 | 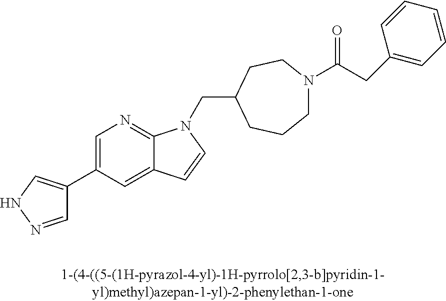 [SMILES: O=C(Cc1ccccc1)N1CCCC(Cn2ccc3cc(-c4cn[nH]c4)cnc32)CC1] |

### Examples 105-107

By reaction of rac.-benzyl 2-((5-(1-(tetrahydro-2H-pyran-2-yl)-1H-pyrazol-4-yl)-1H-pyrrolo[2,3-b]pyridin-1-yl)methyl)-7-azabicyclo[2.2.1]heptane-7-carboxylate (Intermediate 62) and the acid chlorides shown in the table below using the general procedure described in Intermediate 16, followed by deprotection by treatment with methanolic p-toluene sulfonic acid at room temperature, the following can be prepared:

| | Acyl | |
|---|---|---|
| Example | Chloride | Product |
| 105 | 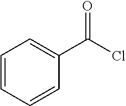 | 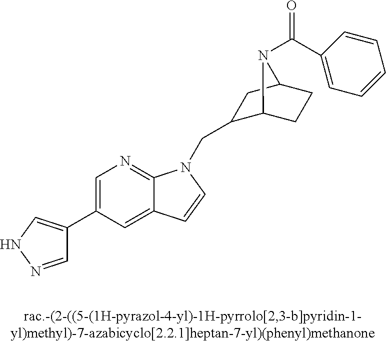 [SMILES: O=C(c1ccccc1)N1C2CCC1C(Cn1ccc3cc(-c4cn[nH]c4)cnc31)C2] |
| 106 | 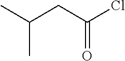 | 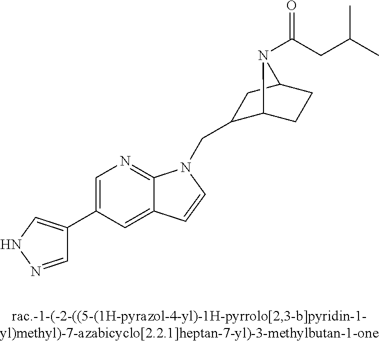 [SMILES: CC(C)CC(=O)N1C2CCC1C(Cn1ccc3cc(-c4cn[nH]c4)cnc31)C2] |
| 107 | 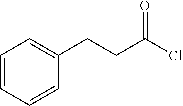 [SMILES: O=C(Cl)CCc1ccccc1] | 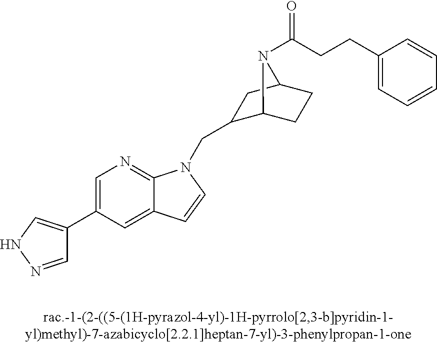 [SMILES: O=C(CCc1ccccc1)N1C2CCC1C(Cn1ccc3cc(-c4cn[nH]c4)cnc31)C2] |

### Example 108

### Synthesis of 1-(4-((5-(5-methyl-1H-pyrazol-4-yl)-1H-pyrrolo[2,3-c]pyridin-1-yl)methyl)piperidin-1-yl)-3-phenylpropan-1-one

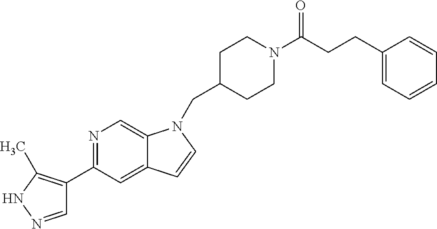 [SMILES: Cc1[nH]ncc1-c1cc2ccn(CC3CCN(C(=O)CCc4ccccc4)CC3)c2cn1]

By reaction of 1-(4-((5-bromo-1H-pyrrolo[2,3-c]pyridin-1-yl)methyl)piperidin-1-yl)-3-phenylpropan-1-one (Intermediate 64) and 4-bromo-1-(tetrahydro-2H-pyran-2-yl)-5-(trifluoromethyl)-1H-pyrazole (Intermediate 24) using the general procedure described in example 1, followed by deprotection by treatment with methanolic p-toluene sulfonic acid at room temperature, 1-(4-((5-(5-methyl-1H-pyrazol-4-yl)-1H-pyrrolo[2,3-b]pyridin-1-yl)methyl)piperidin-1-yl)-3-phenylpropan-1-one can be prepared.

### Example 109

### Synthesis of 1-(4-((5-(5-methyl-1H-pyrazol-4-yl)-1H-pyrrolo [2,3-b]pyridin-1-yl)methyl)piperidin-1-yl)-3-phenylpropan-1-one

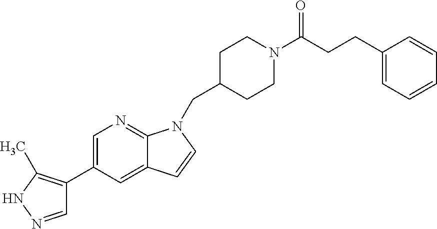 [SMILES: Cc1[nH]ncc1-c1cnc2c(ccn2CC2CCN(C(=O)CCc3ccccc3)CC2)c1]

By reaction of 1-(4-((5-bromo-1H-pyrrolo[2,3-b]pyridin-1-yl)methyl)piperidin-1-yl)-3-phenylpropan-1-one (Intermediate 64) and 4-bromo-1-(tetrahydro-2H-pyran-2-yl)-5-(trifluoromethyl)-1H-pyrazole (Intermediate 24) using the general procedure described in example 1, followed by deprotection by treatment with methanolic p-toluene sulfonic acid at room temperature, 1-(4-((5-(5-methyl-1H-pyrazol-4-yl)-1H-pyrrolo[2,3-b]pyridin-1-yl)methyl)piperidin-1-yl)-3-phenylpropan-1-one can be prepared.

### Examples 110-111

By reaction of (4-((5-bromo-1H-pyrrolo[2,3-c]pyridin-1-yl)methyl)piperidin-1-yl)(2-fluorophenyl)methanone (Intermediate 9) with the heteroaryl bromides shown below under the general conditions of Example 1, followed by deprotection by treatment with methanolic p-toluene sulfonic acid, the following can be prepared:

| | Acyl | |
|---|---|---|
| Example | Chloride | Product |
| 110 | 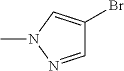 | 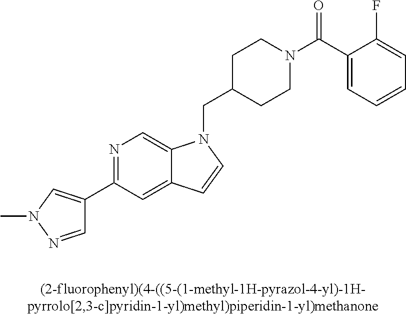 [SMILES: Cn1cc(-c2cc3ccn(CC4CCN(C(=O)c5ccccc5F)CC4)c3cn2)cn1] |
| 111 | 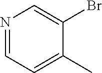 | 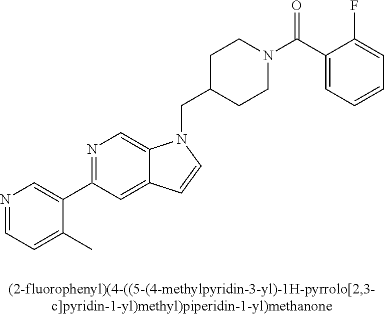 [SMILES: Cc1ccncc1-c1cc2ccn(CC3CCN(C(=O)c4ccccc4F)CC3)c2cn1] |

### Example 112

### Synthesis of (4-((5-(1H-pyrazol-4-yl)-1H-pyrrolo[2,3-c]pyridin-1-yl)methyl)-4-fluoropiperidin-1-yl)(2-fluorophenyl)methanone

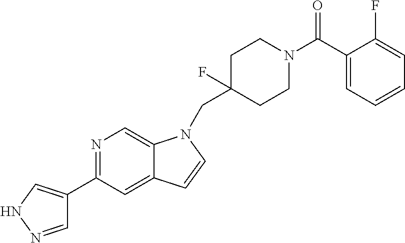 [SMILES: O=C(c1ccccc1F)N1CCC(F)(Cn2ccc3cc(-c4cn[nH]c4)ncc32)CC1]

(4-((5-(1H-Pyrazol-4-yl)-1H-pyrrolo[2,3-c]pyridin-1-yl)methyl)-4-fluoropiperidin-1-yl)(2-fluorophenyl)methanone can be prepared by reaction of (2-fluorophenyl)(1-oxa-6-azaspiro[2.5]octan-6-yl)methanone with 5-bromo-1H-pyrrolo[2,3-c]pyridine (Intermediate 3) in the presence of NaH in DMF followed by treatment with DAST and coupling with 1-(tetrahydro-2H-pyran-2-yl)-4-(4,4,5,5-tetramethyl-1,3,2-dioxaborolan-2-yl)-1H-pyrazole under the conditions employed in example 1 followed by deprotection with methanolic p-toluene sulfonic acid.

### Examples 113-118

By condensation of 1-(piperidin-4-ylmethyl)-5-(1-(tetrahydro-2H-pyran-2-yl)-1H-pyrazol-4-yl)-1H-pyrrolo[2,3-c]pyridine (Intermediate 31) with the carboxylic acids shown below using peptide coupling conditions promoted by HATU, followed by deprotection using methanolic p-toluene sulfonic acid, the following can be prepared:

| Example | Acyl Chloride | Product |
|---|---|---|
| 113 | 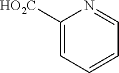 | 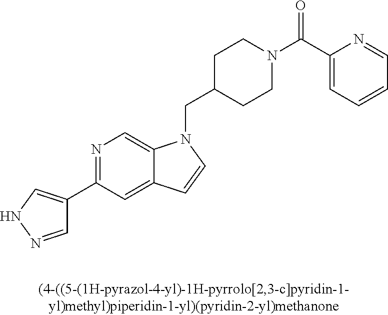 [SMILES: O=C(c1ccccn1)N1CCC(Cn2ccc3cc(-c4cn[nH]c4)ncc32)CC1] |
| 114 | 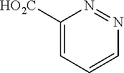 | 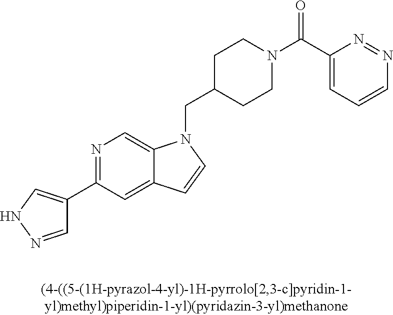 [SMILES: O=C(c1cccnn1)N1CCC(Cn2ccc3cc(-c4cn[nH]c4)ncc32)CC1] |
| 115 | 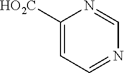 | 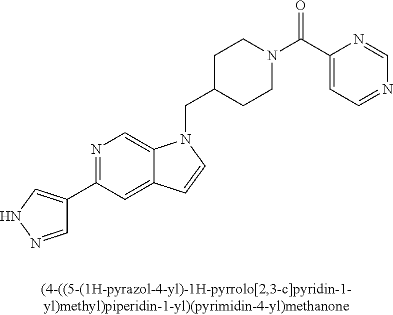 [SMILES: O=C(c1ccncn1)N1CCC(Cn2ccc3cc(-c4cn[nH]c4)ncc32)CC1] |
| 116 | 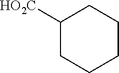 | 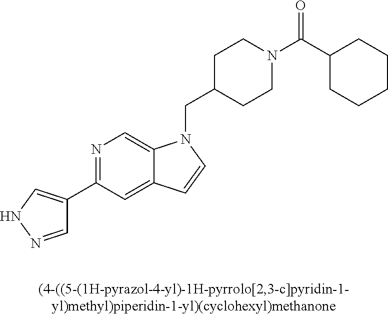 [SMILES: O=C(C1CCCCC1)N1CCC(Cn2ccc3cc(-c4cn[nH]c4)ncc32)CC1] |
| 117 | 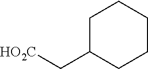 | 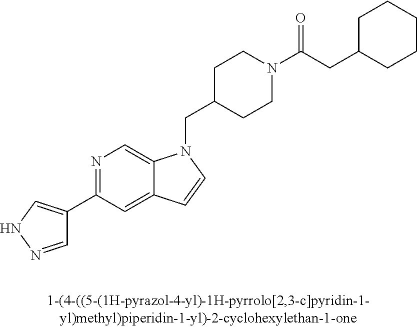 [SMILES: O=C(CC1CCCCC1)N1CCC(Cn2ccc3cc(-c4cn[nH]c4)ncc32)CC1] |
| 118 | 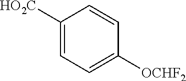 [SMILES: O=C(O)c1ccc(OC(F)F)cc1] | 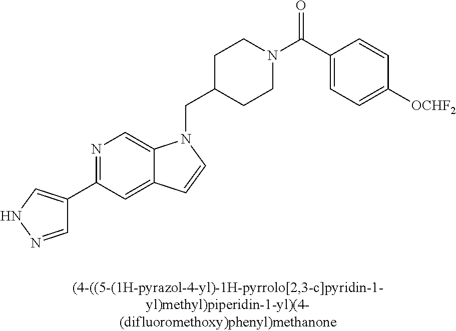 [SMILES: O=C(c1ccc(OC(F)F)cc1)N1CCC(Cn2ccc3cc(-c4cn[nH]c4)ncc32)CC1] |

### Example 119

### Preparation of 1-(4-((5-(1H-pyrazol-4-yl)-1H-pyrrolo [2,3-c]pyridin-1-yl)methyl)piperidin-1-yl)-4,4,4-trifluoro-3-hydroxy-3-(trifluoromethyl)butan-1-one

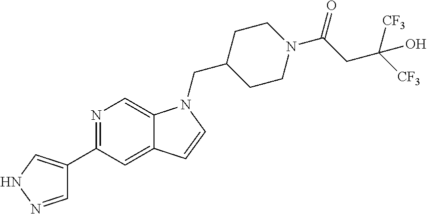 [SMILES: O=C(CC(O)(C(F)(F)F)C(F)(F)F)N1CCC(Cn2ccc3cc(-c4cn[nH]c4)ncc32)CC1]

1-(4-((5-(1H-pyrazol-4-yl)-1H-pyrrolo[2,3-c]pyridin-1-yl)methyl)piperidin-1-yl)-4,4,4-trifluoro-3-hydroxy-3-(trifluoromethyl)butan-1-one can be prepared in two steps by coupling of 1-(piperidin-4-ylmethyl)-5-(1-(tetrahydro-2H-pyran-2-yl)-1H-pyrazol-4-yl)-1H-pyrrolo[2,3-c]pyridine (Intermediate 31) and 4,4,4-trifluoro-3-hydroxy-3-(trifluoromethyl)butanoic acid mediated by BOP and triethylamine in dichloromethane followed by deprotection using the conditions employed in example 5.

### Examples 120-122

Reaction of 1-(piperidin-4-ylmethyl)-5-(1-(tetrahydro-2H-pyran-2-yl)-1H-pyrazol-4-yl)-1H-pyrrolo[2,3-c]pyridine (Intermediate 31) and the acid chlorides shown in the table below using the general procedure described in Intermediate 6, followed by deprotection by treatment with methanolic p-toluene sulfonic acid at room temperature, the following can be prepared:

| | Acyl | |
|---|---|---|
| Example | Chloride | Product |
| 120 | 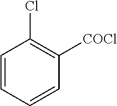 | 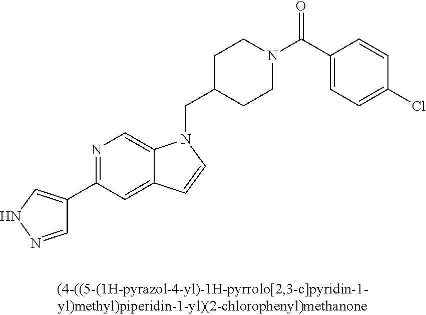 [SMILES: O=C(c1ccc(Cl)cc1)N1CCC(Cn2ccc3cc(-c4cn[nH]c4)ncc32)CC1] |
| 121 | 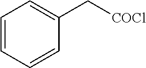 | 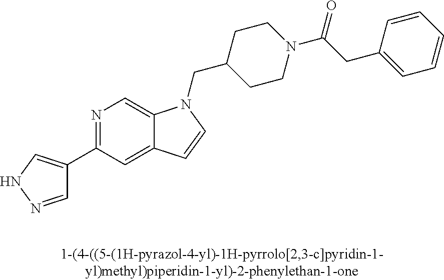 [SMILES: O=C(Cc1ccccc1)N1CCC(Cn2ccc3cc(-c4cn[nH]c4)ncc32)CC1] |
| 122 | 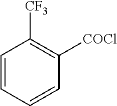 [SMILES: O=C(Cl)c1ccccc1C(F)(F)F] | 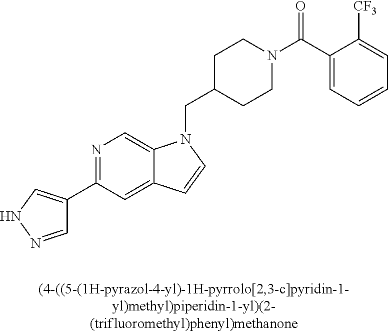 [SMILES: O=C(c1ccccc1C(F)(F)F)N1CCC(Cn2ccc3cc(-c4cn[nH]c4)ncc32)CC1] |

### Example 123

### Human T_{H}17 Cytokine Inhibition as Measured by ELISA

Peripheral blood mononuclear cells (PBMCs) were sourced from freshly prepared leukocyte enriched plasma (buffy coat) from healthy donors (New York Blood Center). PBMCs were isolated by density gradient centrifugation using Ficoll-Paque™ PLUS (GE Healthcare). Human CD4+ T cells were seeded into 96-well plates (5×10⁴ cells/well) and activated with plate-bound anti-human (h)-CD3 antibody and soluble h-aCD28 (both at 1 ug/ml; eBioscience) and differentiated into T_{H}17 cells with 20 ng/mL h-IL-6, 5 ng/mL h-TGF-β1, 10 ng/mL h-IL-23 (eBioscience) and 10 ng/mL IL-1β (Miltenyi Biotec) in serum-free TexMACS Medium (Miltenyi Biotec) supplemented with 1% Penicillin/Streptomycin (Lonza) for 3 days. CD4+ T cells propagated under T_{H}17-polarizing conditions were cultured in the presence or absence of various concentrations of compounds with a final concentration of 0.1% DMSO. Supernatants were collected and stored at −20° C. until assayed for IL-17A and IL-17F levels by “Ready-Set-Go” ELISA kits (eBioscience) as per manufacturer's instructions. Endpoint absorbance was read at 450 nm using a microplate reader (Perkin Elmer). The half maximal inhibitory concentrations (IC₅₀) for representative compounds of the invention were determined by GraphPad Prism® software and presented in the table below:

| Example | IL-17A | IL-17F |
|---|---|---|
| Number | IC₅₀ μM | IC₅₀ μM |
| 1 | <10 | <10 |
| 2 | <10 | <10 |
| 3 | <10 | <10 |
| 4 | <10 | <10 |
| 5 | <10 | <10 |
| 6 | <10 | <10 |
| 7 | <10 | <10 |
| 8 | <10 | <10 |
| 9 | <10 | <10 |
| 10 | <10 | <10 |
| 11 | <10 | <10 |
| 12 | <10 | <10 |
| 13 | <10 | <10 |
| 14 | <10 | <10 |
| 15 | <10 | <10 |
| 16 | <10 | <10 |

The invention is further described in the following numbered paragraphs:

1. A compound of formula (I):

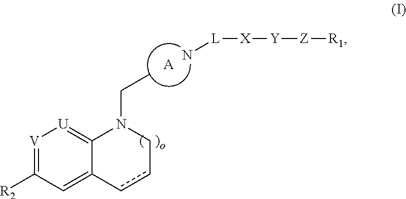

wherein:
A is a mono- or bicyclic 5- to 8-membered heterocyclic ring having one ring carbon replaced by N as shown, said ring optionally mono- or bi-substituted on one or more ring carbons independently with a C₁-C₆ alkyl group, or halogen;

### L is —C(O)— or —S(O)₂;

X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
One of U and V is N and the other is CH;
R₁ is —C₁-C₆ alkyl, optionally mono- or bi-substituted with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
   R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O); [symbol]
   is a single or double bond;
   o is 0 or 1;
   n is 0 or 1;
   p is 0, 1 or 2; and
   q is 0 or 1,
   or a pharmaceutically acceptable salt thereof.

2. The compound according to paragraph 1, wherein A is unsubstituted piperidinyl, pyrrolidinyl, azepanyl or azabicyclo[2.2.1]heptanyl.

3. The compound according to paragraph 1, wherein A is piperidinyl, pyrrolidinyl, azabicyclo[2.2.1]heptanyl or azepanyl mono- or bi-substituted independently with C₁-C₆ alkyl or halogen.

4. The compound according to paragraph 1, wherein A is piperidinyl, pyrrolidinyl, azepanyl or azabicyclo[2.2.1]heptanyl mono-substituted with methyl or fluorine.

5. The compound according to paragraph 1, wherein A is piperidinyl, pyrrolidinyl or azepanyl bi-substituted with methyl or fluorine.

6. The compound according to paragraph 1, wherein X is —CH₂—, —O— or —NH—.

7. The compound according to paragraph 1, wherein Y is —O—.

8. The compound according to paragraph 1, wherein R₁ is —C₁-C₆ alkyl, optionally substituted independently with perfluoroalkyl or —OH.

9. The compound according to paragraph 1, wherein R₁ is methyl, ethyl, propyl or t-butyl.

10. The compound according to paragraph 1, wherein R₁ is unsubstituted phenyl.

11. The compound according to paragraph 1, wherein R₁ is phenyl substituted with halogen, alkoxy or C₁-C₆ alkyl.

12. The compound according to paragraph 1, wherein R₁ is chlorophenyl, dichlorophenyl, fluorophenyl or difluorophenyl.

13. The compound according to paragraph 1, wherein R₁ is methoxy-phenyl.

14. The compound according to paragraph 1, wherein R₁ is methyl-phenyl.

15. The compound according to paragraph 1, wherein R₁ is cycloalkyl.

16. The compound according to paragraph 1, wherein R₁ is cyclopentyl or cyclohexyl.

17. The compound according to paragraph 1, wherein R₁ is an unsubstituted 5- or 6-membered heteroaryl group having one or more ring carbons replaced by N

18. The compound according to paragraph 1, wherein R₁ is a 5- or 6-membered heteroaryl group, having one or more ring carbons replaced by N, optionally substituted with a C₁-C₆ alkyl group.

19. The compound according to paragraph 1, wherein R₂ is an unsubstituted 5- to 7-membered heteroaryl group having one, two or three ring carbons replaced by N.

20. The compound according to paragraph 1, wherein R₂ is a 5- to 7-membered heteroaryl group having one, two or three ring carbons replaced by N, mono- or bi-substituted independently with C₁-C₆ alkyl, —CN or (═O).

21. The compound according to paragraph 1, wherein R₂ is unsubstituted pyrazolyl, pyridinyl or triazolyl.

22. The compound according to paragraph 1, wherein R₂ is pyrazolyl, pyridinyl or triazolyl, mono- or bi-substituted independently with methyl, nitrile or CF₃.

23. The compound according to paragraph 1, wherein R₂ is unsubstituted pyrazolyl.

24. The compound according to paragraph 1, wherein R₂ is linked via a carbon atom.

25. The compound according to paragraph 1, having a formula (Ia):

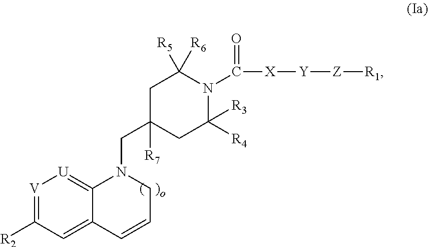

wherein:
X is —(CH₂)ₙ—, —O— or —NH—.
Y is —(CH₂)ₚ—, —O—, —S— or —SO₂—, with the proviso that X and Y are not both a heteroatom;
Z is —(CH₂)_{q}—;
One of U and V is N and the other is CH;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl or
a 5- or 6-membered heteroaryl group having one or more ring carbons independently replaced by N, O or S, said heteroaryl optionally mono- or bi-substituted independently with halogen, alkoxy, C₁-C₆ alkyl, —CN, or perfluorinated C₁-C₆ alkyl;
   R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O);
   R₃, R₄, R₅ and R₆ are, independently of each other, H or —C₁-C₆ alkyl;

### R₇ is H or F;

[symbol] is a single or double bond;
o is 0 or 1;
n is 0 or 1;
p is 0, 1 or 2; and
q is 0 or 1,
or a pharmaceutically acceptable salt thereof.

26. The compound according to paragraph 1, having a formula (Ib):

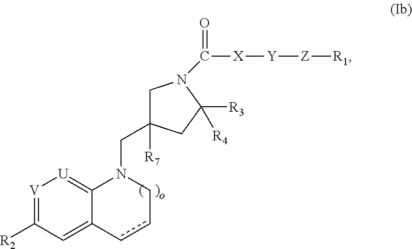

wherein:
X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
One of U and V is N and the other is CH;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
   R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O);
   R₃ and R₄ are, independently of each other, H or —C₁-C₆ alkyl;

### R₇ is H or F;

[symbol] is a single or double bond;
o is 0 or 1;
n is 0 or 1;
p is 0, 1 or 2; and
q is 0 or 1,
or a pharmaceutically acceptable salt thereof.

27. The compound according to paragraph 1, having a formula (Ic):

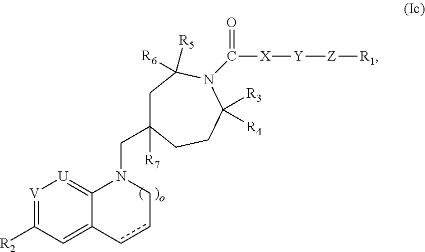

wherein:
X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
One of U and V is N and the other is CH;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
   R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O);
   R₃, R₄ R₅, and R₆ are, independently of each other, H or —C₁-C₆ alkyl;

### R₇ is H or F;

[symbol] is a single or double bond;
o is 0 or 1;
n is 0 or 1;
p is 0, 1 or 2; and
q is 0 or 1,
or a pharmaceutically acceptable salt thereof.

28. The compound according to paragraph 1, having a formula (Id):

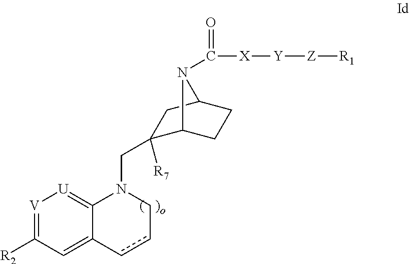

wherein:
X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
One of U and V is N and the other is CH;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
   R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O);

### R₇ is H or F;

[symbol] is a single or double bond;
o is 0 or 1;
n is 0 or 1;
p is 0, 1 or 2; and
q is 0 or 1,
or a pharmaceutically acceptable salt thereof.

29. The compound according to paragraph 1, having a formula (Ie):

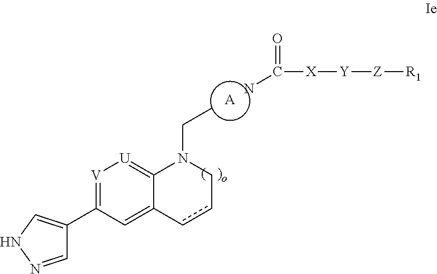

wherein:
A is a monocyclic or bicyclic 5- to 8-membered heterocyclic ring having one ring carbon replaced by N as shown, said ring optionally mono- or bi-substituted on one or more ring carbons independently with a C₁-C₆ alkyl group, or halogen;
X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
One of U and V is N and the other is CH;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
   R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O); [symbol]
   is a single or double bond;
   o is 0 or 1;
   n is 0 or 1;
   p is 0, 1 or 2; and
   q is 0 or 1,
   or a pharmaceutically acceptable salt thereof.

30. The compound according to paragraph 1, having a formula (If):

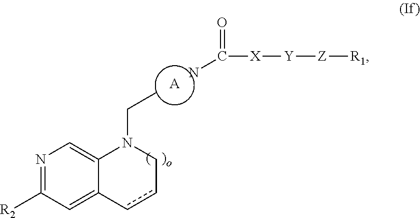

wherein:
A is a monocyclic or bicyclic 5- to 8-membered heterocyclic ring having one ring carbon replaced by N as shown, said ring optionally mono- or bi-substituted on one or more ring carbons independently with a C₁-C₆ alkyl group, or halogen;
X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
   R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O);
   o is 0 or 1;
   n is 0 or 1;
   p is 0, 1 or 2; and
   q is 0 or 1,
   or a pharmaceutically acceptable salt thereof.

31. The compound according to paragraph 1, having a formula (Ig):

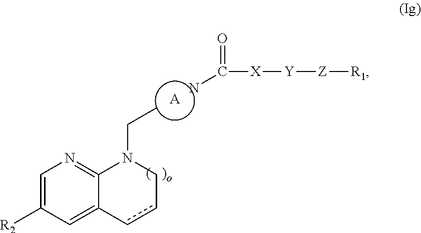

wherein:
A is a monocyclic or bicyclic 5- to 8-membered heterocyclic ring having one ring carbon replaced by N as shown, said ring optionally mono- or bi-substituted on one or more ring carbons independently with a C₁-C₆ alkyl group, or halogen;
X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
   R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O); [symbol]
   is a single or double bond;
   o is 0 or 1;
   n is 0 or 1;
   p is 0, 1 or 2; and
   q is 0 or 1,
   or a pharmaceutically acceptable salt thereof.

32. The compound according to paragraph 1, having a formula I(h):

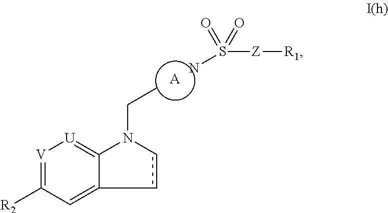

wherein:
A is a monocyclic or bicyclic 5- to 8-membered heterocyclic ring having one ring carbon replaced by N as shown, said ring optionally mono- or bi-substituted on one or more ring carbons independently with a C₁-C₆ alkyl group, or halogen; One of U and V is N and the other is CH;
Z is —(CH₂)_{q}— or abent;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, alkylsulfonyloxy, alkylsulfonyl, halo-C₁-C₆ alkyl or C₁-C₅ cycloalkyl,
cycloalkyl, optionally mono- or bi-substituted independently with halogen, alkoxy, C₁-C₆ alkyl, —CN, alkylsulfonyloxy, alkylsulfonyl, halo-C₁-C₆ alkyl or C₁-C₅ cycloalkyl,
heterocycle, optionally mono- or bi-substituted independently with halogen, alkoxy, C₁-C₆ alkyl, —CN, alkylsulfonyloxy, alkylsulfonyl, halo-C₁-C₆ alkyl or C₁-C₅ cycloalkyl or
a 5- or 6-membered heteroaryl group having one or more ring carbons independently replaced by N, O or S, said heteroaryl optionally substituted with halogen, alkoxy, C₁-C₆ alkyl, —CN, or perfluorinated C₁-C₆ alkyl;
   R₂ is a 5- to 7-membered heteroaryl group having one, two or three ring carbons independently replaced by N, O or S, said heteroaryl optionally mono- or bi-substituted independently with C₁-C₆ alkyl, —CN or (═O); [symbol]
   is a single or double bond; and
   q is 0, 1 or 2.
   or a pharmaceutically acceptable salt thereof.

33. The compound of paragraph 32, wherein U is N and V is CH.

34. The compound of paragraph 32, wherein U is CH and V is N.

35. The compound of paragraph 32, wherein A is unsubstituted piperidinyl, pyrrolidinyl, bicyclo[2,2,1]azepinyl or azepanyl.

36. The compound of paragraph 32, wherein A is piperidinyl, pyrrolidinyl, azepanyl or bicyclo[2,2,1]azepinyl mono- or bi-substituted independently with C₁-C₆ alkyl or halogen.

37. The compound of paragraph 32, wherein A is piperidinyl, pyrrolidinyl, azepanyl or bicyclo[2,2,1]azepinyl mono-substituted with methyl or fluorine.

38. The compound of paragraph 32, wherein A is piperidinyl, pyrrolidinyl, azepanyl or bicyclo[2,2,1]azepinyl bi-substituted independently with methyl or fluorine.

39. The compound of paragraph 32, wherein R₁ is —C₁-C₆ alkyl optionally substituted with perfluoroalkyl.

40. The compound of paragraph 32, wherein R₁ is methyl, ethyl, propyl or t-butyl.

41. The compound of paragraph 32, wherein R₁ is unsubstituted phenyl.

42. The compound of paragraph 32, wherein R₁ is phenyl substituted with halogen, alkylsulfonyl, alkoxy, —CN, alkyl, or C₁-C₆ alkyl.

43. The compound of paragraph 32, wherein R₁ is cycloalkyl.

44. The compound of paragraph 32, wherein R₁ is an unsubstituted 5- or 6-membered heteroaryl group having one or more ring carbons replaced by N.

45. The compound of paragraph 32, wherein R₂ is an unsubstituted 5- to 7-membered heteroaryl group having one, two or three ring carbons replaced by N.

46. The compound of paragraph 32, wherein R₂ is unsubstituted pyrazolyl or triazolyl.

47. The compound of paragraph 32, wherein R₂ is unsubstituted pyrazolyl.

48. The compound of paragraph 32, wherein R₂ is linked via a carbon atom.

49. The compound according to paragraph 1, having a formula I(i):

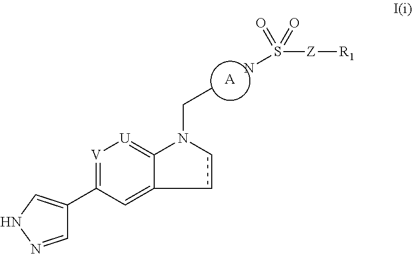

wherein:
A is a monocyclic or bicyclic 5- to 8-membered heterocyclic ring having one ring carbon replaced by N as shown, said ring optionally mono- or bi-substituted on one or more ring carbons independently with a C₁-C₆ alkyl group, or halogen;
One of U and V is N and the other is CH;
Z is —(CH₂)_{q}— or absent;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, alkylsulfonyloxy, alkylsulfonyl, halo-C₁-C₆ alkyl or C₁-C₅ cycloalkyl,
cycloalkyl, optionally mono- or bi-substituted independently with halogen, alkoxy, C₁-C₆ alkyl, —CN, alkylsulfonyloxy, alkylsulfonyl, halo-C₁-C₆ alkyl or C₁-C₅ cycloalkyl,
heterocycle, optionally mono- or bi-substituted independently with halogen, alkoxy, C₁-C₆ alkyl, —CN, alkylsulfonyloxy, alkylsulfonyl, halo-C₁-C₆ alkyl or C₁-C₅ cycloalkyl or
a 5- or 6-membered heteroaryl group having one or more ring carbons independently replaced by N, O or S, said heteroaryl optionally mono- or bi-substituted independently with halogen, alkoxy, C₁-C₆ alkyl, —CN, or perfluorinated C₁-C₆ alkyl; [symbol]
   is a single or double bond; and
   q is 0, 1 or 2.
   or a pharmaceutically acceptable salt thereof.

50. The compound according to paragraph 1, wherein said compound is:
(4-((5-(1H-pyrazol-4-yl)-1H-pyrrolo[2,3-c]pyridin-1-yl)methyl)piperidin-1-yl)(phenyl)methanone;
1-(4-((5-(1H-pyrazol-4-yl)-1H-pyrrolo[2,3-c]pyridin-1-yl)methyl)piperidin-1-yl)-3-phenylpropan-1-one;
(4-((5-(1H-pyrazol-4-yl)-1H-pyrrolo[2,3-c]pyridin-1-yl)methyl)piperidin-1-yl)(phenyl)methanone;
(4-((5-(1H-pyrazol-4-yl)-1H-pyrrolo[2,3-c]pyridin-1-yl)methyl)piperidin-1-yl)(2-fluorophenyl)methanone;
(4-((5-(1H-pyrazol-4-yl)-1H-pyrrolo[2,3-c]pyridin-1-yl)methyl)piperidin-1-yl)(2-fluorophenyl)methanone;
(4-((5-(1H-pyrazol-4-yl)-1H-pyrrolo[2,3-c]pyridin-1-yl)methyl)piperidin-1-yl)(2-fluorophenyl)methanone;
1-((1-(phenethyl sulfonyl)piperidin-4-yl)methyl)-5-(1H-pyrazol-4-yl)-1H-pyrrolo[2,3-c]pyri dine;
1-((1-(isobutylsulfonyl)piperidin-4-yl)methyl)-5-(1H-pyrazol-4-yl)-1H-pyrrolo[2,3-c]pyridine;
1-((1-(phenylsulfonyl)piperidin-4-yl)methyl)-5-(1H-pyrazol-4-yl)-1H-pyrrolo[2,3-c]pyridine;
1-((1-((2-fluorophenyl)sulfonyl)piperidin-4-yl)methyl)-5-(1H-pyrazol-4-yl)-1H-pyrrolo[2,3-c]pyridine;
(4-((5-(1H-pyrazol-4-yl)-1H-pyrrolo[2,3-b]pyridin-1-yl)methyl)piperidin-1-yl)(phenyl)methanone;
1-(4-((5-(1H-pyrazol-4-yl)-1H-pyrrolo[2,3-b]pyridin-1-yl)methyl)piperidin-1-yl)-3-phenylpropan-1-one;
1-(4-((5-(1H-pyrazol-4-yl)-1H-pyrrolo[2,3-b]pyridin-1-yl)methyl)piperidin-1-yl)-3-methylbutan-1-one;
(4-((5-(1H-pyrazol-4-yl)-1H-pyrrolo[2,3-b]pyridin-1-yl)methyl)piperidin-1-yl)(2-fluorophenyl)methanone;
1-((1-(phenethylsulfonyl)piperidin-4-yl)methyl)-5-(1H-pyrazol-4-yl)-1H-pyrrolo[2,3-b]pyridine;
1-((1-(isobutylsulfonyl)piperidin-4-yl)methyl)-5-(1H-pyrazol-4-yl)-1H-pyrrolo[2,3-b]pyridine;
1-((1-(phenylsulfonyl)piperidin-4-yl)methyl)-5-(1H-pyrazol-4-yl)-1H-pyrrolo[2,3-b]pyridine;
1-((1-((2-fluorophenyl)sulfonyl)piperidin-4-yl)methyl)-5-(1H-pyrazol-4-yl)-1H-pyrrolo[2,3-b]pyridine;
(4-((5-(1H-pyrazol-4-yl)-1H-pyrrolo[2,3-c]pyridin-1-yl)methyl)piperidin-1-yl)(2-(trifluoromethyl)phenyl)methanone;
(4-((5-(1H-pyrazol-4-yl)-1H-pyrrolo[2,3-c]pyridin-1-yl)methyl)piperidin-1-yl)(4-fluorophenyl)methanone;
(4-((5-(1H-pyrazol-4-yl)-1H-pyrrolo[2,3-c]pyridin-1-yl)methyl)piperidin-1-yl)(2,3-difluorophenyl)methanone;
(4-((5-(1H-pyrazol-4-yl)-1H-pyrrolo[2,3-c]pyridin-1-yl)methyl)piperidin-1-yl)(2,4-difluorophenyl)methanone;
(4-((5-(1H-pyrazol-4-yl)-1H-pyrrolo[2,3-c]pyridin-1-yl)methyl)piperidin-1-yl)(p-tolyl)methanone;
4-(4-((5-(1H-pyrazol-4-yl)-1H-pyrrolo[2,3-c]pyridin-1-yl)methyl)piperidine-1-carbonyl)benzonitrile;
(4-((5-(1H-pyrazol-4-yl)-1H-pyrrolo[2,3-c]pyridin-1-yl)methyl)piperidin-1-yl)(3-fluorophenyl)methanone;
3-(4-((5-(1H-pyrazol-4-yl)-1H-pyrrolo[2,3-c]pyridin-1-yl)methyl)piperidine-1-carbonyl)benzonitrile;
(4-((5-(1H-pyrazol-4-yl)-1H-pyrrolo[2,3-c]pyridin-1-yl)methyl)piperidin-1-yl)(2,6-difluorophenyl)methanone;
1-(4-((5-(1H-pyrazol-4-yl)-1H-pyrrolo[2,3-c]pyridin-1-yl)methyl)piperidin-1-yl)-3-(2-fluorophenyl)propan-1-one;
11-(4-((5-(1H-pyrazol-4-yl)-1H-pyrrolo[2,3-c]pyridin-1-yl)methyl)piperidin-1-yl)-3-(4-fluorophenyl)propan-1-one;
1-(4-((5-(1H-pyrazol-4-yl)-1H-pyrrolo[2,3-c]pyridin-1-yl)methyl)piperidin-1-yl)-3-(2,3-difluorophenyl)propan-1-one;
1-(4-((5-(1H-pyrazol-4-yl)-1H-pyrrolo[2,3-c]pyridin-1-yl)methyl)piperidin-1-yl)-3-(2,4-difluorophenyl)propan-1-one;
1-(4-((5-(1H-pyrazol-4-yl)-1H-pyrrolo[2,3-c]pyridin-1-yl)methyl)piperidin-1-yl)-3-(p-tolyl)propan-1-one;
4-(3-(4-((5-(1H-pyrazol-4-yl)-1H-pyrrolo[2,3-c]pyridin-1-yl)methyl)piperidin-1-yl)-3-oxopropyl)benzonitrile;
1-(4-((5-(1H-pyrazol-4-yl)-1H-pyrrolo[2,3-c]pyridin-1-yl)methyl)piperidin-1-yl)-3-(3-fluorophenyl)propan-1-one;
3-(3-(4-((5-(1H-pyrazol-4-yl)-1H-pyrrolo[2,3-c]pyridin-1-yl)methyl)piperidin-1-yl)-3-oxopropyl)benzonitrile;
(4-((5-(1H-pyrazol-4-yl)-1H-pyrrolo[2,3-c]pyridin-1-yl)methyl)-2,2-dimethylpyrrolidin-1-yl)(2-fluorophenyl)methanone;
((4-((5-(1H-pyrazol-4-yl)-1H-pyrrolo[2,3-c]pyridin-1-yl)methyl)-2,2-dimethylpyrrolidin-1-yl)(4-fluorophenyl)methanone;
(4-((5-(1H-pyrazol-4-yl)-1H-pyrrolo[3,2-b]pyridin-1-yl)methyl)-2,2-dimethylpyrrolidin-1-yl)(2,3-difluorophenyl)methanone;
((4-((5-(1H-pyrazol-4-yl)-1H-pyrrolo[2,3-c]pyridin-1-yl)methyl)-2,2-dimethylpyrrolidin-1-yl)(3-methoxyphenyl)methanone;
(4-((5-(1H-pyrazol-4-yl)-1H-pyrrolo[2,3-c]pyridin-1-yl)methyl)-2,2-dimethylpyrrolidin-1-yl)(3-fluorophenyl)methanone;
(4-((5-(1H-pyrazol-4-yl)-1H-pyrrolo[2,3-c]pyridin-1-yl)methyl)-2,2-dimethylpyrrolidin-1-yl)(phenyl)methanone;
1-(4-((5-(1H-pyrazol-4-yl)-1H-pyrrolo[2,3-c]pyridin-1-yl)methyl)-2,2-dimethylpyrrolidin-1-yl)-3-(2-fluorophenyl)propan-1-one;
11-(4-((5-(1H-pyrazol-4-yl)-1H-pyrrolo[2,3-c]pyridin-1-yl)methyl)-2,2-dimethylpyrrolidin-1-yl)-3-phenylpropan-1-one;
rac.-cis-(4-((5-(1H-pyrazol-4-yl)-1H-pyrrolo[2,3-c]pyridin-1-yl)methyl)-2-methylpyrrolidin-1-yl)(2-fluorophenyl)methanone;
rac.-cis-(4-((5-(1H-pyrazol-4-yl)-1H-pyrrolo[2,3-c]pyridin-1-yl)methyl)-2-methylpyrrolidin-1-yl)(4-fluorophenyl)methanone;
rac.-cis-(4-((5-(1H-pyrazol-4-yl)-1H-pyrrolo[2,3-c]pyridin-1-yl)methyl)-2-methylpyrrolidin-1-yl)(2,3-difluorophenyl)methanone;
rac.-cis-(-4-((5-(1H-pyrazol-4-yl)-1H-pyrrolo[2,3-c]pyridin-1-yl)methyl)-2-methylpyrrolidin-1-yl)(phenyl)methanone;
rac.-trans-(4-((5-(1H-pyrazol-4-yl)-1H-pyrrolo[2,3-c]pyridin-1-yl)methyl)-2-methylpyrrolidin-1-yl)(2-fluorophenyl)methanone;
rac.-trans-(4-((5-(1H-pyrazol-4-yl)-1H-pyrrolo[2,3-c]pyridin-1-yl)methyl)-2-methylpyrrolidin-1-yl)(4-fluorophenyl)methanone;
rac.-trans-(4-((5-(1H-pyrazol-4-yl)-1H-pyrrolo[2,3-c]pyridin-1-yl)methyl)-2-methylpyrrolidin-1-yl)(2,3-difluorophenyl)methanone;
rac.-cis-(-4-((5-(1H-pyrazol-4-yl)-1H-pyrrolo[2,3-c]pyridin-1-yl)methyl)-2-methylpyrrolidin-1-yl)(phenyl)methanone;
rac.-cis-1-(4-((5-(1H-pyrazol-4-yl)-1H-pyrrolo[3,2-b]pyridin-1-yl)methyl)-2-methylpyrrolidin-1-yl)-3-phenylpropan-1-one;
rac.-cis-1-(4-((5-(1H-pyrazol-4-yl)-1H-pyrrolo[2,3-c]pyridin-1-yl)methyl)-2-methylpyrrolidin-1-yl)-3-(4-fluorophenyl)propan-1-one;
rac.-trans-1-(4-((5-(1H-pyrazol-4-yl)-1H-pyrrolo[3,2-b]pyridin-1-yl)methyl)-2-methylpyrrolidin-1-yl)-3-phenylpropan-1-one;
rac.-trans-1-(4-((5-(1H-pyrazol-4-yl)-1H-pyrrolo[2,3-c]pyridin-1-yl)methyl)-2-methylpyrrolidin-1-yl)-3-(4-fluorophenyl)propan-1-one;
(4-((5-(1H-pyrazol-4-yl)-1H-pyrrolo[2,3-c]pyridin-1-yl)methyl)-2-methylpyrrolidin-1-yl)(phenyl)methanone;
1-(4-((5-(1H-pyrazol-4-yl)-1H-pyrrolo[2,3-c]pyridin-1-yl)methyl)-2-methylpyrrolidin-1-yl)-2-phenylethan-1-one;
1-(4-((5-(1H-pyrazol-4-yl)-1H-pyrrolo[2,3-c]pyridin-1-yl)methyl)-2-methylpyrrolidin-1-yl)-3-phenylpropan-1-one;
(4-((5-(1H-pyrazol-4-yl)-1H-pyrrolo[2,3-c]pyridin-1-yl)methyl)azepan-1-yl)(phenyl)methanone;
1-(4-((5-(1H-pyrazol-4-yl)-1H-pyrrolo[2,3-c]pyridin-1-yl)methyl)azepan-1-yl)-2-phenylethan-1-one;
rac.-(2-((5-(1H-pyrazol-4-yl)-1H-pyrrolo[2,3-c]pyridin-1-yl)methyl)-7-azabicyclo[2.2.1]heptan-7-yl)(phenyl)methanone;
rac.-1-(-2-((5-(1H-pyrazol-4-yl)-1H-pyrrolo[2,3-c]pyridin-1-yl)methyl)-7-azabicyclo[2.2.1]heptan-7-yl)-3-methylbutan-1-one;
rac.-1-(2-((5-(1H-pyrazol-4-yl)-1H-pyrrolo[2,3-c]pyridin-1-yl)methyl)-7-azabicyclo[2.2.1]heptan-7-yl)-3-phenylpropan-1-one;
1-(4-((5-(5-methyl-1H-pyrazol-4-yl)-1H-pyrrolo[2,3-c]pyridin-1-yl)methyl)piperidin-1-yl)-3-phenylpropan-1-one;
(4-((5-(1H-pyrazol-4-yl)-1H-pyrrolo[2,3-b]pyridin-1-yl)methyl)piperidin-1-yl)(2-fluorophenyl)methanone;
(4-((5-(1H-pyrazol-4-yl)-1H-pyrrolo[2,3-b]pyridin-1-yl)methyl)piperidin-1-yl)(4-fluorophenyl)methanone;
(4-((5-(1H-pyrazol-4-yl)-1H-pyrrolo[2,3-b]pyridin-1-yl)methyl)piperidin-1-yl)(2,3-difluorophenyl)methanone;
(4-((5-(1H-pyrazol-4-yl)-1H-pyrrolo[2,3-b]pyridin-1-yl)methyl)piperidin-1-yl)(2,4-difluorophenyl)methanone;
(4-((5-(1H-pyrazol-4-yl)-1H-pyrrolo[2,3-b]pyridin-1-yl)methyl)piperidin-1-yl)(p-tolyl)methanone;
4-(4-((5-(1H-pyrazol-4-yl)-1H-pyrrolo[2,3-b]pyridin-1-yl)methyl)piperidine-1-carbonyl)benzonitrile;
(4-((5-(1H-pyrazol-4-yl)-1H-pyrrolo[2,3-b]pyridin-1-yl)methyl)piperidin-1-yl)(3-fluorophenyl)methanone;
3-(4-((5-(1H-pyrazol-4-yl)-1H-pyrrolo[2,3-b]pyridin-1-yl)methyl)piperidine-1-carbonyl)benzonitrile;
(4-((5-(1H-pyrazol-4-yl)-1H-pyrrolo[2,3-b]pyridin-1-yl)methyl)piperidin-1-yl)(2,6-difluorophenyl)methanone;
1-(4-((5-(1H-pyrazol-4-yl)-1H-pyrrolo[2,3-b]pyridin-1-yl)methyl)piperidin-1-yl)-3-(2-fluorophenyl)propan-1-one;
11-(4-((5-(1H-pyrazol-4-yl)-1H-pyrrolo[2,3-b]pyridin-1-yl)methyl)piperidin-1-yl)-3-(4-fluorophenyl)propan-1-one;
1-(4-((5-(1H-pyrazol-4-yl)-1H-pyrrolo[2,3-b]pyridin-1-yl)methyl)piperidin-1-yl)-3-(2,3-difluorophenyl)propan-1-one;
1-(4-((5-(1H-pyrazol-4-yl)-1H-pyrrolo[2,3-b]pyridin-1-yl)methyl)piperidin-1-yl)-3-(2,4-difluorophenyl)propan-1-one;
1-(4-((5-(1H-pyrazol-4-yl)-1H-pyrrolo[2,3-b]pyridin-1-yl)methyl)piperidin-1-yl)-3-(p-tolyl)propan-1-one;
4-(3-(4-((5-(1H-pyrazol-4-yl)-1H-pyrrolo[2,3-b]pyridin-1-yl)methyl)piperidin-1-yl)-3-oxopropyl)benzonitrile;
1-(4-((5-(1H-pyrazol-4-yl)-1H-pyrrolo[2,3-b]pyridin-1-yl)methyl)piperidin-1-yl)-3-(3-fluorophenyl)propan-1-one;
3-(3-(4-((5-(1H-pyrazol-4-yl)-1H-pyrrolo[2,3-b]pyridin-1-yl)methyl)piperidin-1-yl)-3-oxopropyl)benzonitrile;
(4-((5-(1H-pyrazol-4-yl)-1H-pyrrolo[2,3-b]pyridin-1-yl)methyl)-2,2-dimethylpyrrolidin-1-yl)(2-fluorophenyl)methanone;
((4-((5-(1H-pyrazol-4-yl)-1H-pyrrolo[2,3-b]pyridin-1-yl)methyl)-2,2-dimethylpyrrolidin-1-yl)(4-fluorophenyl)methanone;
(4-((5-(1H-pyrazol-4-yl)-1H-pyrrolo[3,2-b]pyridin-1-yl)methyl)-2,2-dimethylpyrrolidin-1-yl)(2,3-difluorophenyl)methanone;
((4-((5-(1H-pyrazol-4-yl)-1H-pyrrolo[2,3-b]pyridin-1-yl)methyl)-2,2-dimethylpyrrolidin-1-yl)(3-methoxyphenyl)methanone;
(4-((5-(1H-pyrazol-4-yl)-1H-pyrrolo[2,3-b]pyridin-1-yl)methyl)-2,2-dimethylpyrrolidin-1-yl)(3-fluorophenyl)methanone;
(4-((5-(1H-pyrazol-4-yl)-1H-pyrrolo[2,3-b]pyridin-1-yl)methyl)-2,2-dimethylpyrrolidin-1-yl)(phenyl)methanone;
1-(4-((5-(1H-pyrazol-4-yl)-1H-pyrrolo[2,3-b]pyridin-1-yl)methyl)-2,2-dimethylpyrrolidin-1-yl)-3-(2-fluorophenyl)propan-1-one;
11-(4-((5-(1H-pyrazol-4-yl)-1H-pyrrolo[2,3-b]pyridin-1-yl)methyl)-2,2-dimethylpyrrolidin-1-yl)-3-phenylpropan-1-one;
rac.-cis-(4-((5-(1H-pyrazol-4-yl)-1H-pyrrolo[2,3-b]pyridin-1-yl)methyl)-2-methylpyrrolidin-1-yl)(2-fluorophenyl)methanone;
rac.-cis-(4-((5-(1H-pyrazol-4-yl)-1H-pyrrolo[2,3-b]pyridin-1-yl)methyl)-2-methylpyrrolidin-1-yl)(4-fluorophenyl)methanone;
rac.-cis-(4-((5-(1H-pyrazol-4-yl)-1H-pyrrolo[2,3-b]pyridin-1-yl)methyl)-2-methylpyrrolidin-1-yl)(2,3-difluorophenyl)methanone;
rac.-cis-(-4-((5-(1H-pyrazol-4-yl)-1H-pyrrolo[2,3-b]pyridin-1-yl)methyl)-2-methylpyrrolidin-1-yl)(phenyl)methanone;
rac.-trans-(4-((5-(1H-pyrazol-4-yl)-1H-pyrrolo[2,3-b]pyridin-1-yl)methyl)-2-methylpyrrolidin-1-yl)(2-fluorophenyl)methanone;
rac.-trans-(4-((5-(1H-pyrazol-4-yl)-1H-pyrrolo[2,3-b]pyridin-1-yl)methyl)-2-methylpyrrolidin-1-yl)(4-fluorophenyl)methanone;
rac.-trans-(4-((5-(1H-pyrazol-4-yl)-1H-pyrrolo[2,3-b]pyridin-1-yl)methyl)-2-methylpyrrolidin-1-yl)(2,3-difluorophenyl)methanone;
rac.-cis-(-4-((5-(1H-pyrazol-4-yl)-1H-pyrrolo[2,3-b]pyridin-1-yl)methyl)-2-methylpyrrolidin-1-yl)(phenyl)methanone;
rac.-cis-1-(4-((5-(1H-pyrazol-4-yl)-1H-pyrrolo[3,2-b]pyridin-1-yl)methyl)-2-methylpyrrolidin-1-yl)-3-phenylpropan-1-one;
rac.-cis-1-(4-((5-(1H-pyrazol-4-yl)-1H-pyrrolo[2,3-b]pyridin-1-yl)methyl)-2-methylpyrrolidin-1-yl)-3-(4-fluorophenyl)propan-1-one;
rac.-trans-1-(4-((5-(1H-pyrazol-4-yl)-1H-pyrrolo[3,2-b]pyridin-1-yl)methyl)-2-methylpyrrolidin-1-yl)-3-phenylpropan-1-one;
rac.-trans-1-(4-((5-(1H-pyrazol-4-yl)-1H-pyrrolo[2,3-b]pyridin-1-yl)methyl)-2-methylpyrrolidin-1-yl)-3-(4-fluorophenyl)propan-1-one;
(4-((5-(1H-pyrazol-4-yl)-1H-pyrrolo[2,3-b]pyridin-1-yl)methyl)-2-methylpyrrolidin-1-yl)(phenyl)methanone;
1-(4-((5-(1H-pyrazol-4-yl)-1H-pyrrolo[2,3-b]pyridin-1-yl)methyl)-2-methylpyrrolidin-1-yl)-2-phenylethan-1-one;
1-(4-((5-(1H-pyrazol-4-yl)-1H-pyrrolo[2,3-b]pyridin-1-yl)methyl)-2-methylpyrrolidin-1-yl)-3-phenylpropan-1-one;
(4-((5-(1H-pyrazol-4-yl)-1H-pyrrolo[2,3-b]pyridin-1-yl)methyl)azepan-1-yl)(phenyl)methanone;
1-(4-((5-(1H-pyrazol-4-yl)-1H-pyrrolo[2,3-b]pyridin-1-yl)methyl)azepan-1-yl)-2-phenylethan-1-one;
rac.-(2-((5-(1H-pyrazol-4-yl)-1H-pyrrolo[2,3-b]pyridin-1-yl)methyl)-7-azabicyclo[2.2.1]heptan-7-yl)(phenyl)methanone;
rac.-1-(-2-((5-(1H-pyrazol-4-yl)-1H-pyrrolo[2,3-b]pyridin-1-yl)methyl)-7-azabicyclo[2.2.1]heptan-7-yl)-3-methylbutan-1-one;
rac.-1-(2-((5-(1H-pyrazol-4-yl)-1H-pyrrolo[2,3-b]pyridin-1-yl)methyl)-7-azabicyclo[2.2.1]heptan-7-yl)-3-phenylpropan-1-one; 1-(4-((5-(5-methyl-1H-pyrazol-4-yl)-1H-pyrrolo[2,3-c]pyridin-1-yl)methyl)piperidin-1-yl)-3-phenylpropan-1-one;
1-(4-((5-(5-methyl-1H-pyrazol-4-yl)-1H-pyrrolo[2,3-b]pyridin-1-yl)methyl)piperidin-1-yl)-3-phenylpropan-1-one;
(2-fluorophenyl)(4-((5-(1-methyl-1H-pyrazol-4-yl)-1H-pyrrolo[2,3-c]pyridin-1-yl)methyl)piperidin-1-yl)methanone;
(2-fluorophenyl)(4-((5-(4-methylpyridin-3-yl)-1H-pyrrolo[2,3-c]pyridin-1-yl)methyl)piperidin-1-yl)methanone;
(4-((5-(1H-pyrazol-4-yl)-1H-pyrrolo[2,3-c]pyridin-1-yl)methyl)-4-fluoropiperidin-1-yl)(2-fluorophenyl)methanone;
(4-((5-(1H-pyrazol-4-yl)-1H-pyrrolo[2,3-c]pyridin-1-yl)methyl)piperidin-1-yl)(pyridin-2-yl)methanone;
(4-((5-(1H-pyrazol-4-yl)-1H-pyrrolo[2,3-c]pyridin-1-yl)methyl)piperidin-1-yl)(pyridazin-3-yl)methanone;
(4-((5-(1H-pyrazol-4-yl)-1H-pyrrolo[2,3-c]pyridin-1-yl)methyl)piperidin-1-yl)(pyrimidin-4-yl)methanone;
(4-((5-(1H-pyrazol-4-yl)-1H-pyrrolo[2,3-c]pyridin-1-yl)methyl)piperidin-1-yl)(cyclohexyl)methanone;
1-(4-((5-(1H-pyrazol-4-yl)-1H-pyrrolo[2,3-c]pyridin-1-yl)methyl)piperidin-1-yl)-2-cyclohexylethan-1-one;
(4-((5-(1H-pyrazol-4-yl)-1H-pyrrolo[2,3-c]pyridin-1-yl)methyl)piperidin-1-yl)(4-(difluoromethoxy)phenyl)methanone;
1-(4-((5-(1H-pyrazol-4-yl)-1H-pyrrolo[2,3-c]pyridin-1-yl)methyl)piperidin-1-yl)-4,4,4-trifluoro-3-hydroxy-3-(trifluoromethyl)butan-1-one;
(4-((5-(1H-pyrazol-4-yl)-1H-pyrrolo[2,3-c]pyridin-1-yl)methyl)piperidin-1-yl)(2-chlorophenyl)methanone;
1-(4-((5-(1H-pyrazol-4-yl)-1H-pyrrolo[2,3-c]pyridin-1-yl)methyl)piperidin-1-yl)-2-phenylethan-1-one; or
(4-((5-(1H-pyrazol-4-yl)-1H-pyrrolo[2,3-c]pyridin-1-yl)methyl)piperidin-1-yl)(2-(trifluoromethyl)phenyl)methanone.

51. A pharmaceutical composition, comprising a therapeutically effective amount of a compound according to paragraph 1 and a pharmaceutically acceptable carrier.

52. A pharmaceutical composition, comprising a therapeutically effective amount of a compound according to paragraph 32 and a pharmaceutically acceptable carrier.

53. A method of treating a Retinoic Acid Receptor-Related Orphan Receptor mediated disease or disorder, comprising the step of administering a therapeutically effective amount of a compound according to paragraph 1 to a patient in need thereof.

54. A method of treating a Retinoic Acid Receptor-Related Orphan Receptor mediated disease or disorder, comprising the step of administering a therapeutically effective amount of a compound according to paragraph 32 to a patient in need thereof.

55. A method of treating a Retinoic Acid Receptor-Related Orphan Receptor mediated disease or disorder, comprising the step of administering a therapeutically effective amount of a compound according to paragraph 1 to a patient in need thereof, wherein said disease or disorder is an autoimmune, inflammatory, metabolic or oncologic disease or disorder.

56. A method of treating a Retinoic Acid Receptor-Related Orphan Receptor mediated disease or disorder, comprising the step of administering a therapeutically effective amount of a compound according to paragraph 1 to a patient in need thereof, wherein said disease or disorder is rheumatoid arthritis, psoriasis, psoriatic arthritis, polymyalgia rheumatica, multiple sclerosis, lupus, uveitis, inflammatory bowel disease, ankylosing spondylitis, vasculitis, atherosclerosis, macular degeneration, diabetes, obesity, cancer, asthma or chronic obstructive pulmonary disease.

It is to be understood that the invention is not limited to the particular embodiments of the invention described above, as variations of the particular embodiments may be made and still fall within the scope of the appended claims.

1. A compound of formula (I): 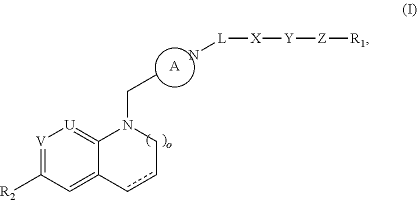
wherein:
A is a mono- or bicyclic 5- to 8-membered heterocyclic ring having one ring carbon replaced by N as shown, said ring optionally mono- or bi-substituted on one or more ring carbons independently with a C₁-C₆ alkyl group, or halogen;
L is —C(O)— or —S(O)₂;
X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
One of U and V is N and the other is CH;
R₁ is —C₁-C₆ alkyl, optionally mono- or bi-substituted with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O); [symbol]
is a single or double bond;
o is 0 or 1;
n is 0 or 1;
p is 0, 1 or 2; and
q is 0 or 1,
or a pharmaceutically acceptable salt thereof.

2. The compound according to claim 1, wherein A is unsubstituted piperidinyl, pyrrolidinyl, azepanyl or azabicyclo[2.2.1]heptanyl.

3. The compound according to claim 1, wherein A is piperidinyl, pyrrolidinyl, azabicyclo[2.2.1]heptanyl or azepanyl mono- or bi-substituted independently with C₁-C₆ alkyl or halogen.

4. The compound according to claim 1, wherein A is piperidinyl, pyrrolidinyl, azepanyl or azabicyclo[2.2.1]heptanyl mono-substituted with methyl or fluorine.

5. The compound according to claim 1, wherein A is piperidinyl, pyrrolidinyl or azepanyl bi-substituted with methyl or fluorine.

6. The compound according to claim 1, wherein X is —CH₂—, —O— or —NH—.

7. The compound according to claim 1, wherein Y is —O—.

8. The compound according to claim 1, wherein R₁ is —C₁-C₆ alkyl, optionally substituted independently with perfluoroalkyl or —OH.

9. The compound according to claim 1, wherein R₁ is methyl, ethyl, propyl or t-butyl.

10. The compound according to claim 1, wherein R₁ is unsubstituted phenyl.

11. The compound according to claim 1, wherein R₁ is phenyl substituted with halogen, alkoxy or C₁-C₆ alkyl.

12. The compound according to claim 1, wherein R₁ is chlorophenyl, dichlorophenyl, fluorophenyl or difluorophenyl.

13. The compound according to claim 1, wherein R₁ is methoxy-phenyl.

14. The compound according to claim 1, wherein R₁ is methyl-phenyl.

15. The compound according to claim 1, wherein R₁ is cycloalkyl.

16. The compound according to claim 1, wherein R₁ is cyclopentyl or cyclohexyl.

17. The compound according to claim 1, wherein R₁ is an unsubstituted 5- or 6-membered heteroaryl group having one or more ring carbons replaced by N.

18. The compound according to claim 1, wherein R₁ is a 5- or 6-membered heteroaryl group, having one or more ring carbons replaced by N, optionally substituted with a C₁-C₆ alkyl group.

19. The compound according to claim 1, wherein R₂ is an unsubstituted 5- to 7-membered heteroaryl group having one, two or three ring carbons replaced by N.

20. The compound according to claim 1, wherein R₂ is a 5- to 7-membered heteroaryl group having one, two or three ring carbons replaced by N, mono- or bi-substituted independently with C₁-C₆ alkyl, —CN or (═O).

21. The compound according to claim 1, wherein R₂ is unsubstituted pyrazolyl, pyridinyl or triazolyl.

22. The compound according to claim 1, wherein R₂ is pyrazolyl, pyridinyl or triazolyl, mono- or bi-substituted independently with methyl, nitrile or CF₃.

23. The compound according to claim 1, wherein R₂ is unsubstituted pyrazolyl.

24. The compound according to claim 1, wherein R₂ is linked via a carbon atom.

25. The compound according to claim 1, having a formula (Ia): 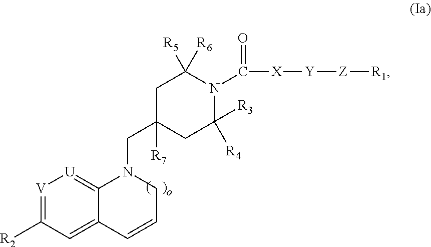
wherein:
X is —(CH₂)ₙ—, —O— or —NH—.
Y is —(CH₂)ₚ—, —O—, —S— or —SO₂—, with the proviso that X and Y are not both a heteroatom;
Z is —(CH₂)_{q}—;
One of U and V is N and the other is CH;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl or
a 5- or 6-membered heteroaryl group having one or more ring carbons independently replaced by N, O or S, said heteroaryl optionally mono- or bi-substituted independently with halogen, alkoxy, C₁-C₆ alkyl, —CN, or perfluorinated C₁-C₆ alkyl;
R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O);
R₃, R₄, R₅ and R₆ are, independently of each other, H or —C₁-C₆ alkyl;
R₇ is H or F; [symbol]
is a single or double bond;
o is 0 or 1;
n is 0 or 1;
p is 0, 1 or 2; and
q is 0 or 1,
or a pharmaceutically acceptable salt thereof.

26. The compound according to claim 1, having a formula (Ib): 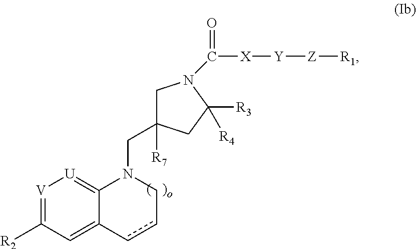
wherein:
X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
One of U and V is N and the other is CH;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O);
R₃ and R₄ are, independently of each other, H or —C₁-C₆ alkyl;
R₇ is H or F; [symbol]
is a single or double bond;
o is 0 or 1;
n is 0 or 1;
p is 0, 1 or 2; and
q is 0 or 1,
or a pharmaceutically acceptable salt thereof.

27. The compound according to claim 1, having a formula (Ic): 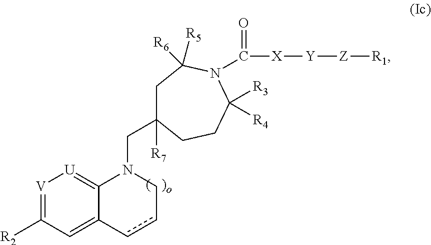
wherein:
X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
One of U and V is N and the other is CH;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O);
R₃, R₄ R₅, and R₆ are, independently of each other, H or —C₁-C₆ alkyl;
R₇ is H or F; [symbol]
is a single or double bond;
o is 0 or 1;
n is 0 or 1;
p is 0, 1 or 2; and
q is 0 or 1,
or a pharmaceutically acceptable salt thereof.

28. The compound according to claim 1, having a formula (Id): 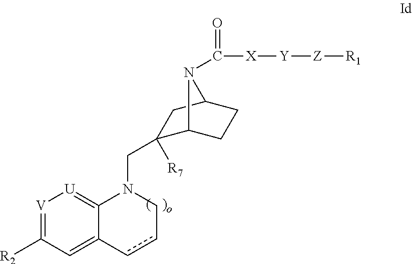
wherein:
X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
One of U and V is N and the other is CH;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O);
R₇ is H or F; [symbol]
is a single or double bond;
o is 0 or 1;
n is 0 or 1;
p is 0, 1 or 2; and
q is 0 or 1,
or a pharmaceutically acceptable salt thereof.

29. The compound according to claim 1, having a formula (Ie): 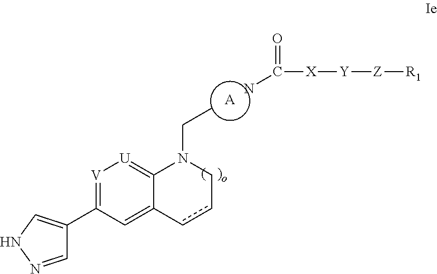
wherein:
A is a monocyclic or bicyclic 5- to 8-membered heterocyclic ring having one ring carbon replaced by N as shown, said ring optionally mono- or bi-substituted on one or more ring carbons independently with a C₁-C₆ alkyl group, or halogen;
X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
One of U and V is N and the other is CH;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O); [symbol]
is a single or double bond;
o is 0 or 1;
n is 0 or 1;
p is 0, 1 or 2; and
q is 0 or 1,
or a pharmaceutically acceptable salt thereof.

30. The compound according to claim 1, having a formula (If): 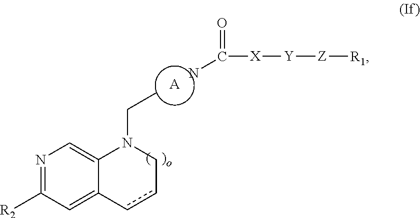
wherein:
A is a monocyclic or bicyclic 5- to 8-membered heterocyclic ring having one ring carbon replaced by N as shown, said ring optionally mono- or bi-substituted on one or more ring carbons independently with a C₁-C₆ alkyl group, or halogen;
X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O);
o is 0 or 1;
n is 0 or 1;
p is 0, 1 or 2; and
q is 0 or 1,
or a pharmaceutically acceptable salt thereof.

31. The compound according to claim 1, having a formula (Ig): 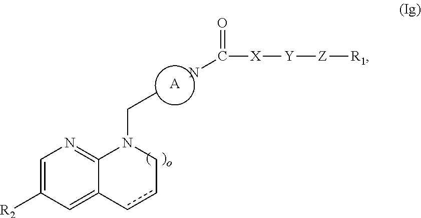
wherein:
A is a monocyclic or bicyclic 5- to 8-membered heterocyclic ring having one ring carbon replaced by N as shown, said ring optionally mono- or bi-substituted on one or more ring carbons independently with a C₁-C₆ alkyl group, or halogen;
X is —(CH₂)ₙ—, —O—, —NH— or absent;
Y is —(CH₂)ₚ—, —O—, —S—, —SO₂— or absent, with the proviso that X and Y are not both a heteroatom;
Z is —(CH₂)_{q}— or absent;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, or perfluorinated C₁-C₆ alkyl,
cycloalkyl, optionally mono- or bi-substituted independently with halogen, alkoxy, C₁-C₆ alkyl, —CN, or perfluorinated C₁-C₆ alkyl,
heterocycle, optionally mono- or bi-substituted independently with halogen, alkoxy, C₁-C₆ alkyl, —CN, or perfluorinated C₁-C₆ alkyl, or
a 5- or 6-membered heteroaryl group having one or more ring carbons independently replaced by N, O or S, said heteroaryl optionally mono- or bi-substituted independently with halogen, alkoxy, C₁-C₆ alkyl, —CN or perfluorinated C₁-C₆ alkyl;
R₂ is a 5- to 7-membered heteroaryl group having one, two or three ring carbons independently replaced by N, O or S, said heteroaryl optionally mono- or bi-substituted independently with C₁-C₆ alkyl, CF₃, —CN or (═O); [symbol]
is a single or double bond;
o is 0 or 1;
n is 0 or 1;
p is 0, 1 or 2; and
q is 0 or 1,
or a pharmaceutically acceptable salt thereof.

32. The compound according to claim 1, having a formula I(h): 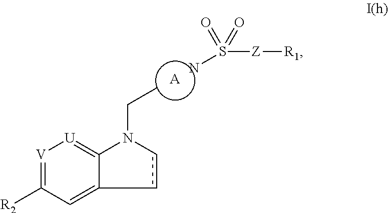
wherein:
A is a monocyclic or bicyclic 5- to 8-membered heterocyclic ring having one ring carbon replaced by N as shown, said ring optionally mono- or bi-substituted on one or more ring carbons independently with a C₁-C₆ alkyl group, or halogen;
One of U and V is N and the other is CH;
Z is —(CH₂)_{q}— or abent;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, alkylsulfonyloxy, alkylsulfonyl, halo-C₁-C₆ alkyl or C₁-C₅ cycloalkyl,
cycloalkyl, optionally mono- or bi-substituted independently with halogen, alkoxy, C₁-C₆ alkyl, —CN, alkylsulfonyloxy, alkylsulfonyl, halo-C₁-C₆ alkyl or C₁-C₅ cycloalkyl,
heterocycle, optionally mono- or bi-substituted independently with halogen, alkoxy, C₁-C₆ alkyl, —CN, alkylsulfonyloxy, alkylsulfonyl, halo-C₁-C₆ alkyl or C₁-C₅ cycloalkyl or
a 5- or 6-membered heteroaryl group having one or more ring carbons independently replaced by N, O or S, said heteroaryl optionally substituted with halogen, alkoxy, C₁-C₆ alkyl, —CN, or perfluorinated C₁-C₆ alkyl;
R₂ is a 5- to 7-membered heteroaryl group having one, two or three ring carbons independently replaced by N, O or S, said heteroaryl optionally mono- or bi-substituted independently with C₁-C₆ alkyl, —CN or (═O); [symbol]
is a single or double bond; and
q is 0, 1 or 2.
or a pharmaceutically acceptable salt thereof.

33. The compound of claim 32, wherein U is N and V is CH.

34. The compound of claim 32, wherein U is CH and V is N.

35. The compound of claim 32, wherein A is unsubstituted piperidinyl, pyrrolidinyl, bicyclo[2,2,1]azepinyl or azepanyl.

36. The compound of claim 32, wherein A is piperidinyl, pyrrolidinyl, azepanyl or bicyclo[2,2,1]azepinyl mono- or bi-substituted independently with C₁-C₆ alkyl or halogen.

37. The compound of claim 32, wherein A is piperidinyl, pyrrolidinyl, azepanyl or bicyclo[2,2,1]azepinyl mono-substituted with methyl or fluorine.

38. The compound of claim 32, wherein A is piperidinyl, pyrrolidinyl, azepanyl or bicyclo[2,2,1]azepinyl bi-substituted independently with methyl or fluorine.

39. The compound of claim 32, wherein R₁ is —C₁-C₆ alkyl optionally substituted with perfluoroalkyl.

40. The compound of claim 32, wherein R₁ is methyl, ethyl, propyl or t-butyl.

41. The compound of claim 32, wherein R₁ is unsubstituted phenyl.

42. The compound of claim 32, wherein R₁ is phenyl substituted with halogen, alkylsulfonyl, alkoxy, —CN, alkyl, or C₁-C₆ alkyl.

43. The compound of claim 32, wherein R₁ is cycloalkyl.

44. The compound of claim 32, wherein R₁ is an unsubstituted 5- or 6-membered heteroaryl group having one or more ring carbons replaced by N.

45. The compound of claim 32, wherein R₂ is an unsubstituted 5- to 7-membered heteroaryl group having one, two or three ring carbons replaced by N.

46. The compound of claim 32, wherein R₂ is unsubstituted pyrazolyl or triazolyl.

47. The compound of claim 32, wherein R₂ is unsubstituted pyrazolyl.

48. The compound of claim 32, wherein R₂ is linked via a carbon atom.

49. The compound according to claim 1, having a formula I(i): 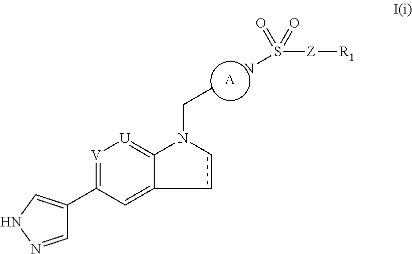
wherein:
A is a monocyclic or bicyclic 5- to 8-membered heterocyclic ring having one ring carbon replaced by N as shown, said ring optionally mono- or bi-substituted on one or more ring carbons independently with a C₁-C₆ alkyl group, or halogen;
One of U and V is N and the other is CH;
Z is —(CH₂)_{q}— or absent;
R₁ is —C₁-C₆ alkyl, optionally mono- or bi-substituted independently with —OH, halogen or —CN,
phenyl, optionally mono- or bi-substituted independently with halogen, alkoxy, C₁-C₆ alkyl, —CN, alkylsulfonyloxy, alkylsulfonyl, halo-C₁-C₆ alkyl or C₁-C₅ cycloalkyl,
cycloalkyl, optionally mono- or bi-substituted independently with halogen, alkoxy, C₁-C₆ alkyl, —CN, alkylsulfonyloxy, alkylsulfonyl, halo-C₁-C₆ alkyl or C₁-C₅ cycloalkyl,
heterocycle, optionally mono- or bi-substituted independently with halogen, alkoxy, C₁-C₆ alkyl, —CN, alkylsulfonyloxy, alkylsulfonyl, halo-C₁-C₆ alkyl or C₁-C₅ cycloalkyl or
a 5- or 6-membered heteroaryl group having one or more ring carbons independently replaced by N, O or S, said heteroaryl optionally mono- or bi-substituted independently with halogen, alkoxy, C₁-C₆ alkyl, —CN, or perfluorinated C₁-C₆ alkyl; [symbol]
is a single or double bond; and
q is 0, 1 or 2.
or a pharmaceutically acceptable salt thereof.

50. The compound according to claim 1, wherein said compound is:
(4-((5-(1H-pyrazol-4-yl)-1H-pyrrolo[2,3-c]pyridin-1-yl)methyl)piperidin-1-yl)(phenyl)methanone;
1-(4-((5-(1H-pyrazol-4-yl)-1H-pyrrolo[2,3-c]pyridin-1-yl)methyl)piperidin-1-yl)-3-phenylpropan-1-one;
(4-((5-(1H-pyrazol-4-yl)-1H-pyrrolo[2,3-c]pyridin-1-yl)methyl)piperidin-1-yl)(phenyl)methanone;
(4-((5-(1H-pyrazol-4-yl)-1H-pyrrolo[2,3-c]pyridin-1-yl)methyl)piperidin-1-yl)(2-fluorophenyl)methanone;
(4-((5-(1H-pyrazol-4-yl)-1H-pyrrolo[2,3-c]pyridin-1-yl)methyl)piperidin-1-yl)(2-fluorophenyl)methanone;
(4-((5-(1H-pyrazol-4-yl)-1H-pyrrolo[2,3-c]pyridin-1-yl)methyl)piperidin-1-yl)(2-fluorophenyl)methanone;
1-((1-(phenethyl sulfonyl)piperidin-4-yl)methyl)-5-(1H-pyrazol-4-yl)-1H-pyrrolo[2,3-c]pyri dine;
1-((1-(isobutylsulfonyl)piperidin-4-yl)methyl)-5-(1H-pyrazol-4-yl)-1H-pyrrolo[2,3-c]pyridine;
1-((1-(phenylsulfonyl)piperidin-4-yl)methyl)-5-(1H-pyrazol-4-yl)-1H-pyrrolo[2,3-c]pyridine;
1-((1-((2-fluorophenyl)sulfonyl)piperidin-4-yl)methyl)-5-(1H-pyrazol-4-yl)-1H-pyrrolo[2,3-c]pyridine;
(4-((5-(1H-pyrazol-4-yl)-1H-pyrrolo[2,3-b]pyridin-1-yl)methyl)piperidin-1-yl)(phenyl)methanone;
1-(4-((5-(1H-pyrazol-4-yl)-1H-pyrrolo[2,3-b]pyridin-1-yl)methyl)piperidin-1-yl)-3-phenylpropan-1-one;
1-(4-((5-(1H-pyrazol-4-yl)-1H-pyrrolo[2,3-b]pyridin-1-yl)methyl)piperidin-1-yl)-3-methylbutan-1-one;
(4-((5-(1H-pyrazol-4-yl)-1H-pyrrolo[2,3-b]pyridin-1-yl)methyl)piperidin-1-yl)(2-fluorophenyl)methanone;
1-((1-(phenethylsulfonyl)piperidin-4-yl)methyl)-5-(1H-pyrazol-4-yl)-1H-pyrrolo[2,3-b]pyridine;
1-((1-(isobutylsulfonyl)piperidin-4-yl)methyl)-5-(1H-pyrazol-4-yl)-1H-pyrrolo[2,3-b]pyridine;
1-((1-(phenylsulfonyl)piperidin-4-yl)methyl)-5-(1H-pyrazol-4-yl)-1H-pyrrolo[2,3-b]pyridine;
1-((1-((2-fluorophenyl)sulfonyl)piperidin-4-yl)methyl)-5-(1H-pyrazol-4-yl)-1H-pyrrolo[2,3-b]pyridine;
(4-((5-(1H-pyrazol-4-yl)-1H-pyrrolo[2,3-c]pyridin-1-yl)methyl)piperidin-1-yl)(2-(trifluoromethyl)phenyl)methanone;
(4-((5-(1H-pyrazol-4-yl)-1H-pyrrolo[2,3-c]pyridin-1-yl)methyl)piperidin-1-yl)(4-fluorophenyl)methanone;
(4-((5-(1H-pyrazol-4-yl)-1H-pyrrolo[2,3-c]pyridin-1-yl)methyl)piperidin-1-yl)(2,3-difluorophenyl)methanone;
(4-((5-(1H-pyrazol-4-yl)-1H-pyrrolo[2,3-c]pyridin-1-yl)methyl)piperidin-1-yl)(2,4-difluorophenyl)methanone;
(4-((5-(1H-pyrazol-4-yl)-1H-pyrrolo[2,3-c]pyridin-1-yl)methyl)piperidin-1-yl)(p-tolyl)methanone;
4-(4-((5-(1H-pyrazol-4-yl)-1H-pyrrolo[2,3-c]pyridin-1-yl)methyl)piperidine-1-carbonyl)benzonitrile;
(4-((5-(1H-pyrazol-4-yl)-1H-pyrrolo[2,3-c]pyridin-1-yl)methyl)piperidin-1-yl)(3-fluorophenyl)methanone;
3-(4-((5-(1H-pyrazol-4-yl)-1H-pyrrolo[2,3-c]pyridin-1-yl)methyl)piperidine-1-carbonyl)benzonitrile;
(4-((5-(1H-pyrazol-4-yl)-1H-pyrrolo[2,3-c]pyridin-1-yl)methyl)piperidin-1-yl)(2,6-difluorophenyl)methanone;
1-(4-((5-(1H-pyrazol-4-yl)-1H-pyrrolo[2,3-c]pyridin-1-yl)methyl)piperidin-1-yl)-3-(2-fluorophenyl)propan-1-one;
11-(4-((5-(1H-pyrazol-4-yl)-1H-pyrrolo[2,3-c]pyridin-1-yl)methyl)piperidin-1-yl)-3-(4-fluorophenyl)propan-1-one;
1-(4-((5-(1H-pyrazol-4-yl)-1H-pyrrolo[2,3-c]pyridin-1-yl)methyl)piperidin-1-yl)-3-(2,3-difluorophenyl)propan-1-one;
1-(4-((5-(1H-pyrazol-4-yl)-1H-pyrrolo[2,3-c]pyridin-1-yl)methyl)piperidin-1-yl)-3-(2,4-difluorophenyl)propan-1-one;
1-(4-((5-(1H-pyrazol-4-yl)-1H-pyrrolo[2,3-c]pyridin-1-yl)methyl)piperidin-1-yl)-3-(p-tolyl)propan-1-one;
4-(3-(4-((5-(1H-pyrazol-4-yl)-1H-pyrrolo[2,3-c]pyridin-1-yl)methyl)piperidin-1-yl)-3-oxopropyl)benzonitrile;
1-(4-((5-(1H-pyrazol-4-yl)-1H-pyrrolo[2,3-c]pyridin-1-yl)methyl)piperidin-1-yl)-3-(3-fluorophenyl)propan-1-one;
3-(3-(4-((5-(1H-pyrazol-4-yl)-1H-pyrrolo[2,3-c]pyridin-1-yl)methyl)piperidin-1-yl)-3-oxopropyl)benzonitrile;
(4-((5-(1H-pyrazol-4-yl)-1H-pyrrolo[2,3-c]pyridin-1-yl)methyl)-2,2-dimethylpyrrolidin-1-yl)(2-fluorophenyl)methanone;
((4-((5-(1H-pyrazol-4-yl)-1H-pyrrolo[2,3-c]pyridin-1-yl)methyl)-2,2-dimethylpyrrolidin-1-yl)(4-fluorophenyl)methanone;
(4-((5-(1H-pyrazol-4-yl)-1H-pyrrolo[3,2-b]pyridin-1-yl)methyl)-2,2-dimethylpyrrolidin-1-yl)(2,3-difluorophenyl)methanone;
((4-((5-(1H-pyrazol-4-yl)-1H-pyrrolo[2,3-c]pyridin-1-yl)methyl)-2,2-dimethylpyrrolidin-1-yl)(3-methoxyphenyl)methanone;
(4-((5-(1H-pyrazol-4-yl)-1H-pyrrolo[2,3-c]pyridin-1-yl)methyl)-2,2-dimethylpyrrolidin-1-yl)(3-fluorophenyl)methanone;
(4-((5-(1H-pyrazol-4-yl)-1H-pyrrolo[2,3-c]pyridin-1-yl)methyl)-2,2-dimethylpyrrolidin-1-yl)(phenyl)methanone;
1-(4-((5-(1H-pyrazol-4-yl)-1H-pyrrolo[2,3-c]pyridin-1-yl)methyl)-2,2-dimethylpyrrolidin-1-yl)-3-(2-fluorophenyl)propan-1-one;
11-(4-((5-(1H-pyrazol-4-yl)-1H-pyrrolo[2,3-c]pyridin-1-yl)methyl)-2,2-dimethylpyrrolidin-1-yl)-3-phenylpropan-1-one;
rac.-cis-(4-((5-(1H-pyrazol-4-yl)-1H-pyrrolo[2,3-c]pyridin-1-yl)methyl)-2-methylpyrrolidin-1-yl)(2-fluorophenyl)methanone;
rac.-cis-(4-((5-(1H-pyrazol-4-yl)-1H-pyrrolo[2,3-c]pyridin-1-yl)methyl)-2-methylpyrrolidin-1-yl)(4-fluorophenyl)methanone;
rac.-cis-(4-((5-(1H-pyrazol-4-yl)-1H-pyrrolo[2,3-c]pyridin-1-yl)methyl)-2-methylpyrrolidin-1-yl)(2,3-difluorophenyl)methanone;
rac.-cis-(-4-((5-(1H-pyrazol-4-yl)-1H-pyrrolo[2,3-c]pyridin-1-yl)methyl)-2-methylpyrrolidin-1-yl)(phenyl)methanone;
rac.-trans-(4-((5-(1H-pyrazol-4-yl)-1H-pyrrolo[2,3-c]pyridin-1-yl)methyl)-2-methylpyrrolidin-1-yl)(2-fluorophenyl)methanone;
rac.-trans-(4-((5-(1H-pyrazol-4-yl)-1H-pyrrolo[2,3-c]pyridin-1-yl)methyl)-2-methylpyrrolidin-1-yl)(4-fluorophenyl)methanone;
rac.-trans-(4-((5-(1H-pyrazol-4-yl)-1H-pyrrolo[2,3-c]pyridin-1-yl)methyl)-2-methylpyrrolidin-1-yl)(2,3-difluorophenyl)methanone;
rac.-cis-(-4-((5-(1H-pyrazol-4-yl)-1H-pyrrolo[2,3-c]pyridin-1-yl)methyl)-2-methylpyrrolidin-1-yl)(phenyl)methanone;
rac.-cis-1-(4-((5-(1H-pyrazol-4-yl)-1H-pyrrolo[3,2-b]pyridin-1-yl)methyl)-2-methylpyrrolidin-1-yl)-3-phenylpropan-1-one;
rac.-cis-1-(4-((5-(1H-pyrazol-4-yl)-1H-pyrrolo[2,3-c]pyridin-1-yl)methyl)-2-methylpyrrolidin-1-yl)-3-(4-fluorophenyl)propan-1-one;
rac.-trans-1-(4-((5-(1H-pyrazol-4-yl)-1H-pyrrolo[3,2-b]pyridin-1-yl)methyl)-2-methylpyrrolidin-1-yl)-3-phenylpropan-1-one;
rac.-trans-1-(4-((5-(1H-pyrazol-4-yl)-1H-pyrrolo[2,3-c]pyridin-1-yl)methyl)-2-methylpyrrolidin-1-yl)-3-(4-fluorophenyl)propan-1-one;
(4-((5-(1H-pyrazol-4-yl)-1H-pyrrolo[2,3-c]pyridin-1-yl)methyl)-2-methylpyrrolidin-1-yl)(phenyl)methanone;
1-(4-((5-(1H-pyrazol-4-yl)-1H-pyrrolo[2,3-c]pyridin-1-yl)methyl)-2-methylpyrrolidin-1-yl)-2-phenylethan-1-one;
1-(4-((5-(1H-pyrazol-4-yl)-1H-pyrrolo[2,3-c]pyridin-1-yl)methyl)-2-methylpyrrolidin-1-yl)-3-phenylpropan-1-one;
(4-((5-(1H-pyrazol-4-yl)-1H-pyrrolo[2,3-c]pyridin-1-yl)methyl)azepan-1-yl)(phenyl)methanone;
1-(4-((5-(1H-pyrazol-4-yl)-1H-pyrrolo[2,3-c]pyridin-1-yl)methyl)azepan-1-yl)-2-phenylethan-1-one;
rac.-(2-((5-(1H-pyrazol-4-yl)-1H-pyrrolo[2,3-c]pyridin-1-yl)methyl)-7-azabicyclo[2.2.1]heptan-7-yl)(phenyl)methanone;
rac.-1-(-2-((5-(1H-pyrazol-4-yl)-1H-pyrrolo[2,3-c]pyridin-1-yl)methyl)-7-azabicyclo[2.2.1]heptan-7-yl)-3-methylbutan-1-one;
rac.-1-(2-((5-(1H-pyrazol-4-yl)-1H-pyrrolo[2,3-c]pyridin-1-yl)methyl)-7-azabicyclo[2.2.1]heptan-7-yl)-3-phenylpropan-1-one;
1-(4-((5-(5-methyl-1H-pyrazol-4-yl)-1H-pyrrolo[2,3-c]pyridin-1-yl)methyl)piperidin-1-yl)-3-phenylpropan-1-one;
(4-((5-(1H-pyrazol-4-yl)-1H-pyrrolo[2,3-b]pyridin-1-yl)methyl)piperidin-1-yl)(2-fluorophenyl)methanone;
(4-((5-(1H-pyrazol-4-yl)-1H-pyrrolo[2,3-b]pyridin-1-yl)methyl)piperidin-1-yl)(4-fluorophenyl)methanone;
(4-((5-(1H-pyrazol-4-yl)-1H-pyrrolo[2,3-b]pyridin-1-yl)methyl)piperidin-1-yl)(2,3-difluorophenyl)methanone;
(4-((5-(1H-pyrazol-4-yl)-1H-pyrrolo[2,3-b]pyridin-1-yl)methyl)piperidin-1-yl)(2,4-difluorophenyl)methanone;
(4-((5-(1H-pyrazol-4-yl)-1H-pyrrolo[2,3-b]pyridin-1-yl)methyl)piperidin-1-yl)(p-tolyl)methanone;
4-(4-((5-(1H-pyrazol-4-yl)-1H-pyrrolo[2,3-b]pyridin-1-yl)methyl)piperidine-1-carbonyl)benzonitrile;
(4-((5-(1H-pyrazol-4-yl)-1H-pyrrolo[2,3-b]pyridin-1-yl)methyl)piperidin-1-yl)(3-fluorophenyl)methanone;
3-(4-((5-(1H-pyrazol-4-yl)-1H-pyrrolo[2,3-b]pyridin-1-yl)methyl)piperidine-1-carbonyl)benzonitrile;
(4-((5-(1H-pyrazol-4-yl)-1H-pyrrolo[2,3-b]pyridin-1-yl)methyl)piperidin-1-yl)(2,6-difluorophenyl)methanone;
1-(4-((5-(1H-pyrazol-4-yl)-1H-pyrrolo[2,3-b]pyridin-1-yl)methyl)piperidin-1-yl)-3-(2-fluorophenyl)propan-1-one;
11-(4-((5-(1H-pyrazol-4-yl)-1H-pyrrolo[2,3-b]pyridin-1-yl)methyl)piperidin-1-yl)-3-(4-fluorophenyl)propan-1-one;
1-(4-((5-(1H-pyrazol-4-yl)-1H-pyrrolo[2,3-b]pyridin-1-yl)methyl)piperidin-1-yl)-3-(2,3-difluorophenyl)propan-1-one;
1-(4-((5-(1H-pyrazol-4-yl)-1H-pyrrolo[2,3-b]pyridin-1-yl)methyl)piperidin-1-yl)-3-(2,4-difluorophenyl)propan-1-one;
1-(4-((5-(1H-pyrazol-4-yl)-1H-pyrrolo[2,3-b]pyridin-1-yl)methyl)piperidin-1-yl)-3-(p-tolyl)propan-1-one;
4-(3-(4-((5-(1H-pyrazol-4-yl)-1H-pyrrolo[2,3-b]pyridin-1-yl)methyl)piperidin-1-yl)-3-oxopropyl)benzonitrile;
1-(4-((5-(1H-pyrazol-4-yl)-1H-pyrrolo[2,3-b]pyridin-1-yl)methyl)piperidin-1-yl)-3-(3-fluorophenyl)propan-1-one;
3-(3-(4-((5-(1H-pyrazol-4-yl)-1H-pyrrolo[2,3-b]pyridin-1-yl)methyl)piperidin-1-yl)-3-oxopropyl)benzonitrile;
(4-((5-(1H-pyrazol-4-yl)-1H-pyrrolo[2,3-b]pyridin-1-yl)methyl)-2,2-dimethylpyrrolidin-1-yl)(2-fluorophenyl)methanone;
((4-((5-(1H-pyrazol-4-yl)-1H-pyrrolo[2,3-b]pyridin-1-yl)methyl)-2,2-dimethylpyrrolidin-1-yl)(4-fluorophenyl)methanone;
(4-((5-(1H-pyrazol-4-yl)-1H-pyrrolo[3,2-b]pyridin-1-yl)methyl)-2,2-dimethylpyrrolidin-1-yl)(2,3-difluorophenyl)methanone;
((4-((5-(1H-pyrazol-4-yl)-1H-pyrrolo[2,3-b]pyridin-1-yl)methyl)-2,2-dimethylpyrrolidin-1-yl)(3-methoxyphenyl)methanone;
(4-((5-(1H-pyrazol-4-yl)-1H-pyrrolo[2,3-b]pyridin-1-yl)methyl)-2,2-dimethylpyrrolidin-1-yl)(3-fluorophenyl)methanone;
(4-((5-(1H-pyrazol-4-yl)-1H-pyrrolo[2,3-b]pyridin-1-yl)methyl)-2,2-dimethylpyrrolidin-1-yl)(phenyl)methanone;
1-(4-((5-(1H-pyrazol-4-yl)-1H-pyrrolo[2,3-b]pyridin-1-yl)methyl)-2,2-dimethylpyrrolidin-1-yl)-3-(2-fluorophenyl)propan-1-one;
11-(4-((5-(1H-pyrazol-4-yl)-1H-pyrrolo[2,3-b]pyridin-1-yl)methyl)-2,2-dimethylpyrrolidin-1-yl)-3-phenylpropan-1-one;
rac.-cis-(4-((5-(1H-pyrazol-4-yl)-1H-pyrrolo[2,3-b]pyridin-1-yl)methyl)-2-methylpyrrolidin-1-yl)(2-fluorophenyl)methanone;
rac.-cis-(4-((5-(1H-pyrazol-4-yl)-1H-pyrrolo[2,3-b]pyridin-1-yl)methyl)-2-methylpyrrolidin-1-yl)(4-fluorophenyl)methanone;
rac.-cis-(4-((5-(1H-pyrazol-4-yl)-1H-pyrrolo[2,3-b]pyridin-1-yl)methyl)-2-methylpyrrolidin-1-yl)(2,3-difluorophenyl)methanone;
rac.-cis-(-4-((5-(1H-pyrazol-4-yl)-1H-pyrrolo[2,3-b]pyridin-1-yl)methyl)-2-methylpyrrolidin-1-yl)(phenyl)methanone;
rac.-trans-(4-((5-(1H-pyrazol-4-yl)-1H-pyrrolo[2,3-b]pyridin-1-yl)methyl)-2-methylpyrrolidin-1-yl)(2-fluorophenyl)methanone;
rac.-trans-(4-((5-(1H-pyrazol-4-yl)-1H-pyrrolo[2,3-b]pyridin-1-yl)methyl)-2-methylpyrrolidin-1-yl)(4-fluorophenyl)methanone;
rac.-trans-(4-((5-(1H-pyrazol-4-yl)-1H-pyrrolo[2,3-b]pyridin-1-yl)methyl)-2-methylpyrrolidin-1-yl)(2,3-difluorophenyl)methanone;
rac.-cis-(-4-((5-(1H-pyrazol-4-yl)-1H-pyrrolo[2,3-b]pyridin-1-yl)methyl)-2-methylpyrrolidin-1-yl)(phenyl)methanone;
rac.-cis-1-(4-((5-(1H-pyrazol-4-yl)-1H-pyrrolo[3,2-b]pyridin-1-yl)methyl)-2-methylpyrrolidin-1-yl)-3-phenylpropan-1-one;
rac.-cis-1-(4-((5-(1H-pyrazol-4-yl)-1H-pyrrolo[2,3-b]pyridin-1-yl)methyl)-2-methylpyrrolidin-1-yl)-3-(4-fluorophenyl)propan-1-one;
rac.-trans-1-(4-((5-(1H-pyrazol-4-yl)-1H-pyrrolo[3,2-b]pyridin-1-yl)methyl)-2-methylpyrrolidin-1-yl)-3-phenylpropan-1-one;
rac.-trans-1-(4-((5-(1H-pyrazol-4-yl)-1H-pyrrolo[2,3-b]pyridin-1-yl)methyl)-2-methylpyrrolidin-1-yl)-3-(4-fluorophenyl)propan-1-one;
(4-((5-(1H-pyrazol-4-yl)-1H-pyrrolo[2,3-b]pyridin-1-yl)methyl)-2-methylpyrrolidin-1-yl)(phenyl)methanone;
1-(4-((5-(1H-pyrazol-4-yl)-1H-pyrrolo[2,3-b]pyridin-1-yl)methyl)-2-methylpyrrolidin-1-yl)-2-phenylethan-1-one;
1-(4-((5-(1H-pyrazol-4-yl)-1H-pyrrolo[2,3-b]pyridin-1-yl)methyl)-2-methylpyrrolidin-1-yl)-3-phenylpropan-1-one;
(4-((5-(1H-pyrazol-4-yl)-1H-pyrrolo[2,3-b]pyridin-1-yl)methyl)azepan-1-yl)(phenyl)methanone;
1-(4-((5-(1H-pyrazol-4-yl)-1H-pyrrolo[2,3-b]pyridin-1-yl)methyl)azepan-1-yl)-2-phenylethan-1-one;
rac.-(2-((5-(1H-pyrazol-4-yl)-1H-pyrrolo[2,3-b]pyridin-1-yl)methyl)-7-azabicyclo[2.2.1]heptan-7-yl)(phenyl)methanone;
rac.-1-(-2-((5-(1H-pyrazol-4-yl)-1H-pyrrolo[2,3-b]pyridin-1-yl)methyl)-7-azabicyclo[2.2.1]heptan-7-yl)-3-methylbutan-1-one;
rac.-1-(2-((5-(1H-pyrazol-4-yl)-1H-pyrrolo[2,3-b]pyridin-1-yl)methyl)-7-azabicyclo[2.2.1]heptan-7-yl)-3-phenylpropan-1-one; 1-(4-((5-(5-methyl-1H-pyrazol-4-yl)-1H-pyrrolo[2,3-c]pyridin-1-yl)methyl)piperidin-1-yl)-3-phenylpropan-1-one;
1-(4-((5-(5-methyl-1H-pyrazol-4-yl)-1H-pyrrolo[2,3-b]pyridin-1-yl)methyl)piperidin-1-yl)-3-phenylpropan-1-one;
(2-fluorophenyl)(4-((5-(1-methyl-1H-pyrazol-4-yl)-1H-pyrrolo[2,3-c]pyridin-1-yl)methyl)piperidin-1-yl)methanone;
(2-fluorophenyl)(4-((5-(4-methylpyridin-3-yl)-1H-pyrrolo[2,3-c]pyridin-1-yl)methyl)piperidin-1-yl)methanone;
(4-((5-(1H-pyrazol-4-yl)-1H-pyrrolo[2,3-c]pyridin-1-yl)methyl)-4-fluoropiperidin-1-yl)(2-fluorophenyl)methanone;
(4-((5-(1H-pyrazol-4-yl)-1H-pyrrolo[2,3-c]pyridin-1-yl)methyl)piperidin-1-yl)(pyridin-2-yl)methanone;
(4-((5-(1H-pyrazol-4-yl)-1H-pyrrolo[2,3-c]pyridin-1-yl)methyl)piperidin-1-yl)(pyridazin-3-yl)methanone;
(4-((5-(1H-pyrazol-4-yl)-1H-pyrrolo[2,3-c]pyridin-1-yl)methyl)piperidin-1-yl)(pyrimidin-4-yl)methanone;
(4-((5-(1H-pyrazol-4-yl)-1H-pyrrolo[2,3-c]pyridin-1-yl)methyl)piperidin-1-yl)(cyclohexyl)methanone;
1-(4-((5-(1H-pyrazol-4-yl)-1H-pyrrolo[2,3-c]pyridin-1-yl)methyl)piperidin-1-yl)-2-cyclohexylethan-1-one;
(4-((5-(1H-pyrazol-4-yl)-1H-pyrrolo[2,3-c]pyridin-1-yl)methyl)piperidin-1-yl)(4-(difluoromethoxy)phenyl)methanone;
1-(4-((5-(1H-pyrazol-4-yl)-1H-pyrrolo[2,3-c]pyridin-1-yl)methyl)piperidin-1-yl)-4,4,4-trifluoro-3-hydroxy-3-(trifluoromethyl)butan-1-one;
(4-((5-(1H-pyrazol-4-yl)-1H-pyrrolo[2,3-c]pyridin-1-yl)methyl)piperidin-1-yl)(2-chlorophenyl)methanone;
1-(4-((5-(1H-pyrazol-4-yl)-1H-pyrrolo[2,3-c]pyridin-1-yl)methyl)piperidin-1-yl)-2-phenylethan-1-one; or
(4-((5-(1H-pyrazol-4-yl)-1H-pyrrolo[2,3-c]pyridin-1-yl)methyl)piperidin-1-yl)(2-(trifluoromethyl)phenyl)methanone.

51. A pharmaceutical composition, comprising a therapeutically effective amount of a compound according to claim 1 and a pharmaceutically acceptable carrier.

52. A pharmaceutical composition, comprising a therapeutically effective amount of a compound according to claim 32 and a pharmaceutically acceptable carrier.

53. A method of treating a Retinoic Acid Receptor-Related Orphan Receptor mediated disease or disorder, comprising the step of administering a therapeutically effective amount of a compound according to claim 1 to a patient in need thereof.

54. A method of treating a Retinoic Acid Receptor-Related Orphan Receptor mediated disease or disorder, comprising the step of administering a therapeutically effective amount of a compound according to claim 32 to a patient in need thereof.

55. A method of treating a Retinoic Acid Receptor-Related Orphan Receptor mediated disease or disorder, comprising the step of administering a therapeutically effective amount of a compound according to claim 1 to a patient in need thereof, wherein said disease or disorder is an autoimmune, inflammatory, metabolic or oncologic disease or disorder.

56. A method of treating a Retinoic Acid Receptor-Related Orphan Receptor mediated disease or disorder, comprising the step of administering a therapeutically effective amount of a compound according to claim 1 to a patient in need thereof, wherein said disease or disorder is rheumatoid arthritis, psoriasis, psoriatic arthritis, polymyalgia rheumatica, multiple sclerosis, lupus, uveitis, inflammatory bowel disease, ankylosing spondylitis, vasculitis, atherosclerosis, macular degeneration, diabetes, obesity, cancer, asthma or chronic obstructive pulmonary disease.
